(19) United States
(12) Patent Application Publication
(10) Pub. No.: US 2015/0197503 A1
(43) Pub. Date: Jul. 16, 2015

(54) PROCESS FOR THE SYNTHESIS OF SUBSTITUTED UREA COMPOUNDS

(71) Applicant: Bial-Portela & Cª, S.A., S. Mamede Do Coronado (PT)

(72) Inventors: Domenico Russo, S. Mamede Do Coronado (PT); Jorge Bruno Reis Wahnon, S. Mamede Do Coronado (PT); William Maton, S. Mamede Do Coronado (PT); Tibor Eszenyi, Tiszalok (HU)

(21) Appl. No.: 14/417,354

(22) PCT Filed: Jul. 26, 2013
(86) PCT No.: PCT/PT2013/000050

§ 371 Date: Jan. 26, 2015

Related U.S. Application Data
(60) Provisional application No. 61/676,554, filed on Jul. 27, 2012.

(51) Int. Cl. C07D 401/04; C07D 213/50; C07D 213/53

(52) U.S. Cl. CPC C07D 401/04; C07D 213/53; C07D 213/50

(57) ABSTRACT

A process for preparing a substituted urea compound of Formula II or Formula I, or a pharmaceutically acceptable salt or ester thereof, Formula II, Formula I the process comprising the reaction of an intermediate of Formula II′ or Formula 1′, Formula II′, Formula I′ with a carbamoyl halide of the formula: R1R2NC(=0)Hal, in a solvent consisting essentially of pyridine, wherein Hal represents Cl, F, I or Br, and wherein ring A, and R1, R2, R5, V, W, X, Y and Z are as defined herein.

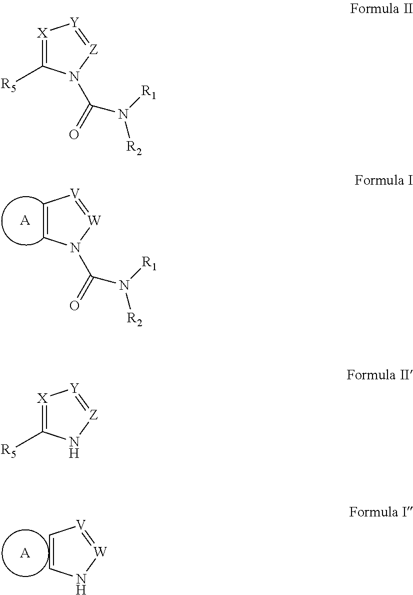

The present invention relates to processes for the synthesis of substituted urea compounds and of intermediates useful in the production of such compounds. In particular, though not exclusively, it relates to processes for synthesising certain active pharmaceutical ingredients having a heteroaryl N-carboxamide core, and novel intermediates used in such processes.

Molecules containing urea functional groups are of interest in medicinal chemistry. A common method for their preparation is to convert a first amine component to an isocyanate or activated carbamate, followed by reaction with a second amine component. However, this approach is not available when neither of the amine components is a primary amine. In particular, secondary amines cannot be converted to isocyanates, and secondary carbamates are known to suffer from low reactivity in the required nucleophilic substitution reaction with the second amine component (see Lee et al. (2004) Tetrahedron 60, 3439). Complex or harsh approaches have thus been used in these circumstances, e.g. the aluminium amide approach described by Lee et al. (above).

A number of molecules having fatty acid amide hydrolase (FAAH) inhibitory activity and containing urea groups are disclosed in WO 2010074588, the entire contents of which, and in particular the details of the compounds claimed therein, are hereby incorporated herein. For example, a subgroup of the compounds disclosed in this document contain an imidazole-1-carboxamide motif. These compounds are generally prepared using an approach comprising carbamoylation of 1H-imidazole derivatives with carbamoyl chlorides. For illustrative purposes, 3-(1-(cyclohexyl(methyl)carbamoyl)-1H-imidazol-4-yl)pyridine-1-oxide, hereinafter sometimes referred to as compound A, is prepared by reaction of the imidazolylpyridine hydrochloride with potassium 2-methylpropan-2-olate in a mixed solvent of tetrahydrofuran (THF) and dimethylformamide (DMF), followed by addition of a catalytic amount of pyridine and N,N-dimethylpyridine-4-amine, this step being followed by addition of cyclohexyl(methyl)carbamic chloride. This mixture is kept at elevated temperature overnight, following which a non-oxidised intermediate can be extracted in low yield. This intermediate is then oxidised to give compound A. A similar approach to urea formation using cyclohexyl(methyl)carbamic chloride is described in Koga et al. (1998) Bioorg. Med. Chem. Lett. 8, 1471. The solvent used for urea formation in this instance is DMF.

The main limitation of the above procedure disclosed in WO 2010074588 is the very low overall yield. This problem is addressed in WO2012015324, wherein the ureas of WO2010074588 are synthesised using an alternative approach based on the reaction of a phenylcarbamate derivative of an N-containing heteroaryl group with a primary or secondary amine. The yield using the phenylcarbamate approach is reported to be much improved, and WO2012015324 discourages the use of the carbamoyl chloride approach.

Therefore, there exists a need to provide an efficient approach for the formation of substituted ureas, particularly (but not exclusively) those containing an imidazole-1-carboxamide core.

According to one aspect of the present invention, there is provided a process for preparing a substituted urea compound of Formula II or Formula I, or a pharmaceutically acceptable salt or ester thereof,

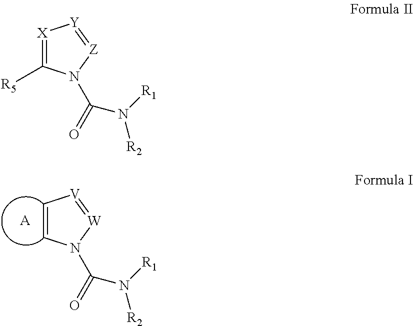

the process comprising the reaction of an intermediate of Formula II′ or Formula I′,

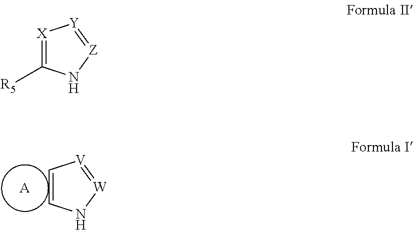

with a carbamoyl halide of the formula: R1R2NC(═O)Hal, in a solvent consisting essentially of pyridine,
wherein Hal represents Cl, F, I or Br,
wherein R1 and R2 can each be independently selected from H, C₁₋₂₀ alkyl, C₁₋₆ alkoxy, aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, R1a, halogen, OH, OR1a, OCOR1a, SH, SR1a, SCOR1a, NH₂, NHR1a, NHSO₂NH₂, NHSO₂R1a, NR1aCOR1b, NHCOR1a, NR1aR1b, COR1a, CSR1a, CN, COOH, COOR1a, CONH₂, CONHOH, CONHR1a, CONHOR1a, SO₂R1a, SO₃H, SO₂NH₂, CONR1aR1b, SO₂NR1aR1b, wherein R1a and R1b are independently selected from C₁₋₆ alkyl, substituted C₁₋₆ alkyl, aryl, heteroaryl, C₃₋₈ cycloalkyl and heterocyclyl, or R1a and R1b, together with the heteroatom to which they are joined, can form heterocyclyl,
wherein, when R1 or R2 is C₁₋₂₀ alkyl, alkoxy, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, or is a group containing one or more of these moieties, each of these moieties may optionally be substituted with one or more groups selected from R1c, halogen, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₁₀ alkyl, OH, OR1c, OCOR1c, SH, SR1c, SCOR1c, NH₂, NO₂, NHR1c, NHSO₂NH₂, NHSO₂R1c, NR1cCOR1d, NHC(NH)NH₂, NHCOR1c, NR1cR1d, COR1c, CSR1c, CN, COOH, COOR1c, CONH₂, CONHOH, CONHR1c, CONHOR1c, C(NOH)NH₂, CONR1cR1d, SO₂R1c, SO₃H, SO₂NH₂, SO₂NR1cR1d, wherein R1c and R1d are independently selected from C₁₋₆ alkyl, substituted C₁₋₆ alkyl, aryl, heteroaryl, C₃₋₈ cycloalkyl and heterocyclyl, or R1c and R1d, together with the heteroatom to which they are joined, can form heterocyclyl,
wherein, when the substituent of R1 or R2 is C₁₋₁₀ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₆ alkyl, C₃₋₈ cycloalkyl or is a group containing one or more of these moieties, each of these moieties may optionally be substituted with one or more groups selected from R1e, halogen, C₁₋₁₀ alkyl, OH, OR1e, OCOR1e, SH, SR1e, SCOR1e, NH₂, NO₂, NHR1e, NHSO₂NH₂, NHSO₂R1e, NR1eCOR1f, NHC(NH)NH₂, NHCOR1e, NR1eR1f, COR1e, CSR1e, CN, COOH, COOR1e, CONH₂, CONHOH, CONHR1e, CONHOR1e, C(NOH)NH₂, CONR1eR1f, SO₂R1e, SO₃H, SO₂NH₂, SO₂NR1eR1f, wherein R1e and R1f are independently selected from C₁₋₆ alkyl, substituted C₁₋₆ alkyl, aryl, heteroaryl, C₃₋₈ cycloalkyl and heterocyclyl, or R1e and R1f, together with the heteroatom to which they are joined, can form heterocyclyl,
with the exception that R1 and R2 are not both H;
or
R1 and R2, together with the N to which they are attached, can form a heteroaryl or heterocyclyl group, each of which may optionally be substituted with one or more oxygen atoms or one or more groups selected from aryl, heteroaryl, partially or fully saturated heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, R2a, halogen, OH, OR2a, OCOR2a, SH, SR2a, SCOR2a, NH₂, NO₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, CONR2aR2b, SO₂R2a, SO₃H, SO₂NH₂, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of the heteroaryl or heterocyclyl formed by R1 and R2 together is aryl, heteroaryl, heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, or a group containing one or more of these moieties, each of these moieties may optionally be substituted with one or more groups selected from halogen, hydroxyl, C₁₋₆ alkyl, aryl, heteroaryl, heterocyclyl, C₃₋₈ cycloalkyl, C₁₋₄ alkoxy, aryloxy, heteroaryloxy, heterocyclyloxy, C₃₋₈ cycloalkyloxy, aryl C₁₋₄ alkoxy, heteroaryl C₁₋₆ alkoxy, heterocyclyl C₁₋₄ alkoxy, C₃₋₈ cycloalkyl C₁₋₄ alkoxy, R2c, OR2c, OCOR2c, SH, SR2c, SCOR2c, NH₂, NO₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, CONR2cR2d, SO₂R2c, SO₃H, SO₂NH₂, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
wherein, when the substituent of the substituent of the heteroaryl or heterocyclyl formed by R1 and R2 together is C₁₋₆ alkyl, aryl, heteroaryl, heterocyclyl, C₃₋₈ cycloalkyl, C₁₋₆ alkoxy, aryloxy, heteroaryloxy, heterocyclyloxy, C₃₋₈ cycloalkyloxy, aryl C₁₋₄ alkoxy, heteroaryl C₁₋₄ alkoxy, heterocyclyl C₁₋₄ alkoxy, C₃₋₈ cycloalkyl C₁₋₄ alkoxy, or is a group containing one or more of these moieties, each of these moieties may optionally be substituted with one or more groups selected from C₁₋₄ alkoxy, R2e, halogen, OH, OR2e, OCOR2e, SH, SR2e, SCOR2e, NH₂, NO₂, NHR2e, NHSO₂NH₂, NHSO₂R2e, NR2eCOR2f, NHC(NH)NH₂, NR2eR2f, NHCOR2e, COR2e, CSR2e, CN, COOH, COOR2e, CONH₂, CONHOH, CONHR2e, CONHOR2e, C(NOH)NH₂, CONR2eR2f, SO₂R2e, SO₃H, SO₂NH₂, SO₂NR2eR2f, wherein R2e and R2f are independently selected from C₁₋₆ alkyl, substituted C₁₋₆ alkyl, aryl, heteroaryl, C₃₋₃ cycloalkyl and heterocyclyl, or R2e and R2f, together with the heteroatom to which they are joined, can form heterocyclyl;
Ring A is selected from aryl, heteroaryl and heterocyclyl moieties, each of which may optionally be substituted with one or more groups selected from halogen, C₁₋₆ alkyl, aryl, heteroaryl, heterocyclyl, C₁₋₆ alkoxy, aryloxy, heteroaryloxy, heterocyclyloxy, Ra, C₁₋₁₀ alkyl, OH, ORa, OCORa, SH, SRa, SCORa, NH₂, NO₂, NHRa, NHSO₂NH₂, NHSO₂Ra, NRaCORb, NHCORa, NHC(NH)NH₂, NRaRb, CORa, CSRa, CN, COOH, COORa, CONH₂, CONHRa, CONHOH, CONHORa, C(NOH)NH₂, CONRaRb, SO₂Ra, SO₃H, SO₂NH₂, SO₂NRaRb, wherein Ra and Rb are independently selected from C₁₋₆ alkyl, substituted C₁₋₆ alkyl, aryl, heteroaryl, C₃₋₈ cycloalkyl and heterocyclyl, or Ra and Rb, together with the heteroatom to which they are joined, can form heterocyclyl,
wherein, when Ring A is substituted with C₁₋₆ alkyl, aryl, heteroaryl, heterocyclyl, C₁₋₆ alkoxy, aryloxy, heteroaryloxy, heterocyclyloxy, C₁₋₁₀ alkyl, C₃₋₈ cycloalkyl or is substituted with a group containing one or more of these moieties, each of these moieties may optionally be substituted with one or more groups selected from halogen, Rc, C₁₋₁₀ alkyl, aryl C₁₋₆ alkyl, heteroaryl C₁₋₆ alkyl, heterocyclyl C₁₋₆ alkyl, OH, ORc, OCORc, SH, SRc, SCORc, NH₂, NO₂, NHRc, NHSO₂NH₂, NHSO₂Rc, NRcCORd, NHCORc, NHC(NH)NH₂, NRcRd, CORc, CSRc, CN, COOH, COORc, CONH₂, CONHOH, CONHRc, CONHORc, C(NOH)NH₂, CONRcRd, SO₂Rc, SO₃H, SO₂NH₂, SO₂NRcRd, wherein Rc and Rd are independently selected from C₁₋₆ alkyl, substituted C₁₋₆ alkyl, aryl, heteroaryl, C₃₋₈ cycloalkyl and heterocyclyl, or Rc and Rd, together with the heteroatom to which they are joined, can form heterocyclyl;
V can be N, CH or C—R3, wherein R3 is halogen, C₁₋₁₀ alkyl, aryl, heteroaryl, heterocyclyl, C₃₋₈ cycloalkyl, C₁₋₆ alkoxy, aryloxy, heteroaryloxy, heterocyclyloxy, R3a, OH, OR3a, SH, SR3a, OCOR3a, SCOR3a, NH₂, NO₂, NHR3a, NHSO₂NH₂, NHSO₂R3a, NR3aCOR3b, NHCOR3a, NHC(NH)NH₂, NR3aR3b, COR3a, CSR3a, CN, COOH, COOR3a, CONH₂, CONHOH, CONHR3a, CONHOR3a, C(NOH)NH₂, CONR3aR3b, SO₂R3a, SO₃H, SO₂NH₂, SO₂NR3aR3b, wherein R3a and R3b are independently selected from C₁₋₆ alkyl, substituted C₁₋₆ alkyl, aryl, heteroaryl, C₃₋₈ cycloalkyl and heterocyclyl, or R3a and R3b, together with the heteroatom to which they are joined, can form heterocyclyl,
wherein, when R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R3c, C₁₋₁₀ alkyl, OH, OR3c, OCOR3c, SH, SR3c, SCOR3c, NH₂, NO₂, NHR3c, NHSO₂NH₂, NHSO₂R3c, NR3cCOR3d, NHCOR3c, NHC(NH)NH₂, NR3cR3d, COR3c, CSR3c, CN, COOH, COOR3c, CONH₂, CONHOH, CONHR3c, CONHOR3c, C(NOH)NH₂, CONR3cR3d, SO₂R3c, SO₃H, SO₂NH₂, SO₂NR3cR3d, wherein R3c and R3d are independently selected from C₁₋₆ alkyl, substituted C₁₋₆ alkyl, aryl, heteroaryl, C₃₋₈ cycloalkyl and heterocyclyl, or R3c and R3d, together with the heteroatom to which they are joined, can form heterocyclyl,
wherein, when the substituent of R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, R3e, C₁₋₁₀ alkyl, OH, OR3e, OCOR3e, SH, SR3e, SCOR3e, NH₂, NO₂, NHR3e, NHSO₂NH₂, NHSO₂R3e, NR3eCOR3f, NHCOR3e, NHC(NH)NH₂, NR3eR3f, COR3e, CSR3e, CN, COOH, COOR3e, CONH₂, CONHOH, CONHR3e, CONHOR3e, C(NOH)NH₂, CONR3eR3f, SO₂R3e, SO₃H, SO₂NH₂, SO₂NR3eR3f, wherein R3e and R3f are independently selected from C₁₋₆ alkyl, substituted C₁₋₆ alkyl, aryl, heteroaryl, C₃₋₈ cycloalkyl and heterocyclyl, or R3e and R3f, together with the heteroatom to which they are joined, can form heterocyclyl;
W can be N, CH or C—R4, wherein R4 is halogen, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, R4a, OH, OR4a, SH, SR4a, OCOR4a, SCOR4a, NH₂, NO₂, NHR4a, NHSO₂NH₂, NHSO₂R4a, NR4aCOR4b, NHCOR4a, NHC(NH)NH₂, NR4aR4b, COR4a, CSR4a, CN, COOH, COOR4a, CONH₂, CONHOH, CONHR4a, CONHOR4a, C(NOH)NH₂, CONR4aR4b, SO₂R4a, SO₃H, SO₂NH₂, SO₂NR4aR4b, wherein R4a and R4b are independently selected from C₁₋₆ alkyl, substituted C₁₋₆ alkyl, aryl, heteroaryl, C₃₋₈ cycloalkyl and heterocyclyl, or R4a and R4b, together with the heteroatom to which they are joined, can form heterocyclyl,
wherein, when R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₄ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R4c, C₁₋₁₀ alkyl, OH, OR4c, OCOR4c, SH, SR4c, SCOR4c, NH₂, NO₂, NHR4c, NHSO₂NH₂, NHSO₂R4c, NR4cCOR4d, NHCOR4c, NHC(NH)NH₂, NR4cR4d, COR4c, CSR4c, CN, COOH, COOR4c, CONH₂, CONHOH, CONHR4c, CONHOR4c, C(NOH)NH₂, CONR4cR4d, SO₂R4c, SO₃H, SO₂NH₂, SO₂NR4cR4d, wherein R4c and R4d are independently selected from C₁₋₆ alkyl, substituted C₁₋₆ alkyl, aryl, heteroaryl, C₃₋₈ cycloalkyl and heterocyclyl, or R4c and R4d, together with the heteroatom to which they are joined, can form heterocyclyl, wherein, when the substituent of R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, R4e, C₁₋₁₀ alkyl, OH, OR4e, OCOR4e, SH, SR4e, SCOR4e, NH₂, NO₂, NHR4e, NHSO₂NH₂, NHSO₂R4e, NR4eCOR4f, NHCOR4e, NHC(NH)NH₂, NR4eR4f, COR4e, CSR4e, CN, COOH, COOR4e, CONH₂, CONHOH, CONHR4e, CONHOR4e, C(NOH)NH₂, CONR4eR4f, SO₂R4e, SO₃H, SO₂NH₂, SO₂NR4eR4f, wherein R4e and R4f are independently selected from C₁₋₆ alkyl, substituted C₁₋₆ alkyl, aryl, heteroaryl, C₃₋₈ cycloalkyl and heterocyclyl, or R4e and R4f, together with the heteroatom to which they are joined, can form heterocyclyl;
R5 together with the C to which it is attached, can form a carbonyl group with the double bonds in Formula II rearranged accordingly, or R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NO₂, NHR5a, NHSO₂NH₂, NHSO₂R5a, NR5aCOR5b, NHCOR5a, NHC(NH)NH₂, NR5aR5b, COR5a, CSR5a, CN, COOH, COOR5a, CONH₂, CONHOH, CONHR5a, CONHOR5a, C(NOH)NH₂, CONR5aR5b, SO₂R5a, SO₃H, SO₂NH₂, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl,
wherein, when R5 is C₁₋₆ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R5c, C₁₋₆ alkyl, OH, OR5c, OCOR5c, SH, SR5c, SCOR5c, NH₂, NO₂, NHR5c, NHSO₂NH₂, NHSO₂R5c, NR5cCOR5d, NHCOR5c, NHC(NH)NH₂, NR5cR5d, COR5c, CSR5c, CN, COOH, COOR5c, CONH₂, CONHOH, CONHR5c, CONHOR5c, C(NOH)NH₂, CONR5cR5d, SO₂R5c, SO₃H, SO₂NH₂, SO₂NR5cR5d, wherein R5c and R5d are independently selected from C₁₋₆ alkyl, substituted C₁₋₆ alkyl, aryl, heteroaryl, C₃₋₈ cycloalkyl and heterocyclyl, or R5c and R5d, together with the heteroatom to which they are joined, can form heterocyclyl, wherein, when the substituent of R5 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R5e, C₁₋₆ alkyl, OH, OR5e, OCOR5e, SH, SR5e, SCOR5e, NH₂, NO₂, NHR5e, NHSO₂NH₂, NHSO₂R5e, NR5eCOR5f, NHCOR5e, NHC(NH)NH₂, NR5eR5f, COR5e, CSR5e, CN, COOH, COOR5e, CONH₂, CONHOH, CONHR5e, CONHOR5e, C(NOH)NH₂, CONR5eR5f, SO₂R5e, SO₃H, SO₂NH₂, SO₂NR5eR5f, wherein R5e and R5f are independently selected from C₁₋₆ alkyl, substituted C₁₋₆ alkyl, aryl, heteroaryl, C₃₋₈ cycloalkyl and heterocyclyl, or R5e and R5f, together with the heteroatom to which they are joined, can form heterocyclyl;

X can be O (with the double bonds in Formula II rearranged accordingly), N, CH or C—R6, wherein R6 is selected from C₁₋₆ alkyl, aryl, heteroaryl, heterocyclyl, C₁₋₆ alkoxy, aryloxy, heteroaryloxy, heterocyclyloxy, R6a, halogen, OH, OR6a, SH, SR6a, OCOR6a, SCOR6a, NH₂, NO₂, NHR6a, NHSO₂NH₂, NHSO₂R6a, NR6aCOR6b, NHCOR6a, NHC(NH)NH₂, NR6aR6b, COR6a, CSR6a, CN, COOH, COOR6a, CONH₂, CONHOH, CONHR6a, CONHOR6a, C(NOH)NH₂, CONR6aR6b, SO₂R6a, SO₃H, SO₂NH₂, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl,

wherein, when R6 is heteroaryl or heterocyclyl, each of these moieties may optionally be substituted with one or more oxygen atoms, and when R6 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R6c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR6c, OCOR6c, SH, SR6c, SCOR6c, NH₂, NO₂, NHR6c, NHSO₂NH₂, NHC(NH)NH₂, NHSO₂R6c, NR6cCOR6d, NHCOR6c, NR6cR6d, COR6c, CSR6c, CN, COOH, COOR6c, CONH₂, CONHR6c, CONHOR6c, CONHOH, C(NOH)NH₂, CONR6cR6d, SO₂R6c, SO₃H, SO₂NH₂, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R6c and R6d, together with the heteroatom to which they are joined, can form heterocyclyl,
wherein, when the substituent of R6 is heteroaryl or heterocyclyl, each of these moieties may optionally be substituted with one or more oxygen atoms, or when the substituent of R6 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R6e, C₁₋₆ alkyl, C₁₋₄ alkoxy, OH, OR6e, OCOR6e, SH, SR6e, SCOR6e, NH₂, NO₂, NHR6e, NHSO₂NH₂, NHC(NH)NH₂, NHSO₂R6e, NR6eCOR6f, NHCOR6e, NR6eR6f, COR6e, CSR6e, CN, COOH, COOR6e, CONH₂, CONHOH, CONHR6e, CONHOR6e, C(NOH)NH₂, CONR6eR6f, SO₂R6e, SO₃H, SO₂NH₂, SO₂NR6eR6f, wherein R6e and R6f are independently selected from C₁₋₆ alkyl, substituted C₁₋₆ alkyl, aryl, heteroaryl, C₃₋₈ cycloalkyl and heterocyclyl, or R6e and R6f, together with the heteroatom to which they are joined, can form heterocyclyl;
Y can be N, CH or C—R7, wherein R7 is selected from C₁₋₆ alkyl, aryl, heteroaryl, heterocyclyl, C₁₋₆ alkoxy, aryloxy, heteroaryloxy, heterocyclyloxy, R7a, halogen, OH, OR7a, SH, SR7a, OCOR7a, SCOR7a, NH₂, NO₂, NHR7a, NHSO₂NH₂, NHSO₂R7a, NR7aCOR7b, NHCOR7a, NHC(NH)NH₂, NR7aR7b, COR7a, CSR7a, CN, COOH, COOR7a, CONH₂, CONHOH, CONHR7a, CONHOR7a, C(NOH)NH₂, CONR7aR7b, SO₂R7a, SO₃H, SO₂NH₂, SO₂NR7aR7b, wherein R7a and R7b are independently selected from C₁₋₆ alkyl, substituted C₁₋₆ alkyl, aryl, heteroaryl, C₃₋₈ cycloalkyl and heterocyclyl, or R7a and R7b, together with the heteroatom to which they are joined, can form heterocyclyl,
wherein, when R7 is heteroaryl or heterocyclyl, each of these moieties may optionally be substituted with one or more oxygen atoms, and when R7 is C₁₋₆ alkyl, aryl, heteroaryl, heterocyclyl, C₁₋₆ alkoxy, aryloxy, heteroaryloxy, heterocyclyloxy, C₃₋₈ cycloalkyl or is a group containing one or more of these moieties, each of these moieties may optionally be substituted with one or more groups selected from halogen, R7c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, O7c, OCOR7c, SH, SR7c, SCOR7c, NH₂, NO₂, NHR7c, NHSO₂NH₂, NHC(NH)NH₂, NHSO₂R7c, NR7cCOR7d, NHCOR7c, NR7cR7d, COR7c, CSR7c, CN, COOH, COOR7c, CONH₂, CONHR7c, CONHOR7c, CONHOH, C(NOH)NH₂, CONR7cR7d, SO₂R7c, SO₃H, SO₂NH₂, SO₂NR7cR7d, wherein R7c and R7d are independently selected from C₁₋₆ alkyl, substituted C₁₋₆ alkyl, aryl, heteroaryl, C₃₋₈ cycloalkyl and heterocyclyl, or R7c and R7d, together with the heteroatom to which they are joined, can form heterocyclyl,
wherein, when the substituent of R7 is heteroaryl or heterocyclyl, each of these moieties may optionally be substituted with one or more oxygen atoms, or when the substituent of R7 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, aryl, heteroaryl, heterocyclyl, aryl C₁₋₆ alkyl, heteroaryl C₁₋₆ alkyl, heterocyclyl C₁₋₆ alkyl, C₁₋₄ alkoxy, R7e, C₁₋₆ alkyl, OH, OR7e, OCOR7e, SH, SR7e, SCOR7e, NH₂, NO₂, NHR7e, NHSO₂NH₂, NHSO₂R7e, NHC(NH)NH₂, NR7eCOR7f, NHCOR7e, NR7eR7f, COR7e, CSR7e, CN, COOH, COOR7e, CONH₂, CONHOH, CONHR7e, CONHOR7e, C(NOH)NH₂, CONR7eR7f, SO₂R7e, SO₃H, SO₂NH₂, SO₂NR7eR7f, wherein R7e and R7f are independently selected from C₁₋₆ alkyl, substituted C₁₋₆ alkyl, aryl, heteroaryl, C₃₋₈ cycloalkyl and heterocyclyl, or R7e and R7f, together with the heteroatom to which they are joined, can form heterocyclyl;
Z can be N, CH or C—R8, wherein R8 is selected from C₁₋₁₀ alkyl, aryl, heteroaryl, heterocyclyl, C₁₋₆ alkoxy, aryloxy, heteroaryloxy, heterocyclyloxy, R8a, halogen, OH, OR8a, SH, SR8a, OCOR8a, SCOR8a, NH₂, NO₂, NHR8a, NHSO₂NH₂, NHSO₂R8a, NR8aCOR8b, NHCOR8a, NHC(NH)NH₂, NR8aR8b, COR8a, CSR8a, CN, COOH, COOR8a, CONH₂, CONHOH, CONHR8a, CONHOR8a, C(NOH)NH₂, CONR8aR8b, SO₂R8a, SO₃H, SO₂NH₂, SO₂NR8aR8b, wherein R8a and R8b are independently selected from C₁₋₆ alkyl, substituted C₁₋₆ alkyl, aryl, heteroaryl, C₃₋₈ cycloalkyl and heterocyclyl, or R8a and R8b, together with the heteroatom to which they are joined, can form heterocyclyl,
wherein, when R8 is C₁₋₆ alkyl, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R8c, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR8c, OCOR8c, SH, SR8c, SCOR8c, NH₂, NO₂, NHR8c, NHSO₂NH₂, NHSO₂R8c, NR8cCOR8d, NHCOR8c, NHC(NH)NH₂, NR8cR8d, COR8c, CSR8c, CN, COOH, COOR8c, CONH₂, CONHOH, CONHR8c, CONHOR8c, C(NOH)NH₂, CONR8cR8d, SO₂R8c, SO₃H, SO₂NH₂, SO₂NR8cR8d, wherein R8c and R8d are independently selected from C₁₋₆ alkyl, substituted C₁₋₆ alkyl, aryl, heteroaryl, C₃₋₈ cycloalkyl and heterocyclyl, or R8c and R8d, together with the heteroatom to which they are joined, can form heterocyclyl,
wherein, when the substituent of R8 is C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R8e, C₁₋₆ alkyl, OH, OR8e, OCOR8e, SH, SR8e, SCOR8e, NH₂, NO₂, NHR8e, NHSO₂NH₂, NHSO₂R8e, NR8eCOR8f, NHCOR8e, NHC(NH)NH₂, NR8eR8f, COR8e, CSR8e, CN, COOH, COOR8e, CONH₂, CONHOH, CONHR8e, CONHOR8e, C(NOH)NH₂, CONR8eR8f, SO₂R8e, SO₃H, SO₂NH₂, SO₂NR8eR8f, wherein R8e and R8f are independently selected from C₁₋₆ alkyl, substituted C₁₋₆ alkyl, aryl, heteroaryl, cycloalkyl and heterocyclyl, or R8e and R8f, together with the heteroatom to which they are joined, can form heterocyclyl;
wherein, at most, two of the atoms or groups denoted X, Y and Z can be N;
wherein, when W is N, the CONR1R2 group may be joined to W instead, with the double bonds in Formula I rearranged accordingly.

Compared to the processes described in the prior art, the process of the present invention provides a surprisingly beneficial approach to the production of ureas of Formulas II or I. By using pyridine as the solvent for the urea formation reaction, a marked improvement in yield (potentially greater than 90%) is achieved. This compares extremely favourably with a yield of around 7% reported in WO2010074588 (where pyridine is used in catalytic quantities in a DMF/THF solvent), and a yield of around 50% using the phenylcarbamate approach reported in WO2012015324. The process of the invention also leads to marked savings (around 50%) in the cost of input materials compared to the phenylcarbamate approach. The simplicity and beneficial results of the process of the present invention are surprising given the processes described previously.

As mentioned above the processes of the present invention are useful for preparing compounds having FAAH inhibitory activity and containing urea groups, and in particular those compounds disclosed in WO 2010074588, the entire contents of which, and in particular the details of the compounds claimed therein, are hereby incorporated herein by reference. The compounds of WO 2010074588 may be used in a variety of diseases or conditions in which the endogenous endocannabinoid system is implicated. Such conditions include, for example, pain, such as cancer pain.

The solvent used for the reaction of the intermediate of Formula II′ or I′ with the carbamoyl halide consists essentially of pyridine. In the context of the present invention, ‘consists essentially of pyridine’ means that the solvent used for the reaction comprises at least 10% vv pyridine together with other, preferably miscible, solvents. Such other solvents may comprise, for example, dichloromethane or dimethylformamide. Further such solvents include isopropyl alcohol, 2-methyltetrahydrofuran, propionitrile or trifluorotoluene. In certain embodiments, the solvent comprises at least 20%, at least 25%, at least 30%, at least 40%, at least 50%, at least 60%, at least 70%, at least 75%, at least 80%, or at least 90% vv pyridine. Allowing the reaction solvent to contain other solvents means that one or both of the reacting species can be introduced in a solvent other than pyridine, provided that the solvent used for the reaction contains enough pyridine to produce an improvement in yield, as demonstrated by the process described herein. The higher the content of pyridine in the solvent, however, the greater the improvement in yield. The purity of the urea produced is also enhanced by the pyridine solvent

The term ‘C_{x-y} alkyl’ as used herein refers to a linear or branched saturated hydrocarbon group containing from x to y carbon atoms. For example, C₁₋₆ alkyl refers to a linear or branched saturated hydrocarbon group containing from 1 to 6 carbon atoms. Examples of C₁₋₆ alkyl groups include methyl, ethyl, n-propyl, isopropyl, n-butyl, isobutyl, sec-butyl, tert butyl, n-pentyl, isopentyl, neopentyl and hexyl. Preferably, the hydrocarbon group is linear. The group C₁₋₁₀ alkyl is preferably C₁₋₆ alkyl. The tem ‘C_{x-y} alkyl’ is also used to mean a linear or branched saturated hydrocarbon group containing from x to y carbon atoms and in which a terminal methyl group is further substituted, i.e. so as to render a C_{x-y} alkylene group.

The term ‘C_{x-y} alkynyl’ as used herein refers to a linear or branched hydrocarbon group containing from x to y carbon atoms and at least one carbon-carbon triple bond. For example, C₁₋₆ alkynyl refers to a linear or branched hydrocarbon group containing from 1 to 6 carbon atoms. Examples of C₁₋₆ alkynyl groups include, ethynyl, methylbutynyl (e.g. 3-methyl-1-butynyl), 1,3-butadiynyl and 1,3,5-hexatriynyl.

The term ‘aryl’ as used herein refers to a C₆₋₁₂ monocyclic or bicyclic hydrocarbon ring wherein at least one ring is aromatic. Examples of such groups include phenyl, naphthalenyl and tetrahydronaphthalenyl.

The term ‘heteroaryl’ as used herein refers to a 5-6 membered monocyclic aromatic or a fused 8-10 membered bicyclic aromatic ring which monocyclic or bicyclic ring contains 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such bicyclic aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridyl, furopyridyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and imidazopyridyl.

The term ‘heteroaryl substituted with one or more oxygen atoms’ refers to a heteroaryl ring which has one or more oxygen atoms bonded to the ring. It does not mean that the heteroaryl ring contains one or more oxygen atoms as ring atoms, although in some embodiments, this may be the case. Preferably, the one or more oxygen atoms is bonded to a nitrogen heteroatom in the heteroaryl ring. A heteroaryl substituted with an oxygen atom may contain an N-oxide. An example of a heteroaryl substituted with one or more oxygen atoms is 1-oxidopyridyl in which the pyridyl nitrogen is oxidised.

The term ‘heterocyclyl’ refers to a 3-8 (preferably 4-8 and, more preferably, 4-7) membered monocyclic ring or a fused 8-12 membered bicyclic ring which may be saturated or partially unsaturated, which monocyclic or bicyclic ring contains 1 to 4 heteroatoms selected from oxygen, nitrogen, silicon or sulphur. Examples of such monocyclic rings include oxaziridinyl, oxiranyl, dioxiranyl, aziridinyl, pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridyl, tetrahydropyrimidinyl, tetrahydrothiophenyl, tetrahydrothiopyranyl, diazepanyl and azepanyl. Examples of such bicyclic rings include indolinyl, isoindolinyl, benzopyranyl, quinuclidinyl, 2,3,4,5-tetrahydro-1H-3-benzazepine, 4-(benzo[d][1,3]dioxol-5-ylmethyl)piperazin-1-yl, and, tetrahydroisoquinolinyl.

The term ‘heterocyclyl substituted with one or more oxygen atoms’ refers to a heterocyclyl ring which has one or more oxygen atoms bonded to the ring. It does not mean that the heterocyclyl ring contains one or more oxygen atoms as ring atoms, although in some embodiments, this may be the case. Preferably, the one or more oxygen atoms is bonded to a heteroatom, such as nitrogen or sulphur, in the heterocyclyl ring. An example of a heterocyclyl substituted with one or more oxygen atoms is 1,1-dioxido-1,3-thiazolidinyl.

The terms ‘bicyclic ring’ and ‘fused’ in the context of a bicyclic ring refers to two rings which are joined together across a bond between two atoms (e.g. naphthalene), across a sequence of atoms to form a bridge (e.g. quinuclidine) or together at a single atom to form a spiro compound (e.g. 1,4-dioxa-8-aza-spiro[4.5]decane and N,3,3-dimethyl-1,5-dioxaspirol[5.5]undecan-9-yl).

The term ‘C_{x-y} cycloalkyl’ as used herein refers to a saturated hydrocarbon ring of x to y carbon atoms which can be mono, bi or tricyclic. For example, C₃₋₁₀ cycloalkyl refers to a saturated mono, bi or tricyclic hydrocarbon ring of 3 to 10 carbon atoms. Examples of C₃₋₁₀ cycloalkyl groups include cyclopropyl, cyclobutyl, cyclopentyl, cyclohexyl, cycloheptyl, cyclooctyl and adamantyl.

The term ‘aryl C_{x-y} alkyl’ as used herein refers to an aryl group as defined above attached to a C_{x-y} alkyl as defined above. For example, aryl C₁₋₆ alkyl refers to an aryl group attached to a linear or branched saturated hydrocarbon group containing from 1 to 6 carbon atoms. Examples of aryl C₁₋₆ alkyl groups include benzyl, phenylethyl, phenylpropyl, phenylbutyl, phenylpentyl and phenylhexyl.

The terms ‘heteroaryl C_{x-y} alkyl’, ‘heterocyclyl C_{x-y} alkyl’ and ‘C_{x-y} cycloalkyl C_{x-y} alkyl’ as used herein refers to a heteroaryl, heterocyclyl or C_{x-y} cycloalkyl group as defined above attached to a C_{x-y} alkyl as defined above.

The term ‘C_{x-y} alkoxy’ as used herein refers to an —O—C_{x-y} alkyl group wherein C_{x-y} alkyl is as defined above. Examples of such groups include methoxy, ethoxy, propoxy, butoxy, pentoxy and hexoxy.

The term ‘aryloxy’ as used herein refers to an —O-aryl group. Examples of such groups include phenoxy. The terms ‘heteroaryloxy’ and ‘heterocyclyloxy’ as used herein refer to an —O-heteroaryl and —O-heterocyclyl group respectively.

The term ‘halogen’ as used herein refers to a fluorine, chlorine, bromine or iodine atom, unless otherwise specified.

The term ‘C_{x-y} alkylamino’ as used herein refers to a secondary amine group (—NH(R)) of which the R group is selected from a linear or branched saturated hydrocarbon group containing from x to y carbon atoms. Examples of C_{x-y} alkylamino groups include methylamino, ethylamino and propylamino.

The term ‘C_{x-y} dialkylamino’ as used herein refers to a tertiary amine group (—NR(R*)) of which the R and R* groups are each independently selected from a linear or branched saturated hydrocarbon group containing from x to y carbon atoms. Examples of C_{x-y} dialkylamino groups include dimethylamino, methylethylamino and diethylamino.

The term ‘substituted C₁₋₆ alkyl’ used herein with reference to the identity of the various groups identified as R (for example, in the phrase ‘wherein R8e and R8f are independently selected from C₁₋₆ alkyl, substituted C₁₋₆ alkyl, aryl, heteroaryl, C₃₋₈ cycloalkyl and heterocyclyl’) means that the particular R group (e.g. R1a, R2c, R4d, R5e, etc.) can be substituted with one or more groups selected from R′, halogen, OH, OR′, SH, SR′, OCOR′, SCOR′, NH₂, NO₂, NHR′, NHSO₂NH₂, NHSO₂R′, NR′COR″, NHC(NH)NH₂, NHCOR′, NR′R″, COR′, CSR′, CN, COOH, COOR′, CONH₂, CONHOH, CONHR′, CONR′R″, CONHOR′, C(NOH)NH₂, SO₂R′, SO₃H, SO₂NH₂, SO₂NR′R″, wherein R′ and R″ are independently selected from C₁₋₆ alkyl, aryl, heteroaryl, C₃₋₈ cycloalkyl and heterocyclyl, or R′ and R″, together with the heteroatom to which they are joined, can form heterocyclyl.

‘Pharmaceutically acceptable salts’ of compounds prepared according to the present invention include salts with inorganic bases, salts with organic bases, salts with inorganic acids, salts with organic acids and salts with basic or acidic amino acids. Salts with acids may, in particular, be employed in some instances. Exemplary salts include hydrochloride salt, acetate salt, trifluoroacetate salt, methanesulfonate salt, 2-hydroxypropane-1,2,3-tricarboxylate salt, (2R,3R)-2,3-dihydroxysuccinate salt, phosphate salt and oxalate salt. The compound of the present invention may be in either solvate (e.g. hydrate) or non-solvate (e.g. non-hydrate) form. When in a solvate form, additional solvents may be alcohols such as propan-2-ol.

‘Pharmaceutically acceptable esters’ of compounds prepared according to the invention are derivatives in which one or more carboxyl (i.e. —C(O)OH) groups of the said compounds are modified by reaction with an alcoholic moiety U—OH so as to yield —C(O)OU groups, wherein U may be C₁₋₁₈ alkyl (e.g. C₁₋₆ alkyl), aryl, heteroaryl, C₃₋₈ cycloalkyl or combinations thereof.

General methods for the preparation of salts and esters are well known to the person skilled in the art. Pharmaceutical acceptability of salts and esters will depend on a variety of factors, including formulation processing characteristics and in vivo behaviour, and the skilled person would readily be able to assess such factors having regard to the present disclosure.

Where compounds prepared according to the invention exist in different enantiomeric and/or diastereoisomeric forms (including geometric isomerism about a double bond), these compounds may be prepared as isomeric mixtures or racemates, although the invention relates to all such enantiomers or isomers, whether present in an optically pure form or as mixtures with other isomers. Individual enantiomers or isomers may be obtained by methods known in the art, such as optical resolution of products or intermediates (for example chiral chromatographic separation (e.g. chiral HPLC)), or an enantiomeric synthesis approach. Similarly, where compounds prepared according to the invention may exist as alternative tautomeric forms (e.g. ketoenol, amide/imidic acid), the invention relates to preparation of the individual tautomers in isolation, and of mixtures of the tautomers in all proportions.

In particular embodiments of the process of the invention, compounds according to Formula II are prepared.

In an embodiment, when R1 and R2 together form piperidinyl in compounds having Formula I, the piperidinyl is not substituted with methyl, dimethyl, ethyl, isopropyl, tert-butyl, methoxycarbonyl, trifluoromethyl, chloro, bromo or benzyl. In another embodiment, R1 and R2 together in compounds having Formula I do not form 6,7-dimethoxy-3,4-dihydro-1H-isoquinolin-2-yl, 6-methoxy-3,4-dihydro-1H-isoquinolin-2-yl, 7-methoxy-3,4-dihydro-1H-isoquinolin-2-yl, 7-amino-3,4-dihydro-1H-isoquinolin-2-yl, 7-nitro-3,4-dihydro-1H-isoquinolin-2-yl, 3,4-dihydro-1H-isoquinolin-2-yl, 3,4-dihydro-1H-isoquinolin-1-yl, 3,4-dihydro-2H-quinolin-1-yl, pyrrolidin-1-yl, 3,6-dihydro-2H-pyridin-1-yl, 8-aza-spiro[4.5]dec-8-yl, 1,3-dihydroisoindol-2-yl, octahydroisoindol-2-yl, 1,2,6-triaza-spiro[2.5]oct-1-en-6-yl or azepan-1-yl. In a further embodiment, when R1 or R2 is methyl, the other of R1 or R2 is not 4-chlorobutyl, 4-azidobutyl, or 4-isothiocyanatobutyl. In another embodiment, Ring A in compounds having Formula I does not form a pyridine, pyrimidine, substituted pyridine or substituted pyrimidine, when R1 and R2, together with the N to which they are attached, form piperidinyl, piperazinyl, substituted piperidinyl or substituted piperazinyl. In a further embodiment, the compound prepared by the process of the invention is not (4-phenyl-1H-imidazol-1-yl)(4-(quinolin-2-ylmethyl)piperazin-1-yl)methanone.

In compounds of Formula II, zero, one or two of the atoms or groups denoted X, Y and Z can be N.

In a particular embodiment, the process of the invention is used to prepare a compound having a formula selected from Formula I or Formula II:

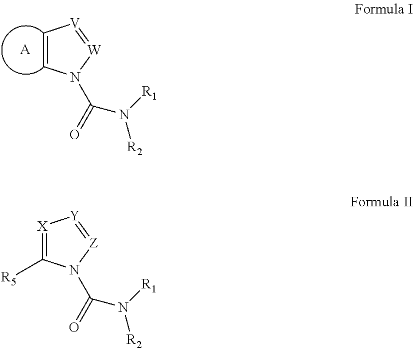

wherein:
R1 and R2 can each be independently selected from H, C₁₋₂₀ alkyl, alkoxy, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which, with the exception of H, may optionally be substituted with one or more groups selected from halogen, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, amino, C₁₋₆ alkylamino and C₁₋₆ dialkylamino, with the exception that R1 and R2 are not both H, or
R1 and R2, together with the N to which they are attached, can form a heteroaryl or heterocyclyl group, each of which may optionally be substituted with one or more groups selected from hydroxy, aryl, heteroaryl, heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, and heterocyclyloxy, each of which may optionally be substituted with a group selected from halogen, hydroxyl, C₁₋₄ alkyl, aryl, heteroaryl, C₁₋₄ alkoxy, aryloxy, heteroaryloxy, aryl C₁₋₄ alkoxy and heteroaryl C₁₋₄ alkoxy, each of which, with the exception of halogen and hydroxyl, may optionally be substituted with C₁₋₄ alkoxy;
Ring A is selected from aryl, heteroaryl and heterocyclyl moiety, each of which may optionally be substituted with one or more groups selected from halogen, hydroxyl, aryl, heteroaryl, heterocyclyl, C₁₋₆ alkoxy, aryloxy, heteroaryloxy and heterocyclyloxy, each of which, with the exception of halogen and hydroxyl, may optionally be substituted with halogen, cyano, amide and carboxylic acid;
V can be N, CH or C—R3, wherein R3 is halogen, aryl, heteroaryl, heterocyclyl or C₃₋₈ cycloalkyl, each of which, with the exception of halogen, may optionally be substituted with halogen;
W can be N, CH or C—R4, wherein R4 is C₁₋₁₀ alkyl, aryl, heteroaryl, heterocyclyl or C₃₋₈ cycloalkyl, each of which may optionally be substituted with halogen;
R5 is selected from H, C₁₋₆ alkyl, aryl, heteroaryl, heterocyclyl and C₃₋₈ cycloalkyl, each of which, with the exception of H, may optionally be substituted with halogen;
X can be N, CH or C—R6, wherein R6 is selected from C₁₋₆ alkyl, aryl, heteroaryl and heterocyclyl, each of which, with the exception of H, may optionally be substituted with one or more groups selected from halogen, hydroxyl, amine, nitro, amide, cyano, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy and heterocyclyl C₁₋₆ alkoxy;
Y can be N, CH or C—R7, wherein R7 is selected from C₁₋₆ alkyl, aryl, heteroaryl and heterocyclyl, each of which, with the exception of H, may optionally be substituted with one or more groups selected from halogen, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy and heterocyclyl C₁₋₆ alkoxy, each of which may optionally be substituted with C₁₋₄ alkyl, cyano, amine, amide, halogen, aryl, heteroaryl, heterocyclyl, aryl C₁₋₆ alkyl, heteroaryl C₁₋₆ alkyl and heterocyclyl C₁₋₆ alkyl;
Z can be N, CH or C—R8, wherein R8 is selected from C₁₋₁₀ alkyl, aryl, heteroaryl, heterocyclyl or C₃₋₃ cycloalkyl, each of which may optionally be substituted with halogen;
or a pharmaceutically acceptable salt or ester thereof;
provided that when R1 and R2 together form piperidinyl in compounds having Formula I, the piperidinyl is not substituted with methyl, dimethyl, ethyl, isopropyl, tert-butyl, trifluoromethyl, chloro, bromo or benzyl.

In an embodiment of the invention, the process is used to prepare a compound having Formula I or Formula II:

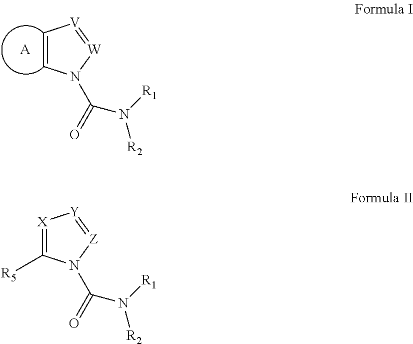

wherein:
R1 and R2 can each be independently selected from H, C₁₋₂₀ alkyl, alkoxy, aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, R1a, halogen, OH, OR1a, SH, SR1a, OCOR1a, SCOR1a, NH₂, NHR1a, NR1aR1b, COR1a, CSR1a, CN, COOH, COOR1a, CONH₂, SO₂R1a, SO₃H, SO₂NH₂, CONR1aR1b, SO₂NR1aR1b, wherein R1a and R1b are independently selected from C₁₋₆ alkyl, substituted C₁₋₆ alkyl, C₃₋₈ cycloalkyl and heterocyclyl, and R1a and R1b, together with the adjacent heteroatom, can form heterocyclyl,
wherein, when R1 or R2 is C₁₋₂₀ alkyl (such as C₁₋₆ alkyl), alkoxy, aryl, heteroaryl, heterocyclyl, C₃₋₁₀ cycloalkyl (such as C₃₋₈ cycloalkyl), aryl C₁₋₆ alkyl, heteroaryl C₁₋₆ alkyl, heterocyclyl C₁₋₆ alkyl, C₃₋₁₀ cycloalkyl C₁₋₆ alkyl, or is a group containing one or more of these moieties, each of these moieties may optionally be substituted with R1c, halogen, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₁₀ alkyl, OH, OR1c, OCOR1c, SH, SR1c, SCOR1c, NH₂, NHR1c, NR1cR1d, COR1c, CSR1c, CN, COOH, COOR1c, CONH₂, SO₂R1c, SO₃H, SO₂NH₂, CONR1cR1d, SO₂NR1cR1d, wherein R1c and R1 d are independently selected from C₁₋₆ alkyl, substituted C₁₋₆ alkyl, C₃₋₈ cycloalkyl and heterocyclyl, and R1c and R1d, together with the adjacent heteroatom, can form heterocyclyl,
wherein, when the substituent of R1 or R2 is C₁₋₁₀ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₆ alkyl, C₃₋₈ cycloalkyl or is a group containing one or more of these moieties, each of these moieties may optionally be substituted with R1e, C₁₋₁₀ alkyl, OH, OR1e, OCOR1e, SH, SR1e, SCOR1e, NH₂, NHR1e, NR1eR1f, COR1e, CSR1e, CN, COOH, COOR1e, CONH₂, SO₂R1e, SO₃H, SO₂NH₂, CONR1eR1f, SO₂NR1eR1f, wherein R1e and R1f are independently selected from C₁₋₆ alkyl, substituted C₁₋₆ alkyl, C₃₋₈ cycloalkyl and heterocyclyl, and R1e and R1f, together with the adjacent heteroatom, can form heterocyclyl, with the exception that R1 and R2 are not both H, or
R1 and R2, together with the N to which they are attached, can form a heteroaryl or heterocyclyl group, each of which may optionally be substituted with one or more groups selected from hydroxy, aryl, heteroaryl, partially or fully saturated heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, R2a, halogen, OH, OR2a, SH, SR2a, OCOR2a, SCOR2a, NH₂, NHR2a, NR2aR2b, COR2a, CSR2a, CN, COOH, COOR2a, CONH₂, SO₂R2a, SO₃H, SO₂NH₂, CONR2aR2b, SO₂NR2aR2b, wherein R2a and R2b are independently selected from C₁₋₆ alkyl, substituted C₁₋₆ alkyl, C₃₋₈ cycloalkyl and heterocyclyl, and R2a and R2b, together with the adjacent heteroatom, can form heterocyclyl,
wherein, when the substituent of the heteroaryl or heterocyclyl formed by R1 and R2 together is aryl, heteroaryl, heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, or a group containing one or more of these moieties, each of these moieties may optionally be substituted with a group selected from halogen, hydroxyl, C₁₋₆ alkyl, aryl, heteroaryl, heterocyclyl, C₃₋₈ cycloalkyl, C₁₋₄ alkoxy, aryloxy, heteroaryloxy, heterocyclyloxy, C₃₋₈ cycloalkyloxy, aryl C₁₋₄ alkoxy, heteroaryl C₁₋₄ alkoxy, heterocyclyl C₁₋₄ alkoxy, C₃₋₈ cycloalkyl C₁₋₄ alkoxy, R2c, OR2c, SH, SR2c, OCOR2c, SCOR2c, NH₂, NHR2c, NR2cR2d, COR2c, CSR2c, CN, COOH, COOR2c, CONH₂, SO₂R2c, SO₃H, SO₂NH₂, CONR2cR2d, SO₂NR2cR2d, wherein R2c and R2d are independently selected from C₁₋₆ alkyl, substituted C₁₋₆ alkyl, C₃₋₈ cycloalkyl and heterocyclyl, and R2c and R2d, together with the adjacent heteroatom, can form heterocyclyl,
wherein, when the substituent of the substituent of the heteroaryl or heterocyclyl of R1 and R2 together is C₁₋₄ alkyl, aryl, heteroaryl, heterocyclyl, C₃₋₈ cycloalkyl, C₁₋₄ alkoxy, aryloxy, heteroaryloxy, heterocyclyloxy, C₃₋₈ cycloalkyloxy, aryl C₁₋₄ alkoxy, heteroaryl C₁₋₄ alkoxy, heterocyclyl C₁₋₄ alkoxy, C₃₋₈ cycloalkyl C₁₋₄ alkoxy, or is a group containing one or more of these moieties, each of these moieties may optionally be substituted with C₁₋₄ alkoxy, R2e, halogen, OH, OR2e, SH, SR2e, OCOR2e, SCOR2e, NH₂, NHR2e, NR2eR2f, COR2e, CSR2e, CN, COOH, COOR2e, CONH₂, SO₂R2e, SO₃H, SO₂NH₂, CONR2eR2f, SO₂NR2eR2f, wherein R2e and R2f are independently selected from C₁₋₆ alkyl, substituted C₁₋₆ alkyl, C₃₋₈ cycloalkyl and heterocyclyl, and R2e and R2f, together with the adjacent heteroatom, can form heterocyclyl;
Ring A is selected from aryl, heteroaryl and heterocyclyl moiety, each of which may optionally be substituted with one or more groups selected from halogen, C₁₋₆ alkyl, hydroxyl, aryl, heteroaryl, heterocyclyl, C₁₋₆ alkoxy, aryloxy, heteroaryloxy, heterocyclyloxy, Ra, C₁₋₁₀ alkyl, OH, ORa, OCORa, SH, SRa, SCORa, NH₂, NHRa, NRaRb, CORa, CSRa, CN, COOH, COORa, CONH₂, SO₂Ra, SO₃H, SO₂NH₂, CONRaRb, SO₂NRaRb, wherein Ra and Rb are independently selected from C₁₋₆ alkyl, substituted C₁₋₆ alkyl, C₃₋₈ cycloalkyl and heterocyclyl, and Ra and Rb, together with the adjacent heteroatom, can form heterocyclyl,
wherein, when Ring A is substituted with C₁₋₆ alkyl, aryl, heteroaryl, heterocyclyl, C₁₋₆ alkoxy, aryloxy, heteroaryloxy, heterocyclyloxy, C₁₋₁₀ alkyl, C₃₋₈ cycloalkyl or is substituted with a group containing one or more of these moieties, each of these moieties may optionally be substituted with Rc, C₁₋₁₀ alkyl, OH, ORc, OCORc, SH, SRc, SCORc, NH₂, NHRc, NRcRd, CORc, CSRc, CN, COOH, COORc, CONH₂, SO₂Rc, SO₃H, SO₂NH₂, CONRcRd, SO₂NRcRd, wherein Rc and Rd are independently selected from C₁₋₆ alkyl, substituted C₁₋₆ alkyl, C₃₋₈ cycloalkyl and heterocyclyl, and Rc and Rd, together with the adjacent heteroatom, can form heterocyclyl;
V can be N, CH or C—R3, wherein R3 is halogen, C₁₋₁₀ alkyl, aryl, heteroaryl, heterocyclyl, C₃₋₈ cycloalkyl, C₁₋₆ alkoxy, aryloxy, heteroaryloxy, heterocyclyloxy, R3a, OH, OR3a, SH, SR3a, OCOR3a, SCOR3a, NH₂, NHR3a, NR3aR3b, COR3a, CSR3a, CN, COOH, COOR3a, CONH₂, SO₂R3a, SO₃H, SO₂NH₂, CONR3aR3b, SO₂NR3aR3b, wherein R3a and R3b are independently selected from C₁₋₆ alkyl, substituted C₁₋₆ alkyl, C₃₋₈ cycloalkyl and heterocyclyl, and R3a and R3b, together with the adjacent heteroatom, can form heterocyclyl,
wherein, when R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with aryl, heteroaryl, heterocyclyl, C₁₋₆ alkoxy, aryloxy, heteroaryloxy, heterocyclyloxy, R3c, C₁₋₁₀ alkyl, OH, OR3c, OCOR3c, SH, SR3c, SCOR3c, NH₂, NHR3c, NR3cR3d, COR3c, CSR3c, CN, COOH, COOR3c, CONH₂, SO₂R3c, SO₃H, SO₂NH₂, CONR3cR3d, SO₂NR3cR3d, wherein R3c and R3d are independently selected from C₁₋₆ alkyl, substituted C₁₋₆ alkyl, C₃₋₈ cycloalkyl and heterocyclyl, and R3c and R3d, together with the adjacent heteroatom, can form heterocyclyl,
wherein, when the substituent of R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with R3e, C₁₋₁₀ alkyl, OH, OR3e, OCOR3e, SH, SR3e, SCOR3e, NH₂, NHR3e, NR3eR3f, COR3e, CSR3e, CN, COOH, COOR3e, CONH₂, SO₂R3e, SO₃H, SO₂NH₂, CONR3eR3f, SO₂NR3eR3f, wherein R3e and R3f are independently selected from C₁₋₆ alkyl, substituted C₁₋₆ alkyl, C₃₋₈ cycloalkyl and heterocyclyl, and R3e and R3f, together with the adjacent heteroatom, can form heterocyclyl;
W can be N, CH or C—R4, wherein R4 is halogen, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, R4a, OH, OR4a, SH, SR4a, OCOR4a, SCOR4a, NH₂, NHR4a, NR4aR4b, COR4a, CSR4a, CN, COOH, COOR4a, CONH₂, SO₂R4a, SO₃H, SO₂NH₂, CONR4aR4b, SO₂NR4aR4b, wherein R4a and R4b are independently selected from C₁₋₆ alkyl, substituted C₁₋₈ alkyl, C₃₋₈ cycloalkyl and heterocyclyl, and R4a and R4b, together with the adjacent heteroatom, can form heterocyclyl,
wherein, when R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with aryl, heteroaryl, heterocyclyl, C₁₋₆ alkoxy, aryloxy, heteroaryloxy, heterocyclyloxy, R4c, C₁₋₁₀ alkyl, OH, OR4c, OCOR4c, SH, SR4c, SCOR4c, NH₂, NHR4c, NR4cR4d, COR4c, CSR4c, CN, COOH, COOR4c, CONH₂, SO₂R4c, SO₃H, SO₂NH₂, CONR4cR4d, SO₂NR4cR4d, wherein R4c and R4d are independently selected from C₁₋₆ alkyl, substituted C₁₋₆ alkyl, C₃₋₈ cycloalkyl and heterocyclyl, and R4c and R4d, together with the adjacent heteroatom, can form heterocyclyl,
wherein, when the substituent of R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with R4e, C₁₋₁₀ alkyl, OH, OR4e, OCOR4e, SH, SR4e, SCOR4e, NH₂, NHR4e, NR4eR4f, COR4e, CSR4e, CN, COOH, COOR4e, CONH₂, SO₂R4e, SO₃H, SO₂NH₂, CONR4eR4f, SO₂NR4eR4f, wherein R4e and R4f are independently selected from C₁₋₆ alkyl, substituted C₁₋₆ alkyl, C₃₋₈ cycloalkyl and heterocyclyl, and R4e and R4f, together with the adjacent heteroatom, can form heterocyclyl;
R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NHR5a, NR5aR5b, COR5a, CSR5a, CN, COOH, COOR5a, CONH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, C₃₋₈ cycloalkyl and heterocyclyl, and R5a and R5b, together with the adjacent heteroatom, can form heterocyclyl,
wherein, when R5 is C₁₋₆ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with aryl, heteroaryl, heterocyclyl, C₁₋₆ alkoxy, aryloxy, heteroaryloxy, heterocyclyloxy, R5c, C₁₋₆ alkyl, OH, OR5c, OCOR5c, SH, SR5c, SCOR5c, NH₂, NHR5c, NR5cR5d, COR5c, CSR5c, CN, COOH, COOR5c, CONH₂, SO₂R5c, SO₃H, SO₂NH₂, CONR5cR5d, SO₂NR5cR5d, wherein R5c and R5d are independently selected from C₁₋₆ alkyl, substituted C₁₋₆ alkyl, C₃₋₈ cycloalkyl and heterocyclyl, and R5c and R5d, together with the adjacent heteroatom, can form heterocyclyl,
wherein, when the substituent of R5 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with R5e, C₁₋₆ alkyl, OH, OR5e, OCOR5e, SH, SR5e, SCOR5e, NH₂, NHR5e, NR5eR5f, COR5e, CSR5e, CN, COOH, COOR5e, CONH₂, SO₂R5e, SO₃H, SO₂NH₂, CONR5eR5f, SO₂NR5eR5f, wherein R5e and R5f are independently selected from C₁₋₆ alkyl, substituted C₁₋₆ alkyl, C₃₋₈ cycloalkyl and heterocyclyl, and R5e and R5f, together with the adjacent heteroatom, can form heterocyclyl;
X can be N, CH or C—R6, wherein R6 is selected from C₁₋₆ alkyl, aryl, heteroaryl, heterocyclyl, C₁₋₆ alkoxy, aryloxy, heteroaryloxy, heterocyclyloxy, R6a, halogen, OH, OR6a, SH, SR6a, OCOR6a, SCOR6a, NH₂, NHR6a, NR6aR6b, COR6a, CSR6a, CN, COOH, COOR6a, CONH₂, SO₂R6a, SO₃H, SO₂NH₂, CONR6aR6b, SO₂NR6aR6b, wherein R6a and R6b are independently selected from C₁₋₆ alkyl, substituted C₁₋₆ alkyl, C₃₋₈ cycloalkyl and heterocyclyl, and R6a and R6b, together with the adjacent heteroatom, can form heterocyclyl,
wherein, when R6 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with R6c, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR6c, OCOR6c, SH, SR6c, SCOR6c, NO₂, NH₂, NHR6c, NR6cR6d, COR6c, CSR6c, CN, COOH, COOR6c, CONH₂, SO₂R6e, SO₃H, SO₂NH₂, CONR6cR6d, SO₂NR6cR6d, wherein R6c and R6d are independently selected from C₁₋₆ alkyl, substituted C₁₋₆ alkyl, C₃₋₈ cycloalkyl and heterocyclyl, and R6c and R6d, together with the adjacent heteroatom, can form heterocyclyl,
wherein, when the substituent of R6 is C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with R6e, C₁₋₆ alkyl, OH, OR6e, OCOR6e, SH, SR6e, SCOR6e, NH₂, NHR6e, NR6eR6f, COR6e, CSR6e, CN, COOH, COOR6e, CONH₂, SO₂R6e, SO₃H, SO₂NH₂, CONR6eR6f, SO₂NR6eR6f, wherein R6e and R6f are independently selected from C₁₋₆ alkyl, substituted C₁₋₆ alkyl, C₃₋₈ cycloalkyl and heterocyclyl, and R6e and R6f, together with the adjacent heteroatom, can form heterocyclyl;
Y can be N, CH or C—R7, wherein R7 is selected from C₁₋₆ alkyl, aryl, heteroaryl, heterocyclyl, C₁₋₆ alkoxy, aryloxy, heteroaryloxy, heterocyclyloxy, R7a, halogen, OH, OR7a, SH, SR7a, OCOR7a, SCOR7a, NH₂, NHR7a, NR7aR7b, COR7a, CSR7a, CN, COOH, COOR7a, CONH₂, SO₂R7a, SO₃H, SO₂NH₂, CONR7aR7b, SO₂NR7aR7b, wherein R7a and R7b are independently selected from C₁₋₆ alkyl, substituted C₁₋₆ alkyl, C₃₋₆ cycloalkyl and heterocyclyl, and R7a and R7b, together with the adjacent heteroatom, can form heterocyclyl,
wherein, when R7 is C₁₋₆ alkyl, aryl, heteroaryl, heterocyclyl, C₁₋₆ alkoxy, aryloxy, heteroaryloxy, heterocyclyloxy, C₃₋₈ cycloalkyl or is a group containing one or more of these moieties, each of these moieties may optionally be substituted with R7c, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, 07c, OCOR7c, SH, SR7c, SCOR7c, NH₂, NHR7c, NR7cR7d, COR7c, CSR7c, CN, COOH, COOR7c, CONH₂, SO₂R7c, SO₃H, SO₂NH₂, CONR7cR7d, SO₂NR7cR7d, wherein R7c and R7d are independently selected from C₁₋₆ alkyl, substituted C₁₋₆ alkyl, C₃₋₈ cycloalkyl and heterocyclyl, and R7c and R7d, together with the adjacent heteroatom, can form heterocyclyl,
wherein, when the substituent of R7 is C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with R7e, C₁₋₆ alkyl, OH, OR7e, OCOR7e, SH, SR7e, SCOR7e, NH₂, NHR7e, NR7eR7f, COR7e, CSR7e, CN, COOH, COOR7e, CONH₂, SO₂R7e, SO₃H, SO₂NH₂, CONR7eR7f, SO₂NR7eR7f, wherein R7e and R7f are independently selected from C₁₋₆ alkyl, substituted C₁₋₆ alkyl, C₃₋₈ cycloalkyl and heterocyclyl, and R7e and R7f, together with the adjacent heteroatom, can form heterocyclyl;
Z can be N, CH or C—R8, wherein R8 is selected from C₁₋₆ alkyl, aryl, heteroaryl, heterocyclyl, C₁₋₆ alkoxy, aryloxy, heteroaryloxy, heterocyclyloxy, R8a, halogen, OH, OR8a, SH, SR8a, OCOR8a, SCOR8a, NH₂, NHR8a, NR8aR8b, COR8a, CSR8a, CN, COOH, COOR5a, CONH₂, SO₂R8a, SO₃H, SO₂NH₂, CONR8aR8b, SO₂NR8aR8b, wherein R8a and R8b are independently selected from C₁₋₆ alkyl, substituted C₁₋₆ alkyl, C₃₋₈ cycloalkyl and heterocyclyl, and R8a and R8b, together with the adjacent heteroatom, can form heterocyclyl,
wherein, when R8 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with R8c, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR5c, OCOR8c, SH, SR8c, SCOR8c, NH₂, NHR8c, NR8cR8d, COR8c, CSR8c, CN, COOH, COOR8c, CONH₂, SO₂R8c, SO₃H, SO₂NH₂, CONR8cR8d, SO₂NR8cR8d, wherein R8c and R8d are independently selected from C₁₋₆ alkyl, substituted C₁₋₆ alkyl, C₃₋₈ cycloalkyl and heterocyclyl, and R8c and R8d, together with the adjacent heteroatom, can form heterocyclyl,
wherein, when the substituent of R8 is C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with R8e, C₁₋₆ alkyl, OH, OR8e, OCOR8e, SH, SR8e, SCOR8e, NH₂, NHR8e, NR8eR8f, COR8e, CSR8e, CN, COOH, COOR8e, CONH₂, SO₂R8e, SO₃H, SO₂NH₂, CONR8eR8f, SO₂NR8eR8f, wherein R8e and R8f are independently selected from C₁₋₆ alkyl, substituted C₁₋₆ alkyl, C₃₋₈ cycloalkyl and heterocyclyl, and R8e and R8f, together with the adjacent heteroatom, can form heterocyclyl;
or a pharmaceutically acceptable salt or ester thereof.

In such an embodiment, the compound may be limited by the following exceptions:

provided that when R1 and R2 together form piperidinyl in compounds having Formula I, the piperidinyl is not substituted with methyl, dimethyl, ethyl, isopropyl, tert-butyl, trifluoromethyl, chloro, bromo or benzyl,
provided that R1 and R2 together in compounds having Formula I do not form 6,7-dimethoxy-3,4-dihydro-1H-isoquinolin-2-yl, 6-methoxy-3,4-dihydro-1H-isoquinolin-2-yl, 7-methoxy-3,4-dihydro-1H-isoquinolin-2-yl, 7-amino-3,4-dihydro-1H-isoquinolin-2-yl, 7-nitro-3,4-dihydro-1H-isoquinolin-2-yl, 3,4-dihydro-1H-isoquinolin-2-yl, 3,4-dihydro-1H-isoquinolin-1-yl, 3,4-dihydro-2H-quinolin-1-yl, pyrrolidin-1-yl, 3,6-dihydro-2H-pyridin-1-yl, 8-aza-spiro[4.5]dec-8-yl, 1,3-dihydroisoindol-2-yl, octahydroisoindol-2-yl, 1,2,6-triaza-spiro[2.5]oct-1-en-6-yl or azepan-1-yl, and/or
provided that Ring A in compounds having Formula I does not form a pyridine, pyrazine, substituted pyridine or substituted pyrazine, when R1 and R2, together with the N to which they are attached, form piperidinyl, piperazinyl, substituted piperidinyl or substituted piperazinyl.

In accordance with a further embodiment of the invention, the process is used for preparing a compound having Formula I or Formula II:

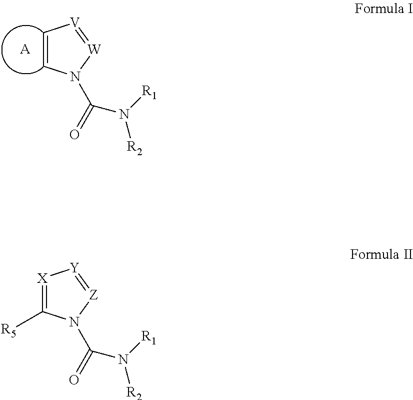

wherein:
R1 and R2 can each be independently selected from H, C₁₋₂₀ alkyl, C₁₋₆ alkoxy, aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, R1a, halogen, OH, OR1a, SH, SR1a, OCOR1a, SCOR1a, NH₂, NHR1a, NR1aR1b, COR1a, CSR1a, CN, COOH, COOR1a, CONH₂, SO₂R1a, SO₃H, SO₂NH₂, CONR1aR1b, SO₂NR1aR1b, wherein R1a and R1b are independently selected from C₁₋₆ alkyl, substituted C₁₋₆ alkyl, aryl, heteroaryl, C₃₋₈ cycloalkyl and heterocyclyl, or R1a and R1b, together with the heteroatom to which they are joined, can form heterocyclyl,
wherein, when R1 or R2 is C₁₋₂₀ alkyl (such as C₁₋₆ alkyl), alkoxy, aryl, heteroaryl, heterocyclyl, C₃₋₁₀ cycloalkyl (such as C₃₋₈ cycloalkyl), aryl C₁₋₆ alkyl, heteroaryl C₁₋₆ alkyl, heterocyclyl C₁₋₆ alkyl, C₃₋₁₀ cycloalkyl C₁₋₆ alkyl, or is a group containing one or more of these moieties, each of these moieties may optionally be substituted with one or more groups selected from R1c, halogen, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₁₀ alkyl, OH, OR1c, OCOR1c, SH, SR1c, SCOR1c, NH₂, NO₂, NHR1c, NR1cR1d, COR1c, CSR1c, CN, COOH, COOR1c, CONH₂, SO₂R1c, SO₃H, SO₂NH₂, CONR1cR1d, SO₂NR1cR1d, wherein R1c and R1d are independently selected from C₁₋₆ alkyl, substituted C₁₋₆ alkyl, aryl, heteroaryl, C₃₋₈ cycloalkyl and heterocyclyl, or R1c and R1d, together with the heteroatom to which they are joined, can form heterocyclyl,
wherein, when the substituent of R1 or R2 is C₁₋₁₀ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₆ alkyl, C₃₋₆ cycloalkyl or is a group containing one or more of these moieties, each of these moieties may optionally be substituted with one or more groups selected from R1e, halogen, C₁₋₁₀ alkyl, OH, OR1e, OCOR1e, SH, SR1e, SCOR1e, NH₂, NO₂, NHR1e, NR1eR1f, COR1e, CSR1e, CN, COOH, COOR1e, CONH₂, SO₂R1e, SO₃H, SO₂NH₂, CONR1eR1f, SO₂NR1eR1f, wherein R1e and R1 f are independently selected from C₁₋₆ alkyl, substituted C₁₋₆ alkyl, aryl, heteroaryl, C₃₋₈ cycloalkyl and heterocyclyl, or R1e and R1f, together with the heteroatom to which they are joined, can form heterocyclyl,
with the exception that R1 and R2 are not both H,
or
R1 and R2, together with the N to which they are attached, can form a heteroaryl or heterocyclyl group, each of which may optionally be substituted with one or more oxygen atoms or one or more groups selected from hydroxy, aryl, heteroaryl, partially or fully saturated heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, R2a, halogen, OH, OR2a, SH, SR2a, OCOR2a, SCOR2a, NH₂, NO₂, NHR2a, NR2aR2b, COR2a, CSR2a, CN, COOH, COOR2a, CONH₂, SO₂R2a, SO₃H, SO₂NH₂, CONR2aR2b, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of the heteroaryl or heterocyclyl formed by R1 and R2 together is aryl, heteroaryl, heterocyclyl, C₃₋₃ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, or a group containing one or more of these moieties, each of these moieties may optionally be substituted with one or more groups selected from halogen, hydroxyl, C₁₋₆ alkyl, aryl, heteroaryl, heterocyclyl, C₃₋₈ cycloalkyl, C₁₋₄ alkoxy, aryloxy, heteroaryloxy, heterocyclyloxy, C₃₋₈ cycloalkyloxy, aryl C₁₋₄ alkoxy, heteroaryl C₁₋₆ alkoxy, heterocyclyl C₁₋₄ alkoxy, C₃₋₈ cycloalkyl C₁₋₄ alkoxy, R2c, OR2c, SH, SR2c, OCOR2c, SCOR2c, NH₂, NO₂, NHR2c, NR2cR2d, COR2c, CSR2c, CN, COOH, COOR2c, CONH₂, SO₂R2c, SO₃H, SO₂NH₂, CONR2cR2d, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
wherein, when the substituent of the substituent of the heteroaryl or heterocyclyl formed by R1 and R2 together is C₁₋₆ alkyl, aryl, heteroaryl, heterocyclyl, C₃₋₈ cycloalkyl, C₁₋₆ alkoxy, aryloxy, heteroaryloxy, heterocyclyloxy, C₃₋₈ cycloalkyloxy, aryl C₁₋₄ alkoxy, heteroaryl C₁₋₄ alkoxy, heterocyclyl C₁₋₄ alkoxy, C₃₋₈ cycloalkyl C₁₋₄ alkoxy, or is a group containing one or more of these moieties, each of these moieties may optionally be substituted with one or more groups selected from C₁₋₄ alkoxy, R2e, halogen, OH, OR2e, SH, SR2e, OCOR2e, SCOR2e, NH₂, NO₂, NHR2e, NR2eR2f, NHCOR2e, COR2e, CSR2e, CN, COOH, COOR2e, CONH₂, SO₂R2e, SO₃H, SO₂NH₂, CONR2eR2f, SO₂NR2eR2f, wherein R2e and R2f are independently selected from C₁₋₆ alkyl, substituted C₁₋₆ alkyl, aryl, heteroaryl, C₃₋₈ cycloalkyl and heterocyclyl, or R2e and R2f, together with the heteroatom to which they are joined, can form heterocyclyl;
Ring A is selected from aryl, heteroaryl and heterocyclyl moieties, each of which may optionally be substituted with one or more groups selected from halogen, C₁₋₆ alkyl, hydroxyl, aryl, heteroaryl, heterocyclyl, C₁₋₆ alkoxy, aryloxy, heteroaryloxy, heterocyclyloxy, Ra, C₁₋₁₀ alkyl, OH, ORa, OCORa, SH, SRa, SCORa, NH₂, NO₂, NHRa, NRaRb, CORa, CSRa, CN, COOH, COORa, CONH₂, CONHOH, CONHORa, SO₂Ra, SO₃H, SO₂NH₂, CONRaRb, SO₂NRaRb, wherein Ra and Rb are independently selected from C₁₋₆ alkyl, substituted C₁₋₆ alkyl, aryl, heteroaryl, C₃₋₈ cycloalkyl and heterocyclyl, or Ra and Rb, together with the heteroatom to which they are joined, can form heterocyclyl,
wherein, when Ring A is substituted with C₁₋₆ alkyl, aryl, heteroaryl, heterocyclyl, C₁₋₆ alkoxy, aryloxy, heteroaryloxy, heterocyclyloxy, C₁₋₁₀ alkyl, C₃₋₈ cycloalkyl or is substituted with a group containing one or more of these moieties, each of these moieties may optionally be substituted with one or more groups selected from halogen, Rc, C₁₋₁₀ alkyl, aryl C₁₋₆ alkyl, OH, ORc, OCORc, SH, SRc, SCORc, NH₂, NO₂, NHRc, NRcRd, CORc, CSRc, CN, COOH, COORc, CONH₂, SO₂Rc, SO₃H, SO₂NH₂, CONRcRd, SO₂NRcRd, wherein Rc and Rd are independently selected from C₁₋₆ alkyl, substituted C₁₋₆ alkyl, aryl, heteroaryl, C₃₋₈ cycloalkyl and heterocyclyl, or Rc and Rd, together with the heteroatom to which they are joined, can form heterocyclyl;
V can be N, CH or C—R3, wherein R3 is halogen, C₁₋₁₀ alkyl, aryl, heteroaryl, heterocyclyl, C₃₋₈ cycloalkyl, C₁₋₆ alkoxy, aryloxy, heteroaryloxy, heterocyclyloxy, R3a, OH, OR3a, SH, SR3a, OCOR3a, SCOR3a, NH₂, NO₂, NHR3a, NR3aR3b, COR3a, CSR3a, CN, COOH, COOR3a, CONH₂, SO₂R3a, SO₃H, SO₂NH₂, CONR3aR3b, SO₂NR3aR3b, wherein R3a and R3b are independently selected from C₁₋₆ alkyl, substituted C₁₋₆ alkyl, aryl, heteroaryl, C₃₋₈ cycloalkyl and heterocyclyl, or R3a and R3b, together with the heteroatom to which they are joined, can form heterocyclyl,
wherein, when R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R3c, C₁₋₁₀ alkyl, OH, OR3c, OCOR3c, SH, SR3c, SCOR3c, NH₂, NO₂, NHR3c, NR3cR3d, COR3c, CSR3c, CN, COOH, COOR3c, CONH₂, SO₂R3c, SO₃H, SO₂NH₂, CONR3cR3d, SO₂NR3cR3d, wherein R3c and R3d are independently selected from C₁₋₆ alkyl, substituted C₁₋₆ alkyl, aryl, heteroaryl, C₃₋₈ cycloalkyl and heterocyclyl, or R3c and R3d, together with the heteroatom to which they are joined, can form heterocyclyl,
wherein, when the substituent of R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, R3e, C₁₋₁₀ alkyl, OH, OR3e, OCOR3e, SH, SR3e, SCOR3e, NH₂, NO₂, NHR3e, NR3eR3f, COR3e, CSR3e, CN, COOH, COOR3e, CONH₂, SO₂R3e, SO₃H, SO₂NH₂, CONR3eR3f, SO₂NR3eR3f, wherein R3e and R1f are independently selected from C₁₋₆ alkyl, substituted C₁₋₆ alkyl, aryl, heteroaryl, C₃₋₈ cycloalkyl and heterocyclyl, or R3e and R3f, together with the heteroatom to which they are joined, can form heterocyclyl;
W can be N, CH or C—R4, wherein R4 is halogen, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, R4a, OH, OR4a, SH, SR4a, OCOR4a, SCOR4a, NH₂, NO₂, NHR4a, NR4aR4b, COR4a, CSR4a, CN, COOH, COOR4a, CONH₂, SO₂R4a, SO₃H, SO₂NH₂, CONR4aR4b, SO₂NR4aR4b, wherein R4a and R4b are independently selected from C₁₋₆ alkyl, substituted C₁₋₆ alkyl, aryl, heteroaryl, C₃₋₈ cycloalkyl and heterocyclyl, or R4a and R4b, together with the heteroatom to which they are joined, can form heterocyclyl,
wherein, when R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R4c, C₁₋₁₀ alkyl, OH, OR4c, OCOR4c, SH, SR4c, SCOR4c, NH₂, NO₂, NHR4c, NR4cR4d, COR4c, CSR4c, CN, COOH, COOR4c, CONH₂, SO₂R4c, SO₃H, SO₂NH₂, CONR4cR4d, SO₂NR4cR4d, wherein R4c and R4d are independently selected from C₁₋₆ alkyl, substituted C₁₋₆ alkyl, aryl, heteroaryl, C₃₋₈ cycloalkyl and heterocyclyl, or R4c and R4d, together with the heteroatom to which they are joined, can form heterocyclyl,
wherein, when the substituent of R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈; cycloalkyl, or is a group containing one or more of these moieties, each of these moieties may optionally be substituted with one or more groups selected from halogen, R4e, C₁₋₁₀ alkyl, OH, OR4e, OCOR4e, SH, SR4e, SCOR4e, NH₂, NO₂, NHR4e, NR4eR4f, COR4e, CSR4e, CN, COOH, COOR4e, CONH₂, SO₂R4e, SO₃H, SO₂NH₂, CONR4eR4f, SO₂NR4eR4f, wherein R4e and R4f are independently selected from C₁₋₆ alkyl, substituted C₁₋₆ alkyl, aryl, heteroaryl, C₃₋₈ cycloalkyl and heterocyclyl, or R4e and R4f, together with the heteroatom to which they are joined, can form heterocyclyl;
R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NO₂, NHR5a, NR5aR5b, COR5a, CSR5a, CN, COOH, COOR5a, CONH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, aryl, heteroaryl, C₃₋₆ cycloalkyl and heterocyclyl, or R5a and R5b, together with the heteroatom to which they are joined, can form heterocyclyl,
wherein, when R5 is C₁₋₆ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R5c, C₁₋₆ alkyl, OH, OR5c, OCOR5c, SH, SR5c, SCOR5c, NH₂, NO₂, NHR5c, NR5cR5d, COR5c, CSR5c, CN, COOH, COOR5c, CONH₂, SO₂R5c, SO₃H, SO₂NH₂, CONR5cR5d, SO₂NR5cR5d, wherein R5c and R5d are independently selected from C₁₋₆ alkyl, substituted C₁₋₆ alkyl, aryl, heteroaryl, C₃₋₈ cycloalkyl and heterocyclyl, or R5c and R5d, together with the heteroatom to which they are joined, can form heterocyclyl,
wherein, when the substituent of R5 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R5e, C₁₋₆ alkyl, OH, OR5e, OCOR5e, SH, SR5e, SCOR5e, NH₂, NO₂, NHR5e, NR5eR5f, COR5e, CSR5e, CN, COOH, COOR5e, CONH₂, SO₂R5e, SO₃H, SO₂NH₂, CONR5eR5f, SO₂NR5eR5f, wherein R5e and R5f are independently selected from C₁₋₆ alkyl, substituted C₁₋₆ alkyl, aryl, heteroaryl, C₃₋₈ cycloalkyl and heterocyclyl, or R5e and R5f, together with the heteroatom to which they are joined, can form heterocyclyl;
X can be N, CH or C—R6, wherein R6 is selected from C₁₋₆ alkyl, aryl, heteroaryl, heterocyclyl, C₁₋₆ alkoxy, aryloxy, heteroaryloxy, heterocyclyloxy, R6a, halogen, OH, OR6a, SH, SR6a, OCOR6a, SCOR6a, NH₂, NO₂, NHR6a, NR6aR6b, COR6a, CSR6a, CN, COOH, COOR6a, CONH₂, SO₂R6a, SO₃H, SO₂NH₂, CONR6aR6b, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl,
wherein, when R6 is heteroaryl or heterocyclyl, each of these moieties may optionally be substituted with one or more oxygen atoms, and when R6 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R6c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR6c, OCOR6c, SH, SR6c, SCOR6c, NH₂, NO₂, NHR6c, NR6cR6d, COR6c, CSR6c, CN, COOH, COOR6c, CONH₂, CONHOH, C(NOH)NH₂, SO₂R6c, SO₃H, SO₂NH₂, CONR6cR6d, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R6c and R6d, together with the heteroatom to which they are joined, can form heterocyclyl,
wherein, when the substituent of R6 is heteroaryl or heterocyclyl, each of these moieties may optionally be substituted with one or more oxygen atoms, or when the substituent of R6 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R6e, C₁₋₆ alkyl, C₁₋₄ alkoxy, OH, OR6e, OCOR6e, SH, SR6e, SCOR6e, NH₂, NO₂, NHR6e, NR6eR6f, COR6e, CSR6e, CN, COOH, COOR6e, CONH₂, C(NOH)NH₂, SO₂R6e, SO₃H, SO₂NH₂, CONR6eR6f, SO₂NR6eR6f, wherein R6e and R6f are independently selected from C₁₋₆ alkyl, substituted C₁₋₆ alkyl, aryl, heteroaryl, C₃₋₈ cycloalkyl and heterocyclyl, or R6e and R6f, together with the heteroatom to which they are joined, can form heterocyclyl;
Y can be N, CH or C—R7, wherein R7 is selected from C₁₋₆ alkyl, aryl, heteroaryl, heterocyclyl, C₁₋₆ alkoxy, aryloxy, heteroaryloxy, heterocyclyloxy, R7a, halogen, OH, OR7a, SH, SR7a, OCOR7a, SCOR7a, NH₂, NO₂, NHR7a, NR7aR7b, COR7a, CSR7a, CN, COOH, COOR7a, CONH₂, SO₂R7a, SO₃H, SO₂NH₂, CONR7aR7b, SO₂NR7aR7b, wherein R7a and R7b are independently selected from C₁₋₆ alkyl, substituted C₁₋₆ alkyl, aryl, heteroaryl, C₃₋₈ cycloalkyl and heterocyclyl, or R7a and R7b, together with the heteroatom to which they are joined, can form heterocyclyl,
wherein, when R7 is heteroaryl or heterocyclyl, each of these moieties may optionally be substituted with one or more oxygen atoms, and when R7 is C₁₋₆ alkyl, aryl, heteroaryl, heterocyclyl, C₁₋₆ alkoxy, aryloxy, heteroaryloxy, heterocyclyloxy, C₃₋₈ cycloalkyl or is a group containing one or more of these moieties, each of these moieties may optionally be substituted with one or more groups selected from halogen, R7c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, 07c, OCOR7c, SH, SR7c, SCOR7c, NH₂, NO₂, NHR7c, NR7cR7d, COR7c, CSR7c, CN, COOH, COOR7c, CONH₂, CONHOH, C(NOH)NH₂, SO₂R7c, SO₃H, SO₂NH₂, CONR7cR7d, SO₂NR7cR7d, wherein R7c and R7d are independently selected from C₁₋₆ alkyl, substituted C₁₋₆ alkyl, aryl, heteroaryl, C₃₋₈ cycloalkyl and heterocyclyl, or R7c and R7d, together with the heteroatom to which they are joined, can form heterocyclyl,
wherein, when the substituent of R7 is heteroaryl or heterocyclyl, each of these moieties may optionally be substituted with one or more oxygen atoms, or when the substituent of R7 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, aryl, heteroaryl, heterocyclyl, aryl C₁₋₆ alkyl, heteroaryl C₁₋₆ alkyl, heterocyclyl C₁₋₆ alkyl, C₁₋₄ alkoxy, R7e, C₁₋₆ alkyl, OH, OR7e, OCOR7e, SH, SR7e, SCOR7e, NH₂, NO₂, NHR7e, NR7eR7f, COR7e, CSR7e, CN, COOH, COOR7e, CONH₂, C(NOH)NH₂, SO₂R7e, SO₃H, SO₂NH₂, CONR7eR7f, SO₂NR7eR7f, wherein R7e and R7f are independently selected from C₁₋₆ alkyl, substituted C₁₋₆ alkyl, aryl, heteroaryl, C₃₋₈ cycloalkyl and heterocyclyl, or R7e and R7f, together with the heteroatom to which they are joined, can form heterocyclyl;
Z can be N, CH or C—R8, wherein R8 is selected from C₁₋₁₀ alkyl, aryl, heteroaryl, heterocyclyl, C₁₋₆ alkoxy, aryloxy, heteroaryloxy, heterocyclyloxy, R8a, halogen, OH, OR8a, SH, SR8a, OCOR8a, SCOR8a, NH_{Z}, NO₂, NHR8a, NR8aR8b, COR8a, CSR5a, CN, COOH, COOR8a, CONH₂, SO₂R8a, SO₃H, SO₂NH₂, CONR8aR8b, SO₂NR8aR8b, wherein R8a and R8b are independently selected from C₁₋₆ alkyl, substituted C₁₋₆ alkyl, aryl, heteroaryl, C₃₋₈ cycloalkyl and heterocyclyl, or R8a and R8b, together with the heteroatom to which they are joined, can form heterocyclyl,
wherein, when R8 is C₁₋₆ alkyl, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R8c, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR8c, OCOR5c, SH, SR8c, SCOR8c, NH₂, NO₂, NHR8c, NR8cR8d, COR8c, CSR8c, CN, COOH, COOR8c, CONH₂, SO₂R8c, SO₃H, SO₂NH₂, CONR8cR8d, SO₂NR8cR8d, wherein R8c and R8d are independently selected from C₁₋₆ alkyl, substituted C₁₋₆ alkyl, aryl, heteroaryl, C₃₋₈ cycloalkyl and heterocyclyl, or R8c and R8d, together with the heteroatom to which they are joined, can form heterocyclyl,
wherein, when the substituent of R8 is C₁₋₆ alkyl, aryl, heteroaryl, heterocyclyl, C₁₋₆ alkoxy, aryloxy, heteroaryloxy, heterocyclyloxy, aryl. C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R8e, C₁₋₆ alkyl, OH, OR8e, OCOR8e, SH, SR8e, SCOR8e, NH₂, NO₂, NHR8e, NR8eR8f, COR8e, CSR8e, CN, COOH, COOR8e, CONH₂, SO₂R8e, SO₃H, SO₂NH₂, CONR8eR8f, SO₂NR8eR8f, wherein R8e and R8f are independently selected from C₁₋₆ alkyl, substituted C₁₋₆ alkyl, aryl, heteroaryl, C₃₋₈ cycloalkyl and heterocyclyl, or R8e and R8f, together with the heteroatom to which they are joined, can form heterocyclyl;
wherein, at most, two of the atoms or groups denoted X, Y and Z can be N;
wherein, when W is N, the CONR1R2 group may be joined to W instead, with the double bonds in Formula I rearranged accordingly;
or a pharmaceutically acceptable salt or ester thereof.

In such an embodiment, the compound may be limited by the following exceptions:

provided that when R1 and R2 together form piperidinyl in compounds having Formula I, the piperidinyl is not substituted with methyl, dimethyl, ethyl, isopropyl, tert-butyl, methoxycarbonyl, trifluoromethyl, chloro, bromo or benzyl,
provided that R1 and R2 together in compounds having Formula I do not form 6,7-dimethoxy-3,4-dihydro-1H-isoquinolin-2-yl, 6-methoxy-3,4-dihydro-1H-isoquinolin-2-yl, 7-methoxy-3,4-dihydro-1H-isoquinolin-2-yl, 7-amino-3,4-dihydro-1H-isoquinolin-2-yl, 7-nitro-3,4-dihydro-1H-isoquinolin-2-yl, 3,4-dihydro-1H-isoquinolin-2-yl, 3,4-dihydro-1H-isoquinolin-1-yl, 3,4-dihydro-2H-quinolin-1-yl, pyrrolidin-1-yl, 3,6-dihydro-2H-pyridin-1-yl, 8-aza-spiro[4.5]dec-8-yl, 1,3-dihydroisoindol-2-yl, octahydroisoindol-2-yl, 1,2,6-triaza-spiro[2.5]oct-1-en-6-yl or azepan-1-yl, and/or
provided that Ring A in compounds having Formula I does not form a pyridine, pyrimidine, substituted pyridine or substituted pyrimidine, when R1 and R2, together with the N to which they are attached, form piperidinyl, piperazinyl, substituted piperidinyl or substituted piperazinyl.

In the preceding embodiments, and in those which follow, it will be appreciated that the process used for preparing the specified groups of compounds of Formula II and Formula I employs an intermediate of Formula II′ or Formula I′ having a corresponding structure in which the —CONR1R2 group of Formula II or Formula I is replaced by H.

Preferably, the compound prepared by the process of the invention has a formula selected from Formula I, Formula IIa, Formula IIb, Formula IIc and Formula IId.

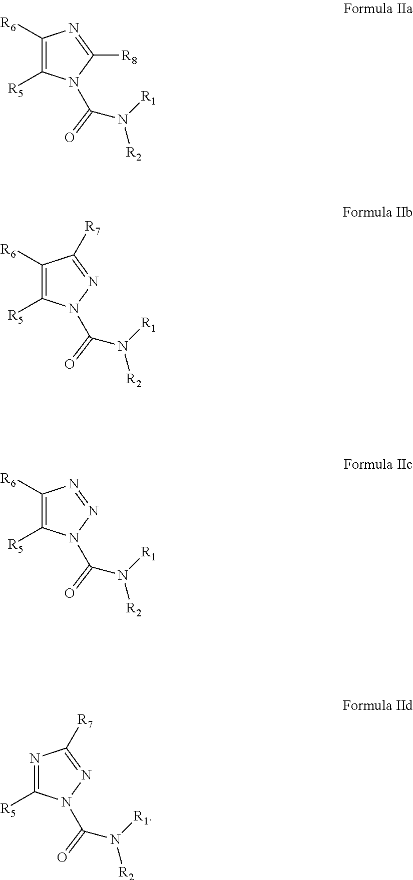

Also preferably, the compound of Formula II or Formula I has a formula selected from Formula Ia, Formula IIa, Formula IIb, Formula IIe and Formula IId.

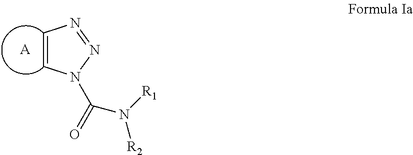

and the intermediate of Formula II′ or Formula I′ has a corresponding structure in which the —CONR1R2 group of Formula IIa-d or Formula Ia is replaced by the H of Formula II′ or Formula I′. In particular embodiments, the compound has the Formula IIa, wherein the intermediate of Formula II′ has a corresponding structure in which the —CONR1R2 group of Formula IIa is replaced by H.

In one embodiment of the invention, R1 is preferably selected from H and C₁₋₄ alkyl. More preferably, R1 is selected from H and C₁₋₃ alkyl, even more preferably, R1 is selected from H, methyl and ethyl and most preferably, R1 is selected from H and methyl.

R2 is preferably selected from C₁₋₄ alkyl, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. Preferably, the aryl, heteroaryl, heterocyclyl and C₃₋₁₀ cycloalkyl (including in aryl C₁₋₆ alkyl, heteroaryl C₁₋₆ alkyl, heterocyclyl C₁₋₆ alkyl and C₃₋₁₀ cycloalkyl C₁₋₆ alkyl) have a 6 membered monocyclic ring structure. More preferably, the aryl, heteroaryl, heterocyclyl and C₃₋₁₀ cycloalkyl (including in aryl C₁₋₆ alkyl, heteroaryl C₁₋₆ alkyl, heterocyclyl C₁₋₆ alkyl and C₃₋₁₀ cycloalkyl C₁₋₆ alkyl) are selected from phenyl, cyclohexyl, phenyl C₁₋₆ alkyl and cyclohexyl C₁₋₆ alkyl, each of which can be substituted or unsubstituted. Preferably, the C₁₋₆ alkyl of each of aryl C₁₋₆ alkyl, heteroaryl C₁₋₆ alkyl, heterocyclyl C₁₋₆ alkyl and C₃₋₁₀ cycloalkyl C₁₋₆ alkyl is a linear alkyl.

Alternatively, R2 can be selected from aryl, heteroaryl, heterocyclyl, aryl C₁₋₆ alkyl, heteroaryl C₁₋₆ alkyl and heterocyclyl C₁₋₆ alkyl, each of which may be substituted or unsubstituted and wherein the aryl, heteroaryl and heterocyclyl (including in aryl C₁₋₆ alkyl, heteroaryl C₁₋₆ alkyl and heterocyclyl C₁₋₆ alkyl) have a bicyclic ring structure, preferably, a 10 membered bicyclic ring structure. More preferably, R2 is selected from naphthalenyl and naphthalenyl C₁₋₆ alkyl.

Each of the aryl, heteroaryl, heterocyclyl and C₃₋₁₀ cycloalkyl groups of R2 (including in aryl C₁₋₆ alkyl, heteroaryl C₁₋₆ alkyl, heterocyclyl C₁₋₆ alkyl and C₃₋₁₀ cycloalkyl C₁₋₆ alkyl) can be substituted with one or more halogens.

Alternatively, each of the aryl, heteroaryl, heterocyclyl and C₃₋₁₀ cycloalkyl groups (including in aryl C₁₋₆ alkyl, heteroaryl C₁₋₆ alkyl, heterocyclyl C₁₋₆ alkyl and C₃₋₁₀ cycloalkyl C₁₋₆ alkyl) can be substituted with C₁₋₄ alkoxy or aryloxy. Preferably, the C₁₋₄ alkoxy is methoxy or ethoxy. Preferably, the aryloxy is monocyclic aryloxy and, more preferably, phenoxy.

In a preferred embodiment, R1 is selected from H and C₁₋₄ alkyl, and R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. More preferably, R1 is selected from H, methyl and ethyl, and R2 is selected from aryl, heteroaryl, heterocyclyl, and C₃₋₁₀ cycloalkyl each of which may be substituted or unsubstituted. More preferably, R1 is methyl. More preferably, R2 is selected from aryl, heteroaryl, heterocyclyl, and C₅₋₈ cycloalkyl each of which are monocyclic and may be substituted or unsubstituted. More preferably still, R2 is selected from saturated heterocyclyl, and C₅₋₈ cycloalkyl each of which are monocyclic and may be substituted or unsubstituted. When R2 is a monocyclic C₅₋₈ cycloalkyl, it is preferably unsubstituted. Preferably, R2 is a cyclohexyl, such as an unsubstituted cyclohexyl. When R2 is a monocyclic saturated heterocyclyl, the heterocyclyl ring preferably contains a single heteroatom. Preferably, the heteroatom is a nitrogen or oxygen atom. More preferably, the heterocyclyl is six membered, such as a piperidinyl or tetrahydropyranyl group. If the heteroatom is an oxygen atom, the heterocyclyl is preferably unsubstituted. If the heteroatom is a nitrogen atom, the nitrogen heteroatom may be substituted or unsubstituted. If the nitrogen heteroatom is substituted, it is preferably substituted with a group selected from C₁₋₆ alkyl, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. More preferably, the nitrogen heteroatom is substituted with a group selected from C₁₋₄ alkyl, aryl C₁₋₄ alkyl, heteroaryl C₁₋₄ alkyl, heterocyclyl C₁₋₄ alkyl and C₅₋₈ cycloalkyl C₁₋₄ alkyl. More preferably, the nitrogen heteroatom is substituted with a group selected from aryl C₁₋₄ alkyl and heteroaryl C₁₋₄ alkyl, wherein the aryl and heteroaryl are monocyclic and, preferably, six membered. Preferably, the nitrogen heteroatom is substituted with a group selected from phenyl C₁₋₂ alkyl and pyridyl C₁₋₂ alkyl. Preferably, the heteroatom in the said heterocyclyl group is at the 4 position relative to the position of attachment of the heterocyclyl group R2 to the urea nitrogen. When R1 and R2 are as defined in this paragraph, the compound preferably has the formula IIa. Preferably, when R1 and R2 are as defined in this paragraph, R6 is a substituted or unsubstituted aryl or heteroaryl and, preferably, a substituted or unsubstituted monocyclic aryl or heteroaryl. The monocyclic aryl or heteroaryl is preferably six membered. In one embodiment, R6 is a substituted or unsubstituted aryl (such as phenyl) and, preferably, unsubstituted. In another embodiment, R6 is a substituted or unsubstituted heteroaryl and, preferably, substituted or unsubstituted pyridyl. In one embodiment, the heteroaryl is substituted with an oxygen atom. For example, the nitrogen heteroatom of pyridyl may be substituted with an oxygen atom so that it is oxidised, i.e. an N-oxide is formed.

It has been found that compounds with the selection of R1 and R2 in the preceding paragraph show relatively high specificity for FAAH. Further, compounds in which R2 is heterocyclyl, such as piperidinyl or tetrahydropyranyl, have been found to be relatively metabolically stable.

In an alternative embodiment, R2 is preferably C₂₋₂₀ alkyl. More preferably, R2 is C₃₋₁₆ alkyl and, more preferably still, R2 is C₄₋₁₂ alkyl. Preferably, the alkyl in a linear alkyl.

In a preferred embodiment, R1 is selected from H and C₁₋₄ alkyl, and R2 is C₂₋₂₀ alkyl.

In various embodiments, when R1 is: H or C₁₋₄ alkyl; H or C₁₋₃ alkyl; H, methyl or ethyl; H or methyl; or methyl, R2 can be selected from C₁₋₆ alkoxy, aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, halogen, OH, OR1a, OCOR1a, SH, SR1a, SCOR1a, NH₂, NHR1a, NHSO₂NH₂, NHSO₂R1a, NR1aCOR1b, NHCOR1a, NR1aR1b, COR1a, CSR1a, CN, COOH, COOR1a, CONH₂, CONHOH, CONHR1a, CONHOR1a, SO₂R1a, SO₃H, SO₂NH₂, CONR1aR1b, SO₂NR1aR1b, wherein R1a and R1b are independently selected from C₁₋₆ alkyl, substituted C₁₋₆ alkyl, aryl, heteroaryl, C₃₋₈ cycloalkyl and heterocyclyl, or R1a and R1b, together with the heteroatom to which they are joined, can form heterocyclyl, wherein R2 can be substituted or unsubstituted.

Alternatively, in other embodiments, when R1 is: H and C₁₋₄ alkyl; H and C₁₋₃ alkyl; H, methyl and ethyl; H and methyl; or methyl, R2 can be selected from aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, wherein R2 can be substituted or unsubstituted.

In a preferred embodiment, R1 and R2, together with the N to which they are attached, form a heterocyclyl group which may be substituted or unsubstituted. Preferably, the heterocyclyl is a 5 or 6 membered monocyclic ring and, more preferably, a 5 membered monocyclic ring. In certain embodiments, the said heterocyclyl contains one or two, preferably 1, additional heteroatoms (i.e. in addition to the N). These additional heteroatoms may be, for example, N, O and/or S. Preferably, the heterocyclyl is oxazolidinyl. Preferably, the oxygen atom in the oxazolidinyl is at the 3 position relative to the urea nitrogen. Preferably, the oxazolidinyl is substituted with one, two or three methyl or ethyl groups. More preferably, the oxazolidinyl is substituted with two methyl or ethyl groups. More preferably still, the oxazolidinyl is substituted with two methyl groups on the same carbon atom. More preferably, the oxazolidinyl is 4,4-dimethyloxazolidin-3-yl. When R1 and R2 are as defined in this paragraph, the compound preferably has the formula Ia or IIa. Preferably, when R1 and R2 are as defined in this paragraph and the compound has the formula IIa, R6 is a substituted or unsubstituted aryl and, more preferably, phenyl. When R1 and R2 are as defined in this paragraph and the compound has the formula Ia, ring A is preferably an unsubstituted or substituted benzo moiety.

Compounds having R1 and R2 as defined in the preceding paragraph have been found to be relatively potent inhibitors of FAAH. They have also been found to have relatively high specificity for FAAH.

In an alternative embodiment, R1 and R2, together with the N to which they are attached, form a heterocyclyl group which may be substituted or unsubstituted. Preferably, the heterocyclyl is a 5 or 6 membered monocyclic ring and, more preferably, a 6 membered monocyclic ring. Preferably, R1 and R2 together form morpholino, piperazinyl oxazolidinyl, pyrrolidinyl or piperidinyl. More preferably, R1 and R2 together form morpholino or piperazinyl.

Preferably, the heterocyclyl of R1 and R2 together is substituted with C₁₋₄ alkyl, aryl, heteroaryl, C₃₋₈ cycloalkyl aryl C₁₋₆ alkyl, heteroaryl C₁₋₆ alkyl, aryloxy, heteroaryloxy, aryl C₁₋₆ alkoxy and heteroaryl C₁₋₆ alkoxy, each of which may optionally be substituted with one or more halogens or C₁₋₄ alkyl groups. Preferably, the substituent aryl, heteroaryl or C₃₋₈ cycloalkyl is a 5 or 6 membered monocyclic ring. More preferably, the heterocyclyl of R1 and R2 together is substituted with aryl, aryl C₁₋₆ alkyl and aryloxy, each of which may optionally be substituted with one or more halogen. More preferably still, the heterocyclyl of R1 and R2 together is substituted with phenyl, phenyl C₁₋₆ alkyl or phenoxy, each of which may optionally be substituted with one or more halogen.

Alternatively, the heterocyclyl of R1 and R2 together may be substituted with a heteroaryl or heteroaryl C₁₋₆ alkyl. In one embodiment, the heteroaryl has a bicyclic ring structure, for example, benzodioxolylmethyl. Alternatively, the heteroaryl may be monocyclic, for example, pyridyl.

In another alternative, the heterocyclyl of R1 and R2 together may be substituted with a C₃₋₈ cycloalkyl. Preferably, the C₃₋₈ cycloalkyl is a monocyclic cycloalkyl such as cyclohexyl.

In one embodiment, the heterocyclyl of R1 and R2 together can be 1,4-dioxa-8-azaspiro[4.5]dec-8-yl, dimethyloxazolidinyl, methylpiperazinyl, benzyloxyphenylpiperazinyl, tolyloxypiperidinyl, pyrrolidinyl C₁₋₄ alkyl piperidinyl, pyridylpiperidinyl, pyridyloxadiazol-5-ylpiperidinyl or benzyloxypiperidinyl.

In one embodiment, the heterocyclyl of R1 and R2 together is piperidinyl substituted with phenoxy or phenyl C₁₋₄ alkoxy and wherein the phenyl may optionally be substituted with halogen.

In one embodiment of the invention, when V is C—R3, R3 is H or halogen.

In another embodiment of the invention, when W is C—R4, R4 is selected from H and aryl. Preferably, R4 is selected from H and phenyl. More preferably, R4 is H.

In the compound prepared according to the invention, ring A is preferably a substituted or unsubstituted monocyclic aryl or heteroaryl moiety and, more preferably, a monocyclic aryl moiety. Preferably, ring A is a substituted or unsubstituted benzo moiety. When the monocyclic aryl of ring A is substituted, the substituent is one or more of halogen, C₁₋₆ alkyl or aryl which can optionally be substituted with one or more of halogen, cyano, carboxylic acid or amide. Preferably, the substituent aryl is monocyclic aryl and, more preferably, phenyl. In a preferred embodiment, the compound, having ring A as defined in this paragraph, has formula Ia.

In one embodiment, ring A is substituted with a moiety selected from C₁₋₆ alkoxy, C₁₋₆ alkoxy C₁₋₆ alkyl, and C₀₋₆ alkyl-CO—C₀₋₆ alkyl, wherein the C₁₋₆ alkoxy, C₁₋₆ alkoxy C₁₋₆ alkyl, or C₀₋₆ alkyl-CO—C₀₋₆ alkyl is substituted with a moiety selected from aryl, heteroaryl, heterocyclyl, and C₃₋₁₀ cycloalkyl, wherein each of these moieties may optionally be substituted with aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl, and C₃₋₁₀ cycloalkyl C₁₋₆ alkyl. Preferably, ring A is substituted with a C₀₋₆ alkyl-CO—C₀₋₆ alkyl, wherein the C₀₋₆ alkyl-CO—C₀₋₆ alkyl is substituted with a moiety selected from aryl, heteroaryl, heterocyclyl, and C₃₋₁₀ cycloalkyl, wherein each of these moieties may optionally be substituted with aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl, and C₃₋₁₀ cycloalkyl C₁₋₆ alkyl. Preferably, ring A is substituted with a carbonyl moiety (i.e. C₀ alkyl-CO—C₀ alkyl). Preferably, the C₀₋₆ alkyl-CO—C₀₋₆ alkyl is substituted with a heterocyclyl, more preferably, a monocyclic heterocyclyl, more preferably still, a heterocyclyl containing one or two nitrogen heteroatoms, even more preferably, a six membered heterocyclyl, and most preferably, piperazine. Preferably, the C₁₋₆ alkoxy, C₁₋₆ alkoxy C₁₋₆ alkyl, or C₀₋₆ alkyl-CO—C₀₋₆ alkyl is linear. Preferably, compounds as described in this paragraph are of formula Ia.

In another embodiment, ring A is substituted with one or more groups selected from halogen, C₁₋₆ alkyl, C₁₋₆ alkoxy, 01-1, ORa, OCORa, SH, SRa, SCORa, NH₂, NO₂, NHRa, NHSO₂NH₂, NHSO₂Ra, NRaCORb, NHCORa, NHC(NH)NH₂, NRaRb, CORa, CSRa, CN, COOH, COORa, CONH₂, CONHRa, CONHOH, CONHORa, C(NOH)NH₂, CONRaRb, SO₂Ra, SO₃H, SO₂NH₂, SO₂NRaRb, wherein Ra and Rb are C₁₋₆ alkyl. Preferably, ring A is substituted with one or more groups selected from halogen, OH, SH, NH₂, NO₂, NHC(NH)NH₂, CN, COOH, CONH₂, CONHOH, C(NOH)NH₂, SO₃H, and SO₂NH₂. More preferably, ring A is substituted with one or more groups selected from halogen, OH, NH₂, NO₂, NHC(NH)NH₂, CN, COOH, CONH₂, CONHOH, C(NOH)NH₂, SO₃H, and SO₂NH₂. Preferably, compounds as described in this paragraph are of formula Ia.

Preferably, in the compound prepared according to the invention, R5 is H or halogen, and, more preferably, R5 is H.

In one embodiment, R5 together with the ring carbon to which it is attached, does not form a carbonyl group. The compound is of Formula II as indicated above.

In another embodiment, X is not O. The compound is of Formula II as indicated above.

In compounds having Formula II, when X is C—R6, R6 is preferably a substituted or unsubstituted aryl or a substituted or unsubstituted heroaryl. Preferably, the aryl R6 is phenyl or naphthalenyl. More preferably, the aryl R6 is phenyl. Preferably, the aryl R6 is substituted with one or more groups selected from halogen, C₁₋₄ alkoxy, hydroxyl, amide, nitro, aryl, heterocyclyl, heteroaryl, heterocyclyl, aryloxy, each of which may be substituted or unsubstituted. Preferably, the aryl substituent of R6 is phenyl which may be substituted or unsubstituted. When R6 is defined as in this paragraph, the compound of Formula II is preferably an imidazole (i.e. X is CH or C—R6, Y is N, and Z is CH or C—R8) or a 1,2,3-triazole (i.e. X is CH or C—R6, Y is N, and Z is N). More preferably, the compound has formula IIa.

Alternatively, R6 is preferably H, halogen or aryl and, more preferably, H. When R6 is defined as in this paragraph, the compound of Formula II is preferably a pyrazole (i.e. X is CH or C—R6, Y is CH or C—R7, and Z is N).

In one embodiment of the invention, when Y is C—R7, R7 is selected from aryl or heteroaryl, each of which can be substituted or unsubstituted. Preferably, the aryl and heteroaryl are monocyclic. Preferably, the aryl or heteroaryl is substituted with one or more halogens. In a preferred embodiment of the invention, R7 is substituted or unsubstituted aryl. When R7 is defined as in this paragraph, the compound of Formula II is preferably a pyrazole (i.e. X is CH or C—R6, Y is CH or C—R7, and Z is N) or a 1,2,4-triazole (i.e. X is N, Y is CH or C—R7, and Z is N).

In one embodiment, when Y is C—R7, R7 is H.

In another embodiment of the invention, when Z is C—R8, R8 is selected from H and aryl. Preferably, R8 is selected from H and phenyl. More preferably, R8 is H.

In one embodiment of the invention, R6 is a group selected from aryl, heteroaryl, heterocyclyl, C₃₋₁₀ cycloalkyl, wherein the R6 group is substituted with a group selected from C₁₋₆ alkoxy, C₁₋₆ alkoxy C₁₋₆ alkyl, and C₀₋₆ alkyl-CO—C₀₋₆ alkyl, wherein the C₁₋₆ alkoxy, C₁₋₆ alkoxy C₁₋₆ alkyl, or C₀₋₆ alkyl-CO—C₀₋₆ alkyl group is substituted with a group selected from aryl, heteroaryl, heterocyclyl, and C₃₋₁₀ cycloalkyl. Preferably, R6 is a group selected from aryl, heteroaryl, heterocyclyl, C₃₋₁₀ cycloalkyl, wherein the R6 group is substituted with a group selected from C₁₋₆ alkoxy and C₁₋₆ alkoxy C₁₋₆ alkyl, wherein the C₁₋₆ alkoxy or C₁₋₆ alkoxy C₁₋₆ alkyl group is substituted with a group selected from aryl, heteroaryl, heterocyclyl, and C₃₋₁₀ cycloalkyl. Preferably, R6 is a group selected from aryl, heteroaryl, heterocyclyl, C₃₋₁₀ cycloalkyl, wherein the R6 group is substituted with a group selected from C₁₋₆ alkoxy and C₁₋₆ alkoxy C₁₋₆ alkyl, wherein the C₁₋₆ alkoxy or C₁₋₆ alkoxy C₁₋₆ alkyl group is substituted with a heterocyclyl. More preferably, R6 is an aryl which is substituted with a group selected from C₁₋₆ alkoxy and C₁₋₆ alkoxy C₁₋₆ alkyl, wherein the C₁₋₆ alkoxy or C₁₋₆ alkoxy C₁₋₆ alkyl group is substituted with a heterocyclyl. More preferably still, R6 is an aryl which is substituted with C₁₋₆ alkoxy, wherein the C₁₋₆ alkoxy is substituted with a heterocyclyl.

Preferably, R6 is an aryl or heteroaryl. Preferably, R6 has a monocyclic ring structure such as a monocyclic aryl or heteroaryl. In one embodiment, R6 has a six membered ring structure such as phenyl or pyridyl.

Preferably, the C₁₋₆ alkoxy, C₁₋₆ alkoxy C₁₋₆ alkyl or C₀₋₆ alkyl-CO—C₀₋₆ alkyl is linear.

Preferably, the substituent of the C₁₋₆ alkoxy or C₁₋₆ alkoxy C₁₋₆ alkyl is monocyclic. Preferably, the substituent of the C₁₋₆ alkoxy or C₁₋₆ alkoxy C₁₋₆ alkyl is six membered. Preferably, the substituent of the C₁₋₆ alkoxy or C₁₋₆ alkoxy C₁₋₆ alkyl is heterocyclyl. Preferably, the heterocyclyl is fully saturated. Preferably, the heterocyclyl contains one or two heteroatoms such as nitrogen or oxygen. Preferably, the heterocyclyl contains at least one nitrogen heteroatom. In one embodiment, the heterocyclyl is piperidinyl, piperazinyl, or tetrahydropyranyl. In this embodiment, the compound preferably is of formula IIa.

In one embodiment, when W is N, the CONR1R2 group may not be joined to W instead. In this embodiment, the compound is of Formula I as indicated above.

### Formula I and Ia

In compounds having formula I and, in particular, compounds having formula Ia, ring A is preferably a substituted or unsubstituted aryl or heteroaryl moiety. More preferably, ring A is a substituted or unsubstituted monocyclic aryl or heteroaryl moiety. More preferably still, ring A is a substituted or unsubstituted six-membered aryl or heteroaryl moiety. Most preferably, ring A is a substituted or unsubstituted monocyclic aryl such as a benzo moiety.

When ring A is substituted, the substituent may be one or more groups selected from halogen, OH, C₁₋₄ alkyl, C₁₋₄ alkoxy, SH, NH₂, NO₂, CN, COOH, CONH₂, CONHOH, benzoxyaminocarbonyl, SO₃H, SO₂NH₂, aryl, heteroaryl, heterocyclyl, and C₃₋₈ cycloalkyl. When the substituent is C₁₋₄ alkyl, aryl, heteroaryl, heterocyclyl, or C₃₋₈ cycloalkyl, each of these moieties may optionally be substituted with one or more groups selected from halogen, OH, SH, NH₂, NO₂, CN, COOH, CONH₂, SO₃H, SO₂NH₂, C₁₋₃ alkyl, C₁₋₃ alkoxy and benzyl.

Preferably, the substituent of ring A is one or more groups selected from halogen, OH, C₁₋₃ alkyl, C₁₋₃ alkoxy, NH₂, NO₂, CN, COOH, CONH₂, monocyclic aryl, monocyclic heteroaryl, monocyclic heterocyclyl, and C₅₋₈ cycloalkyl. When the substituent is C₁₋₃ alkyl, monocyclic aryl, monocyclic heteroaryl, monocyclic heterocyclyl or C₅₋₈ cycloalkyl, each of these moieties may optionally be substituted with one or more groups selected from halogen, CN, COOH, CONH₂, and C₁₋₃ alkoxy.

More preferably, the substituent of ring A is one or more groups selected from halogen, OH, C₁₋₂ alkyl, C₁₋₂ alkoxy, and phenyl. When the substituent is C₁₋₂ alkyl or phenyl, each of these moieties may optionally be substituted with one or more groups selected from halogen, CN, COOH, CONH₂, and C₁₋₃ alkoxy.

In a preferred embodiment of compounds having formula I and, in particular, compounds having formula Ia, R1 and R2, together with the N to which they are attached, form a heterocyclyl group which may be substituted or unsubstituted. Preferably, the heterocyclyl is a 5 or 6 membered monocyclic ring, more preferably, a 6 membered monocyclic ring. In certain embodiments, the said heterocyclyl contains one or two, preferably 1, additional heteroatoms (i.e. in addition to the N). These additional heteroatoms may be, for example, N, O and/or S. In one embodiment, the heterocyclyl is morpholino. In an alternative embodiment, the heterocyclyl is piperazinyl. In other embodiments, the said heterocyclyl contains no additional heteroatoms (i.e. it contains a single N atom). In one embodiment, the heterocyclyl is piperidinyl. Where the heterocyclyl is substituted, it is preferably substituted with an aryl or an aryl C₁₋₄ alkyl, wherein the aryl is preferably monocyclic and more preferably phenyl. The alkyl is preferably linear. More preferably, the heterocyclyl is substituted with an aryl or an aryl C₁₋₂ alkyl, wherein the aryl is preferably monocyclic and more preferably phenyl.

In a preferred embodiment of compounds having formula I and, in particular, compounds having formula Ia, R1 is selected from H and C₁₋₄ alkyl, and R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. In one embodiment, R1 is selected from H, methyl and ethyl, and R2 is selected from aryl, heteroaryl, heterocyclyl, and C₅₋₈ cycloalkyl, each of which are monocyclic and may be substituted or unsubstituted. More preferably, R1 is selected from H and methyl. In one embodiment, R1 is methyl. In an alternative embodiment, R1 is H. More preferably, R2 is selected from saturated heterocyclyl, and C₅₋₈ cycloalkyl, each of which are monocyclic and may be substituted or unsubstituted. When R2 is a monocyclic C₅₋₈ cycloalkyl, it is preferably unsubstituted. Preferably, R2 is a cyclopentyl or cyclohexyl. More preferably, R2 is a cyclohexyl, such as an unsubstituted cyclohexyl. When R2 is a monocyclic saturated heterocyclyl, the heterocyclyl ring preferably contains a single heteroatom. More preferably, the heterocyclyl is six membered, such as a piperidinyl or tetrahydropyranyl group. The nitrogen heteroatom may be substituted or unsubstituted.

In an alternative embodiment, R1 is selected from H, methyl and ethyl, and R2 is selected from aryl C₁₋₆ alkyl, heteroaryl C₁₋₆ alkyl, heterocyclyl C₁₋₆ alkyl, and C₅₋₈ cycloalkyl C₁₋₆ alkyl, each of which are monocyclic and may be substituted or unsubstituted. More preferably, R2 is aryl C₁₋₆ alkyl in which the aryl is monocyclic and may be substituted or unsubstituted. More preferably still, R2 is aryl C₁₋₆ alkyl in which the aryl is monocyclic and may be substituted or unsubstituted and the C₁₋₆ alkyl is linear. Even more preferably, R2 is phenyl C₁₋₆ alkyl which may be substituted or unsubstituted and the C₁₋₆ alkyl is linear. In one embodiment, the phenyl is unsubstituted.

In an alternative embodiment, R1 is selected from H, methyl and ethyl, and R2 is C₁₋₄ alkyl substituted with a group selected from aryl C₁₋₄ alkoxy, heteroaryl C₁₋₄ alkoxy, heterocyclyl C₁₋₄ alkoxy, and C₅₋₈ cycloalkyl C₁₋₄ alkoxy, each of which are monocyclic and may be substituted or unsubstituted. Preferably, R2 is substituted C₁₋₃ alkyl. In one embodiment, R2 is substituted C₁₋₂ alkyl. Preferably, the substituent of R2 is aryl C₁₋₄ alkoxy in which the aryl is monocyclic and may be substituted or unsubstituted. More preferably still, the substituent of R2 is aryl C₁₋₄ alkoxy in which the aryl is monocyclic and may be substituted or unsubstituted and the C₁₋₄ alkoxy is linear. Even more preferably, the substituent of R2 is phenyl C₁₋₄ alkoxy which may be substituted or unsubstituted and the C₁₋₄ alkoxy is linear. In one embodiment, the substituent of R2 is aryl C₁₋₃ alkoxy in which the aryl is monocyclic (e.g. phenyl) and may be substituted or unsubstituted and the C₁₋₃ alkoxy is linear. In some embodiments, the phenyl is unsubstituted.

In yet another embodiment of compounds having formula I and, in particular, compounds having formula Ia, R1 is selected from H and C₁₋₄ alkyl, and R2 is selected from heterocyclyl which may be substituted or unsubstituted. Preferably, R1 is H, methyl or ethyl, and R2 is a bicyclic heterocyclyl which may be substituted or unsubstituted. More preferably, R1 is H or methyl, and R2 is a bicyclic heterocyclyl which may be substituted or unsubstituted, wherein one of the rings of the heterocyclyl contains two oxygen atoms. In one embodiment, R2 is 3,3-dimethyl-1,5-dioxaspiro[5.5]undec-9-yl.

In an alternative preferred embodiment of compounds having formula I and, in particular, compounds having formula Ia, R1 is selected from H and C₁₋₄ alkyl, and R2 is C₂₋₂₀ alkyl. More preferably, R1 is H, methyl or ethyl and more preferably still, R1 is H or methyl. Preferably, R2 is C₃₋₁₆ alkyl, wherein the alkyl is a linear alkyl. More preferably, R2 is C₄₋₁₄ alkyl, wherein the alkyl is a linear alkyl.

### Formula IIa

In a preferred embodiment of compounds having Formula IIa, R1 is selected from H and C₁₋₄ alkyl, and R2 is selected from C₁₋₆ alkyl, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. More preferably, R1 is selected from H, methyl and ethyl, and R2 is selected from aryl, heteroaryl, heterocyclyl, and C₃₋₁₀ cycloalkyl each of which may be substituted or unsubstituted. More preferably, R1 is methyl. More preferably, R2 is selected from aryl, heteroaryl, heterocyclyl, and C₅₋₈ cycloalkyl each of which are monocyclic and may be substituted or unsubstituted. More preferably still, R2 is selected from aryl such as phenyl, saturated heterocyclyl, and C₅₋₈ cycloalkyl each of which are monocyclic and may be substituted or unsubstituted. When R2 is a monocyclic C₅₋₈ cycloalkyl (i.e. cyclopentyl, cyclohexyl, cycloheptyl or cyclooctyl) or aryl, it is preferably unsubstituted. Preferably, R2 is a cyclohexyl, such as an unsubstituted cyclohexyl. When R2 is a monocyclic saturated heterocyclyl, the heterocyclyl ring preferably contains a single heteroatom, such as nitrogen or oxygen. More preferably, the heterocyclyl is six membered, such as a piperidinyl or tetrahydropyranyl group. In one embodiment, the heteroatom is a nitrogen heteroatom which may be substituted or unsubstituted. Preferably, the heteroatom in the said heterocyclyl group is at the 4-position relative to the position of attachment of the heterocyclyl group R2 to the urea nitrogen. In one embodiment, the nitrogen atom is substituted with monocyclic aryl (preferably phenyl) C₁₋₃ alkyl; preferably, the nitrogen atom is substituted with benzyl or phenylethyl; and, more preferably, the nitrogen atom is substituted with benzyl.

In an alternative preferred embodiment of compounds having Formula IIa, R1 and R2, together with the N to which they are attached, form a heterocyclyl group which may be substituted or unsubstituted. Preferably, the heterocyclyl is a 5 or 6 membered monocyclic ring and, more preferably, a 5 membered monocyclic ring. In certain embodiments, the said heterocyclyl contains one or two, preferably 1, additional heteroatoms (i.e. in addition to the N). These additional heteroatoms may be, for example, N, O and/or S. Preferably, the heterocyclyl is oxazolidinyl. Preferably, the oxygen atom in the oxazolidinyl is at the 3 position relative to the urea nitrogen. Preferably, the oxazolidinyl is substituted with one, two or three methyl or ethyl groups. More preferably, the oxazolidinyl is substituted with two methyl or ethyl groups. More preferably still, the oxazolidinyl is substituted with two methyl groups on the same carbon atom. More preferably, the oxazolidinyl is 4,4-dimethyloxazolidin-3-yl.

In yet another preferred embodiment of compounds having formula IIa, R1 and R2, together with the N to which they are attached, form a heterocyclyl group which may be substituted or unsubstituted. Preferably, the heterocyclyl is a 5 or 6 membered monocyclic ring, more preferably, a 6 membered monocyclic ring. In certain embodiments, the said heterocyclyl contains one or two, preferably 1, additional heteroatoms (i.e. in addition to the N). These additional heteroatoms may be, for example, N, O and/or S. In one embodiment, the heterocyclyl is morpholino. In an alternative embodiment, the heterocyclyl is piperazinyl. In other embodiments, the said heterocyclyl contains no additional heteroatoms (i.e. it contains a single N atom). In one embodiment, the heterocyclyl is piperadinyl. Where the heterocyclyl is substituted, it is preferably substituted with aryl, aryl C₁₋₄ alkyl, C₅₋₆ cycloalkyl, or C₅₋₆ cycloalkyl C₁₋₄ alkyl, wherein the aryl is preferably monocyclic and more preferably phenyl, and the cycloalkyl is preferably cyclohexyl. The alkyl is preferably linear. In one embodiment, the heterocyclyl is substituted with an aryl or an aryl C₁₋₄ alkyl (preferably C₁₋₂ alkyl), wherein the aryl is preferably monocyclic and more preferably phenyl. The aryl may optionally be substituted with one or more halogen atoms.

In compounds having formula IIa, R5 is preferably selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NO₂, NHR5a, NR5aR5b, COR5a, CSR5a, CN, COOH, COOR5a, CONH₂, SO₂R5a, SO₃H, SO₂NH₂, CONR5aR5b, SO₂NR5aR5b, wherein R5a and R5b are independently selected from C₁₋₆ alkyl, aryl, heteroaryl, C₃₋₈ cycloalkyl and heterocyclyl, and R5a and R5b, together with the heteroatom to which they are joined, can form heterocyclyl. More preferably, R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, halogen, OH, SH, NH₂, NO₂, CN, COOH, CONH₂, SO₃H, SO₂NH₂. More preferably still, R5 is selected from H, C₁₋₄ alkyl, aryl, heteroaryl, heterocyclyl, C₅₋₈ cycloalkyl, C₁₋₄ alkoxy, aryloxy, heteroaryloxy, heterocyclyloxy, halogen, OH, SH, NH₂, NO₂, CN, COOH, CONH₂, SO₃H, SO₂NH₂, wherein the aryl, heteroaryl, heterocyclyl and C₅₋₈ cycloalkyl groups are monocyclic. Even more preferably, R5 is selected from H, C₁₋₃ alkyl, aryl, heteroaryl, heterocyclyl, C₅₋₈ cycloalkyl, halogen, OH, NH₂, COOH and CONH₂, wherein the aryl, heteroaryl, heterocyclyl and C₅₋₈ cycloalkyl groups are monocyclic. More preferably still, R5 is selected from H, C₁₋₂ alkyl and halogen. Even more preferably, R5 is selected from H and halogen such as F, Cl and Br. In one embodiment, R5 is H.

In compounds having formula IIa, R6 is preferably selected from aryl, heteroaryl, heterocyclyl and C₃₋₈ cycloalkyl, each of which may be substituted or unsubstituted. More preferably, R6 is selected from aryl and heteroaryl each of which may be substituted or unsubstituted. In one embodiment, the heteroaryl contains one heteroatom, e.g. an oxygen or nitrogen atom. Preferably, the aryl or heteroaryl is monocyclic. More preferably, the aryl or heteroaryl is a six membered monocyclic ring, for example, phenyl or pyridyl. In one embodiment, the heteroaryl contains a nitrogen atom which is substituted with an oxygen atom such as oxidopyridyl. In another embodiment, R6 is unsubstituted monocyclic aryl such as phenyl, or monocyclic aryl such as phenyl substituted with one or more groups selected from halogen, C₁₋₂ alkoxy (optionally substituted with one or more halogen atoms), or OH.

In one embodiment, R6 is unsubstituted or substituted 2-oxo-2,3-dihydro-1H-benzo[d]imidazolyl.

When R6 is substituted, the substituent is preferably one or more groups selected from halogen, C₁₋₄ alkoxy, aryl, heteroaryl, heterocyclyl, OH, CN, CONH₂, NH₂, heterocyclyl C₁₋₄ alkoxy, aryl C₁₋₄ alkoxy, heteroaryl C₁₋₄ alkoxy, NO₂, SO₂NH₂, SO₃, C(NOH)NH₂, CONHOH, 2H-tetrazol-5-yl, dimethylamino, benzylamino, methylsulfonyl, morpholinosulfonyl and piperidinylsulfonyl. The piperidinylsulfonyl may optionally be substituted with arylmethoxy (preferably benzoxy) or OH. Preferably, the aryl, heteroaryl and heterocyclyl are monocyclic. In one embodiment, the aryl, heteroaryl and heterocyclyl are six-membered monocyclic rings. In a particular embodiment in which R6 is monocyclic aryl, it may optionally be substituted with one or more groups selected from halogen, OH, C₁₋₃ alkoxy (preferably C₁₋₂ alkoxy), aryl (e.g. a monocyclic aryl such as phenyl), heteroaryl (e.g. monocyclic heteroaryl containing one or two nitrogen atoms, or one oxygen atom), heterocyclyl (e.g. piperazinyl, piperadinyl or morpholino) C₁₋₃ alkoxy (preferably C₁₋₂ alkoxy), aryl (e.g. monocyclic aryl such as phenyl) C₁₋₃ alkoxy (preferably C₁₋₂ alkoxy), CONH₂, NH₂, NO₂, OCHF₂, SO₂NH₂, morpholinosulfonyl and C(NOH)NH₂.

In another embodiment in which R6 is monocyclic aryl, it may optionally be substituted with one or more groups selected from halogen, OH, methoxy, phenyl, pyridyl, pyrazinyl, pyranyl, piperazinylmethoxy, piperadinylmethoxy, morpholinomethoxy, benzyloxy, CONH₂, NH₂, NO₂, OCHF₂, SO₂NH₂, rnorpholinosulfonyl and C(NOH)NH₂.

In one embodiment when R6 is monocyclic aryl such as phenyl, the substituent of R6 is aryl, preferably monocyclic aryl such as phenyl, which may be substituted or unsubstituted. Where it is substituted, preferably it is substituted with CONH₂.

When the substituent of R6 is C₁₋₄ alkoxy, aryl, heteroaryl, heterocyclyl, heterocyclyl C₁₋₄ alkoxy, aryl C₁₋₄ alkoxy, heteroaryl C₁₋₄ alkoxy or SO₃, each of these moieties may optionally be substituted with one or more groups selected from halogen, OH, C₁₋₃ alkoxy (which may be substituted with one or more halogen), CONH₂, CN, NCH₃CH₃, NHCOCH₃, methylhydroxybutyl, and methylhydroxybutynyl.

In compounds having formula IIa, R8 is preferably selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R8a, halogen, OH, OR8a, SH, SR8a, OCOR8a, SCOR8a, NH₂, NO₂, NHR5a, NR5aR8b, COR5a, CSR8a, CN, COOH, COOR8a, CONH₂, SO₂R8a, SO₃H, SO₂NH₂, CONR8aR8b, SO₂NR8aR8b, wherein R8a and R8b are independently selected from C₁₋₆ alkyl, aryl, heteroaryl, C₃₋₈ cycloalkyl and heterocyclyl, and R8a and R8b, together with the heteroatom to which they are joined, can form heterocyclyl. More preferably, R8 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, halogen, OH, SH, NH₂, NO₂, CN, COOH, CONH₂, SO₃H, SO₂NH₂. More preferably still, R8 is selected from H, C₁₋₄ alkyl, aryl, heteroaryl, heterocyclyl, C₅₋₈ cycloalkyl, C₁₋₄ alkoxy, aryloxy, heteroaryloxy, heterocyclyloxy, halogen, OH, SH, NH₂, NO₂, CN, COOH, CONH₂, SO₃H, SO₂NH₂, wherein the aryl, heteroaryl, heterocyclyl and C₅₋₈ cycloalkyl groups are monocyclic. Even more preferably, R8 is selected from H, C₁₋₃ alkyl, aryl, heteroaryl, heterocyclyl, C₃₋₈ cycloalkyl, halogen, OH, NH₂, COOH and CONH₂, wherein the aryl, heteroaryl, heterocyclyl and C₅₋₈ cycloalkyl groups are monocyclic. More preferably still, R8 is selected from H, C₁₋₂ alkyl, halogen and monocyclic aryl such as phenyl. Even more preferably, R8 is selected from H, C₁₋₂ alkyl, and halogen such as F, Cl and Br. More preferably still, R8 is selected from H and halogen such as F, Cl and Br. In one embodiment, R8 is H.

In one embodiment of compounds having formula IIa, R1 is selected from H and C₁₋₄ alkyl,

R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may optionally be substituted with one or more groups selected from R2a, halogen, OH, OR2a, OCOR2a, SH, SR2a, SCOR2a, NH₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, SO₂R2a, SO₃H, SO₂NH₂, CONR2aR2b, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of R2 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R2c, halogen, OH, OR2c, OCOR2c, SH, SR2c, SCOR2c, NH₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, SO₂R2c, SO₃H, SO₂NH₂, CONR2cR2d, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
R5 is selected from H, R5a, halogen, OH, OR5a, OCOR5a, SH, SR5a, SCOR5a, NH₂, NHR5a, NHSO₂NH₂, NHSO₂R5a, NR5aCOR5b, NHC(NH)NH₂, NHCOR5a, NR5aR5b, COR5a, CSR5a, CN, COOH, COOR5a, CONH₂, CONHOH, CONHR5a, CONHOR5a, C(NOH)NH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl,
R6 is selected from aryl, heteroaryl, heterocyclyl, C₃₋₁₀ cycloalkyl, each of which may optionally be substituted with one or more groups selected from R6a, halogen, OH, OR6a, OCOR6a, SH, SR6a, SCOR6a, NO₂, NH₂, NHR6a, NHSO₂NH₂, NHSO₂R6a, NR6aCOR6b, NHC(NH)NH₂, NHCOR6a, NR6aR6b, COR6a, CSR6a, CN, COOH, COOR6a, CONH₂, CONHOH, CONHR6a, CONHOR6a, C(NOH)NH₂, SO₂R6a, SO₃H, SO₂NH₂, CONR6aR6b, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl, and wherein, when R6 is heteroaryl or heterocyclyl, each of these moieties may optionally be substituted with one or more oxygen atoms,
wherein, when the substituent of R6 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R6c, halogen, OH, OR6c, OCOR6c, SH, SR6c, SCOR6c, NH₂, NHR6c, NHSO₂NH₂, NHSO₂R6c, NR6cCOR6d, NHC(NH)NH₂, NHCOR6c, NR6cR6d, COR6c, CSR6c, CN, COOH, COOR6c, CONH₂, CONHOH, CONHR6c, CONHOR6c, C(NOH)NH₂, SO₂R6c, SO₃H, SO₂NH₂, CONR6cR6d, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl, and wherein, when the substituent of R6 is heteroaryl or heterocyclyl, each of these moieties may optionally be substituted with one or more oxygen atoms, and
R8 is selected from H, R8a, halogen, OH, OR8a, OCOR8a, SH, SR8a, SCOR8a, NH₂, NHR8a, NHSO₂NH₂, NHSO₂R8a, NR8aCOR8b, NHC(NH)NH₂, NHCOR8a, NR8aR8b, COR8a, CSR8a, CN, COOH, COOR8a, CONH₂, CONHOH, CONHR8a, CONHOR8a, C(NOH)NH₂, SO₂R8a, SO₃H, SO₂NH₂, CONR8aR8b, SO₂NR8aR8b, wherein R8a and R8b are independently selected from C₁₋₆ alkyl, substituted C₁₋₆ alkyl, aryl, heteroaryl, C₃₋₈ cycloalkyl and heterocyclyl, or R8a and R8b, together with the heteroatom to which they are joined, can form heterocyclyl.

In the above embodiment, preferably, R1 is selected from H, methyl and ethyl, and R2 is selected from aryl, heteroaryl, heterocyclyl, and C₃₋₁₀ cycloalkyl each of which may be substituted or unsubstituted. More preferably, R1 is methyl. More preferably, R2 is selected from aryl, heteroaryl, heterocyclyl, and C₅₋₈ cycloalkyl each of which are monocyclic and may be substituted or unsubstituted. More preferably still, R2 is selected from heterocyclyl, and C₅₋₈ cycloalkyl each of which are monocyclic and may be substituted or unsubstituted. Preferably, the heterocyclyl is fully saturated. When R2 is a monocyclic C₅₋₈ cycloalkyl (i.e. cyclopentyl, cyclohexyl, cycloheptyl or cyclooctyl), it is preferably unsubstituted. In one embodiment, R2 is a cyclopentyl or a cyclohexyl, such as an unsubstituted cyclopentyl or unsubstituted cyclohexyl. When R2 is a monocyclic saturated heterocyclyl, the heterocyclyl ring preferably contains a single heteroatom, such as nitrogen or oxygen. Preferably, the heterocyclyl is six membered, such as a piperidinyl or tetrahydropyranyl group. Preferably, the heteroatom in the said heterocyclyl group is at the 4-position relative to the position of attachment of the heterocyclyl group R2 to the urea nitrogen. In one embodiment, the heteroatom is a nitrogen heteroatom which may be substituted or unsubstituted.

In a particular embodiment, the nitrogen atom is substituted with a group selected from CN, CONH₂, C(NOH)NH₂, SO₂—C₁₋₄ alkyl, SO₂-aryl (optionally substituted with a C₁₋₄ alkyl or C₁₋₄ haloalkyl, such as trifluoromethyl), CO-heteroaryl (optionally substituted with a heteroaryl or halogen), CO—C₁₋₄ alkyl, COO—C₁₋₄ alkyl, C₁₋₄ alkyl (optionally substituted with OH, CN, COOH), aryl C₁₋₃ alkyl, heteroaryl C₁₋₃ alkyl such as piperidinyl C₁₋₃ alkyl (optionally substituted with COO—C₁₋₃ alkyl), heterocyclyl C₁₋₃ alkyl, aryl, heteroaryl (optionally substituted with one or more halogens such as chlorine), and heterocyclyl. Preferably, the nitrogen atom is substituted with a group selected from CN, CONH₂, C(NOH)NH₂, SO₂—C₁₋₄ alkyl, SO₂-monocyclic aryl (optionally substituted with a C₁₋₄ haloalkyl, such as trifluoromethyl), CO-monocyclic heteroaryl (optionally substituted with a monocyclic heteroaryl or halogen), CO—C₁₋₄ alkyl, COO—C₁₋₄ alkyl, C₁₋₄ alkyl (optionally substituted with OH, CN, COOH), monocyclic aryl C₁₋₃ alkyl, monocyclic heteroaryl C₁₋₃ alkyl such as piperidinyl C₁₋₃ alkyl (optionally substituted with COO— C₁₋₃ alkyl), monocyclic heterocyclyl C₁₋₃ alkyl, monocyclic aryl, monocyclic heteroaryl (optionally substituted with one or more halogens such as chlorine), and monocyclic heterocyclyl. More preferably, the nitrogen atom is substituted with a group selected from CN, C₁₋₄ alkyl (optionally substituted with OH, CN, COOH), monocyclic aryl C₁₋₃ alkyl, and monocyclic heteroaryl C₁₋₃ alkyl (preferably piperidinyl C₁₋₃ alkyl). More preferably still, the nitrogen atom is substituted with a group selected from C₁₋₄ alkyl (optionally substituted with OH, CN, COOH), monocyclic aryl C₁₋₃ alkyl, and monocyclic heteroaryl C₁₋₃ alkyl (preferably piperidinyl C₁₋₃ alkyl).

In one embodiment, the nitrogen atom is substituted with monocyclic aryl (preferably phenyl) C₁₋₃ alkyl; preferably, the nitrogen atom is substituted with benzyl or phenylethyl; and, more preferably, the nitrogen atom is substituted with benzyl.

In one embodiment R5 is H, halogen, OH or C₁₋₄ alkyl. Preferably, R5 is H.

In another embodiment, R6 is selected from aryl, heteroaryl, and heterocyclyl, each of which may be substituted or unsubstituted. Preferably, R6 is selected from monocyclic aryl (such as phenyl), monocyclic heteroaryl (such as pyridyl), and heterocyclyl, each of which may be substituted or unsubstituted. In one embodiment, R6 is an unsubstituted aryl. When R6 is a substituted aryl, it is preferably substituted with one or more groups selected from halogen, R6a, OH, OR6a, NH₂, NO₂, NHC(NH)NH₂, NHR6a, NR6aR6b, C(NOH)NH₂, COR6a, COOH, COOR6a, CONH₂, CONHOH, SO₂R6a, SO₂NR6aR6b, wherein R6a and R6b are independently selected from C₁. 6 alkyl, substituted C₁₋₆ alkyl, aryl, heteroaryl, C₃₋₈ cycloalkyl and heterocyclyl,

wherein, when the substituent of R6 is C₁₋₆ alkyl, substituted C₁₋₆ alkyl, aryl, heteroaryl, C₃₋₈ cycloalkyl, heterocyclyl or is a group containing one or more of these moieties, each of these moieties may optionally be substituted with one or more groups selected from OR6c, OH, and CONH₂, wherein R6c and R6d are independently selected from C₁₋₆ alkyl, substituted C₁₋₆ alkyl, aryl, heteroaryl, C₃₋₈ cycloalkyl and heterocyclyl, and wherein, when the substituent of R6 is heteroaryl or heterocyclyl, each of these moieties may optionally be substituted with one or more oxygen atoms.

Preferably, when R6 is a substituted aryl, it is substituted with one or more groups selected from halogen, OH, C₁. 4 alkoxy, CONH₂, C(NOH)NH₂, CONHOH, SO₂—C₁₋₄ alkyl, heterocyclyl (optionally substituted with an oxygen atom), and aryl (optionally substituted with CONH₂). In one embodiment, R6 may be substituted with one or more groups selected from 5-oxo-4,5-dihydro-1,2,4-oxadiazol-3-yl, 3-carbamoylphenyl, 2H-tetrazol-5-yl, C₁₋₄ alkoxy, halogen, OH, CONHOH.

When R6 is a heterocyclyl, it is preferably substituted with an oxygen atom. The substituent of R6 may be 2-oxo-2,3-dihydro-1H-benzo[d]imidazol-5-yl or 2-oxo-2,3-dihydrobenzo[d]oxazol-5-yl.

When R6 is a heteroaryl, it is preferably unsubstituted or substituted with an oxygen atom. For example, the heterocyclyl may contain an N-oxide. In one embodiment, R6 is pyridyl or pyridyl oxide.

In another embodiment, R8 is H, halogen, OH or C₁₋₄ alkyl. Preferably, R8 is H.

### Formula IIb

In a preferred embodiment of compounds having Formula IIb, R1 is selected from H and C₁₋₄ alkyl, and R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. More preferably, R1 is selected from H, methyl and ethyl, and R2 is selected from aryl, heteroaryl, heterocyclyl, and C₃₋₁₀ cycloalkyl each of which may be substituted or unsubstituted. More preferably, R1 is methyl. More preferably, R2 is selected from aryl, heteroaryl, heterocyclyl, and C₅₋₈ cycloalkyl each of which are monocyclic and may be substituted or unsubstituted. More preferably still, R2 is monocyclic aryl such as phenyl and may be substituted or unsubstituted. When R2 is substituted, the substituent may be aryl, C₁₋₄ alkoxy, aryl C₁₋₄ alkoxy or aryloxy. Preferably, the substituent of R2 is aryl, C₁₋₃ alkoxy, aryl C₁₋₃ alkoxy or aryloxy, wherein the aryl is monocyclic and more preferably, phenyl.

When R2 is a monocyclic C₅₋₈ cycloalkyl or aryl, it is preferably unsubstituted. Preferably, R2 is a cyclohexyl, such as an unsubstituted cyclohexyl. When R2 is a monocyclic saturated heterocyclyl, the heterocyclyl ring preferably contains a single heteroatom, such as nitrogen or oxygen. More preferably, the heterocyclyl is six membered, such as a piperidinyl or tetrahydropyranyl group. In one embodiment the heteroatom is a nitrogen heteroatom which may be substituted or unsubstituted. Preferably, the heteroatom in the said heterocyclyl group is at the 4 position relative to the position of attachment of the heterocyclyl group R2 to the urea nitrogen. In one embodiment, the nitrogen atom is substituted with monocyclic aryl (preferably phenyl) C₁₋₃ alkyl.

In an alternative preferred embodiment of compounds having Formula IIb, R1 and R2, together with the N to which they are attached, form a heterocyclyl group which may be substituted or unsubstituted. Preferably, the heterocyclyl is a 5 or 6 membered monocyclic ring and, more preferably, a 5 membered monocyclic ring. In certain embodiments, the said heterocyclyl contains one or two, preferably 1, additional heteroatoms (i.e. in addition to the N). These additional heteroatoms may be, for example, N, O and/or S. Preferably, the heterocyclyl is oxazolidinyl. Preferably, the oxygen atom in the oxazolidinyl is at the 3 position relative to the urea nitrogen. Preferably, the oxazolidinyl is substituted with one, two or three methyl or ethyl groups. More preferably, the oxazolidinyl is substituted with two methyl or ethyl groups. More preferably still, the oxazolidinyl is substituted with two methyl groups on the same carbon atom. More preferably, the oxazolidinyl is 4,4-dimethyloxazolidin-3-yl.

In yet another preferred embodiment of compounds having formula IIb, R1 and R2, together with the N to which they are attached, form a heterocyclyl group which may be substituted or unsubstituted. Preferably, the heterocyclyl is a 5 or 6 membered monocyclic ring, more preferably, a 6 membered monocyclic ring. In certain embodiments, the said heterocyclyl contains one or two, preferably 1, additional heteroatoms (i.e. in addition to the N). These additional heteroatoms may be, for example, N, O and/or S. In one embodiment, the heterocyclyl is morpholino. In an alternative embodiment, the heterocyclyl is piperazinyl. In other embodiments, the said heterocyclyl contains no additional heteroatoms (i.e. it contains a single N atom). In one embodiment, the heterocyclyl is piperadinyl. Where the heterocyclyl is substituted, it is preferably substituted with aryl, aryl C₁₋₄ alkyl, C₅₋₆ cycloalkyl, or C₅₋₆ cycloalkyl C₁₋₄ alkyl, wherein the aryl is preferably monocyclic and more preferably phenyl, and the cycloalkyl is preferably cyclohexyl. The alkyl is preferably linear. In one embodiment, the heterocyclyl is substituted with an aryl or an aryl C₁₋₄ alkyl (preferably C₁₋₂ alkyl), wherein the aryl is preferably monocyclic and more preferably phenyl. The aryl may optionally be substituted with one or more halogen.

In compounds having formula IIb, R5 is preferably selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, Rya, halogen, OH, OR5a, SH, SR5a, OCOR5a, SCOR5a, NH₂, NO₂, NHR5a, NR5aR5b, COR5a, CSR5a, CN, COOH, COOR5a, CONH₂, SO₂R5a, SO₃H, SO₂NH₂, CONR5aR5b, SO₂NR5aR5b, wherein R5a and R5b are independently selected from C₁₋₆ alkyl, aryl, heteroaryl, C₃₋₈ cycloalkyl and heterocyclyl, and R5a and R5b, together with the heteroatom to which they are joined, can form heterocyclyl. More preferably, R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, halogen, OH, SH, NH₂, NO₂, CN, COOH, CONH₂, SO₃H, SO₂NH₂. More preferably still, R5 is selected from H, C₁₋₄ alkyl, aryl, heteroaryl, heterocyclyl, C₅₋₈ cycloalkyl, C₁₋₄ alkoxy, aryloxy, heteroaryloxy, heterocyclyloxy, halogen, OH, SH, NH₂, NO₂, CN, COOH, CONH₂, SO₃H, SO₂NH₂, wherein the aryl, heteroaryl, heterocyclyl and C₅₋₄ cycloalkyl groups are monocyclic. Even more preferably, R5 is selected from H, C₁₋₃ alkyl, aryl, heteroaryl, heterocyclyl, C₅₋₈ cycloalkyl, halogen, OH, NH₂, COOH and CONH₂, wherein the aryl, heteroaryl, heterocyclyl and C₅₋₈ cycloalkyl groups are monocyclic. More preferably still, R5 is selected from H, C₁₋₂ alkyl and halogen. Even more preferably, R5 is selected from H and halogen such as F, Cl and Br. In one embodiment, R5 is H.

In compounds having formula IIb, R6 is preferably selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R6a, halogen, OH, OR6a, SH, SR6a, OCOR6a, SCOR6a, NH₂, NO₂, NHR6a, NR6aR6b, COR6a, CSR6a, CN, COOH, COOR6a, CONH₂, SO₂R6a, SO₃H, SO₂NH₂, CONR6aR6b, SO₂NR6aR6b, wherein R6a and R6b are independently selected from C₁₋₆ alkyl, aryl, heteroaryl, C₃₋₈ cycloalkyl and heterocyclyl, and R6a and R6b, together with the heteroatom to which they are joined, can form heterocyclyl. More preferably, R6 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, halogen, OH, SH, NH₂, NO₂, CN, COOH, CONH₂, SO₃H, SO₂NH₂. More preferably still, R6 is selected from H, C₁₋₄ alkyl, aryl, heteroaryl, heterocyclyl, C₅₋₈ cycloalkyl, C₁₋₄ alkoxy, aryloxy, heteroaryloxy, heterocyclyloxy, halogen, OH, SH, NH₂, NO₂, CN, COOH, CONH₂, SO₃H, SO₂NH₂, wherein the aryl, heteroaryl, heterocyclyl and C₅₋₈ cycloalkyl groups are monocyclic. Even more preferably, R6 is selected from H, C₁₋₃ alkyl, aryl, heteroaryl, heterocyclyl, C₅₋₈ cycloalkyl, halogen, OH, NH₂, COOH and CONH₂, wherein the aryl, heteroaryl, heterocyclyl and C₅₋₈ cycloalkyl groups are monocyclic. More preferably still, R6 is selected from H, C₁₋₂ alkyl and halogen. Even more preferably, R6 is selected from H and halogen such as F, CI and Br. In one embodiment, R6 is H.

In compounds having formula IIb, R7 is preferably selected from aryl, heteroaryl, heterocyclyl and C₃₋₈ cycloalkyl each of which may be substituted or unsubstituted. More preferably, R7 is selected from aryl and heteroaryl each of which may be substituted or unsubstituted. In one embodiment, the heteroaryl contains one heteroatom, e.g. an oxygen or nitrogen atom. Preferably, the aryl or heteroaryl is monocyclic. More preferably, the aryl or heteroaryl is a six membered monocyclic ring. In one embodiment, the heteroaryl contains a nitrogen atom which is substituted with an oxygen atom such as oxidopyridyl. In another embodiment, R7 is unsubstituted monocyclic aryl such as phenyl, or monocyclic aryl such as phenyl substituted with one or more groups selected from halogen, C₁₋₂ alkoxy (optionally substituted with one or more halogen), or OH. In a particular embodiment, R7 is unsubstituted monocyclic aryl such as phenyl.

When R7 is substituted, the substituent is preferably one or more groups selected from halogen, C₈₋₄ alkoxy, aryl, heteroaryl, heterocyclyl, OH, CONH₂, NH₂, heterocyclyl C₁₋₄ alkoxy, aryl C₁₋₄ alkoxy, heteroaryl C₁₋₄ alkoxy, NO₂, SO₂NH₂, SO₃, C(NOH)NH₂ and morpholinosulfonyl. Preferably, the aryl, heteroaryl and heterocyclyl are monocyclic. In one embodiment, the aryl, heteroaryl and heterocyclyl are six membered monocyclic rings. In a particular embodiment in which R7 is monocyclic aryl, it may optionally be substituted with aryl or heteroaryl, each of which are monocyclic.

### Formula IIc

In a preferred embodiment of compounds having Formula IIe, R1 is selected from H and C₁₋₄ alkyl, and R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. More preferably, R1 is selected from H, methyl and ethyl, and R2 is selected from aryl, heteroaryl, heterocyclyl, and C₃₋₁₀ cycloalkyl each of which may be substituted or unsubstituted. More preferably, R1 is methyl. More preferably, R2 is selected from aryl, heteroaryl, heterocyclyl, and C₅₋₈ cycloalkyl each of which are monocyclic and may be substituted or unsubstituted. More preferably still, R2 is selected from aryl such as phenyl, saturated heterocyclyl, and C₅₋₈ cycloalkyl each of which are monocyclic and may be substituted or unsubstituted. When R2 is a monocyclic C₅₋₈ cycloalkyl or aryl, it is preferably unsubstituted. Preferably, R2 is a cyclohexyl, such as an unsubstituted cyclohexyl. When R2 is a monocyclic saturated heterocyclyl, the heterocyclyl ring preferably contains a single heteroatom such as nitrogen or oxygen. More preferably, the heterocyclyl is six membered, such as a piperidinyl or tetrahydropyranyl group. In one embodiment, the heteroatom is a nitrogen heteroatom which may be substituted or unsubstituted. Preferably, the heteroatom in the said heterocyclyl group is at the 4 position relative to the position of attachment of the heterocyclyl group R2 to the urea nitrogen. In one embodiment, the nitrogen atom is substituted with monocyclic aryl (preferably phenyl) C₁₋₃ alkyl.

In an alternative preferred embodiment of compounds having Formula II; R1 and R2, together with the N to which they are attached, form a heterocyclyl group which may be substituted or unsubstituted. Preferably, the heterocyclyl is a 5 or 6 membered monocyclic ring and, more preferably, a 5 membered monocyclic ring. In certain embodiments, the said heterocyclyl contains one or two, preferably 1, additional heteroatoms (i.e. in addition to the N). These additional heteroatoms may be, for example, N, O and/or S. Preferably, the heterocyclyl is oxazolidinyl. Preferably, the oxygen atom in the oxazolidinyl is at the 3 position relative to the urea nitrogen. Preferably, the oxazolidinyl is substituted with one, two or three methyl or ethyl groups. More preferably, the oxazolidinyl is substituted with two methyl or ethyl groups. More preferably still, the oxazolidinyl is substituted with two methyl groups on the same carbon atom. More preferably, the oxazolidinyl is 4,4-dimethyloxazolidin-3-yl.

In yet another preferred embodiment of compounds having formula IIc, R1 and R2, together with the N to which they are attached, form a heterocyclyl group which may be substituted or unsubstituted. Preferably, the heterocyclyl is a 5 or 6 membered monocyclic ring, more preferably, a 6 membered monocyclic ring. In certain embodiments, the said heterocyclyl contains one or two, preferably 1, additional heteroatoms (i.e. in addition to the N). These additional heteroatoms may be, for example, N, O and/or S. In one embodiment, the heterocyclyl is morpholino. In an alternative embodiment, the heterocyclyl is piperazinyl. In other embodiments, the said heterocyclyl contains no additional heteroatoms (i.e. it contains a single N atom). In one embodiment, the heterocyclyl is piperadinyl. Where the heterocyclyl is substituted, it is preferably substituted with aryl, aryl C₁₋₄ alkyl, C₅₋₆ cycloalkyl, or C₅₋₆ cycloalkyl C₁₋₄ alkyl, wherein the aryl is preferably monocyclic and more preferably phenyl, and the cycloalkyl is preferably cyclohexyl. The alkyl is preferably linear. In one embodiment, the heterocyclyl is substituted with an aryl or an aryl C₁₋₄ alkyl (preferably C₁₋₂ alkyl), wherein the aryl is preferably monocyclic and more preferably phenyl. The aryl may optionally be substituted with one or more halogen.

In compounds having formula IIc, R5 is preferably selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NO₂, NHR5a, NR5aR5b, COR5a, CSR5a, CN, COOH, COOR5a, CONH₂, SO₂R5a, SO₃H, SO₂NH₂, CONR5aR5b, SO₂NR5aR5b, wherein R5a and R5b are independently selected from C₁₋₆ alkyl, aryl, heteroaryl, C₃₋₈ cycloalkyl and heterocyclyl, and R5a and R5b, together with the heteroatom to which they are joined, can form heterocyclyl. More preferably, R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, halogen, OH, SH, NH₂, NO₂, CN, COOH, CONH₂, SO₃H, SO₂NH₂. More preferably still, R5 is selected from H, C₁₋₄ alkyl, aryl, heteroaryl, heterocyclyl, C₅₋₈ cycloalkyl, C₁₋₄ alkoxy, aryloxy, heteroaryloxy, heterocyclyloxy, halogen, OH, SH, NH₂, NO₂, CN, COOH, CONH₂, SO₃H, SO₂NH₂, wherein the aryl, heteroaryl, heterocyclyl and C₅₋₈ cycloalkyl groups are monocyclic. Even more preferably, R5 is selected from H, C₁₋₃ alkyl, aryl, heteroaryl, heterocyclyl, C₅₋₈ cycloalkyl, halogen, OH, NH₂, COOH and CONH₂, wherein the aryl, heteroaryl, heterocyclyl and C₅₋₈ cycloalkyl groups are monocyclic. More preferably still, R5 is selected from H, C₁₋₂ alkyl and halogen. Even more preferably, R5 is selected from H and halogen such as F, Cl and Br. In one embodiment, R5 is H.

In compounds having formula IIc, R6 is preferably selected from aryl, heteroaryl, heterocyclyl and C₃₋₈ cycloalkyl each of which may be substituted or unsubstituted. More preferably, R6 is selected from aryl and heteroaryl each of which may be substituted or unsubstituted. In one embodiment, the heteroaryl contains one heteroatom, e.g. an oxygen or nitrogen atom. Preferably, the aryl or heteroaryl is monocyclic. More preferably, the aryl or heteroaryl is a six membered monocyclic ring. In one embodiment, the heteroaryl contains a nitrogen atom which is substituted with an oxygen atom such as oxidopyridyl. In another embodiment, R6 is unsubstituted monocyclic aryl such as phenyl, or monocyclic aryl such as phenyl substituted with one or more groups selected from halogen, C₁₋₂ alkoxy (optionally substituted with one or more halogen), or OH. In a preferred embodiment, R6 is unsubstituted aryl and, preferably, a monocyclic aryl such as phenyl.

When R6 is substituted, the substituent is preferably one or more groups selected from halogen, C₁₋₄ alkoxy, aryl, heteroaryl, heterocyclyl, OH, CONH₂, NH₂, heterocyclyl C₁₋₄ alkoxy, aryl C₁₋₄ alkoxy, heteroaryl C₁₋₄ alkoxy, NO₂, SO₂NH₂, SO₃, C(NOH)NH₂ and morpholinosulfonyl. Preferably, the aryl, heteroaryl and heterocyclyl are monocyclic. In one embodiment, the aryl, heteroaryl and heterocyclyl are six membered monocyclic rings. In a particular embodiment in which R6 is monocyclic aryl, it may optionally be substituted with one or more groups selected from halogen, OH, C₁₋₃ alkoxy, aryl (e.g. a monocyclic aryl such as phenyl), heteroaryl (e.g. monocyclic heteroaryl containing one or two nitrogen atoms, or one oxygen atom), heterocyclyl (e.g. piperazinyl, piperadinyl or morpholino) C₁₋₃ alkoxy, aryl (e.g. monocyclic aryl such as phenyl) C₁₋₃ alkoxy, CONH₂, NH₂, NO₂, OCHF₂, SO₂NH₂, morpholinosulfonyl and C(NOH)NH₂.

In one embodiment when R6 is monocyclic aryl such as phenyl, the substituent of R6 is aryl, preferably monocyclic aryl such as phenyl, which may be substituted or unsubstituted. Where it is substituted, preferably it is substituted with CONH₂.

When the substituent of R6 is C₁₋₄ alkoxy, aryl, heteroaryl, heterocyclyl, heterocyclyl C₁₋₄ alkoxy, aryl C₁₋₄ alkoxy, heteroaryl C₁₋₄ alkoxy or SO₃, each of these moieties may optionally be substituted with one or more groups selected from halogen, OH, C₁₋₃ alkoxy (which may be substituted with one or more halogen), CONH₂, CN, NCH₃CH₃, NHCOCH₃, methylhydroxybutyl, and methylhydroxybutynyl.

### Formula IId

In a preferred embodiment of compounds having Formula IId, R1 is selected from H and C₁₋₄ alkyl, and R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may be substituted or unsubstituted. More preferably, R1 is selected from H, methyl and ethyl, and R2 is selected from aryl, heteroaryl, heterocyclyl, and C₃₋₁₀ cycloalkyl each of which may be substituted or unsubstituted. More preferably, R1 is methyl. More preferably, R2 is selected from aryl, heteroaryl, heterocyclyl, and C₅₋₈ cycloalkyl each of which are monocyclic and may be substituted or unsubstituted. More preferably still, R2 is selected from aryl such as phenyl, saturated heterocyclyl, and C₅₋₈ cycloalkyl each of which are monocyclic and may be substituted or unsubstituted. Even more preferably, R2 is aryl, such as phenyl, which is monocyclic and may be substituted or unsubstituted. When R2 is substituted, the substituent is preferably one or more halogen.

In one embodiment, R2 is a cyclohexyl, such as an unsubstituted cyclohexyl. When R2 is a monocyclic saturated heterocyclyl, the heterocyclyl ring preferably contains a single heteroatom such as nitrogen or oxygen. More preferably, the heterocyclyl is six membered, such as a piperidinyl or tetrahydropyranyl group. In one embodiment, the heteroatom is a nitrogen heteroatom which may be substituted or unsubstituted. Preferably, the heteroatom in the said heterocyclyl group is at the 4 position relative to the position of attachment of the heterocyclyl group R2 to the urea nitrogen. In one embodiment, the nitrogen atom is substituted with monocyclic aryl (preferably phenyl) C₁₋₃ alkyl.

In an alternative preferred embodiment of compounds having Formula IId, R1 and R2, together with the N to which they are attached, form a heterocyclyl group which may be substituted or unsubstituted. Preferably, the heterocyclyl is a 5 or 6 membered monocyclic ring and, more preferably, a 5 membered monocyclic ring. In certain embodiments, the said heterocyclyl contains one or two, preferably 1, additional heteroatoms (i.e. in addition to the N). These additional heteroatoms may be, for example, N, O and/or S. Preferably, the heterocyclyl is oxazolidinyl. Preferably, the oxygen atom in the oxazolidinyl is at the 3 position relative to the urea nitrogen. Preferably, the oxazolidinyl is substituted with one, two or three methyl or ethyl groups. More preferably, the oxazolidinyl is substituted with two methyl or ethyl groups. More preferably still, the oxazolidinyl is substituted with two methyl groups on the same carbon atom. More preferably, the oxazolidinyl is 4,4-dimethyloxazolidin-3-yl.

In yet another preferred embodiment of compounds having formula IId, R1 and R2, together with the N to which they are attached, form a heterocyclyl group which may be substituted or unsubstituted. Preferably, the heterocyclyl is a 5 or 6 membered monocyclic ring, more preferably, a 6 membered monocyclic ring. In certain embodiments, the said heterocyclyl contains one or two, preferably 1, additional heteroatoms (i.e. in addition to the N). These additional heteroatoms may be, for example, N, O and/or S. In one embodiment, the heterocyclyl is morpholino. In an alternative embodiment, the heterocyclyl is piperazinyl. In other embodiments, the said heterocyclyl contains no additional heteroatoms (i.e. it contains a single N atom). In one embodiment, the heterocyclyl is piperadinyl. Where the heterocyclyl is substituted, it is preferably substituted with aryl, aryl C₁₋₄ alkyl, C₅₋₆ cycloalkyl, or C₅₋₆ cycloalkyl C₁₋₄ alkyl, wherein the aryl is preferably monocyclic and more preferably phenyl, and the cycloalkyl is preferably cyclohexyl. The alkyl is preferably linear. In one embodiment, the heterocyclyl is substituted with an aryl or an aryl C₁₋₄ alkyl (preferably C₁₋₂ alkyl), wherein the aryl is preferably monocyclic and more preferably phenyl. The aryl may optionally be substituted with one or more halogens.

In compounds having formula IId, R5 is preferably selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NO₂, NHR5a, NR5aR5b, COR5a, CSR5a, CN, COOH, COOR5a, CONH₂, SO₂R5a, SO₃H, SO₂NH₂, CONR5aR5b, SO₂NR5aR5b, wherein R5a and R5b are independently selected from C₁₋₆ alkyl, aryl, heteroaryl, C₃₋₈ cycloalkyl and heterocyclyl, and R5a and R5b, together with the heteroatom to which they are joined, can form heterocyclyl. More preferably, R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, halogen, OH, SH, NH₂, NO₂, CN, COOH, CONH₂, SO₃H, SO₂NH₂. More preferably still, R5 is selected from H, C₁₋₄ alkyl, aryl, heteroaryl, heterocyclyl, C₅₋₈ cycloalkyl, C₁₋₄ alkoxy, aryloxy, heteroaryloxy, heterocyclyloxy, halogen, OH, SH, NH₂, NO₂, CN, COOH, CONH₂, SO₃H, SO₂NH₂, wherein the aryl, heteroaryl, heterocyclyl and C₅₋₈ cycloalkyl groups are monocycle. Even more preferably, R5 is selected from H, C₁₋₃ alkyl, aryl, heteroaryl, heterocyclyl, C₅₋₈ cycloalkyl, halogen, OH, NH₂, COOH and CONH₂, wherein the aryl, heteroaryl, heterocyclyl and C₅₋₈ cycloalkyl groups are monocyclic. More preferably still, R5 is selected from H, C₁₋₂ alkyl and halogen. Even more preferably, R5 is selected from H and halogen such as F, Cl and Br. In one embodiment, R5 is H.

In compounds having formula IId, R7 is preferably selected from aryl, heteroaryl, heterocyclyl and C₃₋₈ cycloalkyl each of which may be substituted or unsubstituted. More preferably, R7 is selected from aryl and heteroaryl each of which may be substituted or unsubstituted. In one embodiment, the heteroaryl contains one heteroatom, e.g. an oxygen or nitrogen atom. Preferably, the aryl or heteroaryl is monocyclic. More preferably, the aryl or heteroaryl is a six membered monocyclic ring. In one embodiment, the heteroaryl contains a nitrogen atom which is substituted with an oxygen atom such as oxidopyridyl. In another embodiment, R7 is unsubstituted monocyclic aryl such as phenyl, or monocyclic aryl such as phenyl substituted with one or more groups selected from halogen, C₁₋₂ alkoxy (optionally substituted with one or more halogen), or OH.

When R7 is substituted, the substituent is preferably one or more groups selected from halogen, C₁₋₄ alkoxy, aryl, heteroaryl, heterocyclyl, OH, CONH₂, NH₂, heterocyclyl C₁₋₄ alkoxy, aryl C₁₋₄ alkoxy, heteroaryl C₁₋₄ alkoxy, NO₂, SO₂NH₂, SO₃, C(NOH)NH₂ and morpholinosulfonyl. Preferably, the aryl, heteroaryl and heterocyclyl are monocyclic. In one embodiment, the aryl, heteroaryl and heterocyclyl are six membered monocycle rings. In a particular embodiment in which R7 is monocyclic aryl, it may optionally be substituted with one or more groups selected from halogen, OH, C₁₋₃ alkoxy, aryl (e.g. a monocyclic aryl such as phenyl), heteroaryl (e.g. monocycle heteroaryl containing one or two nitrogen atoms, or one oxygen atom), heterocyclyl (e.g. piperazinyl, piperadinyl or morpholino) C₁₋₃ alkoxy, aryl (e.g. monocyclic aryl such as phenyl) C₁₋₃ alkoxy, CONH₂, NH₂, NO₂, OCHF₂, SO₂NH₂, morpholinosulfonyl and C(NOH)NH₂. In one embodiment when R7 is monocyclic aryl such as phenyl, the substituent of R7 is aryl (e.g. monocyclic aryl such as phenyl) C₁₋₃ alkoxy.

When the substituent of R7 is C₁₋₄ alkoxy, aryl, heteroaryl, heterocyclyl, heterocyclyl C₁₋₄ alkoxy, aryl C₁₋₄ alkoxy, heteroaryl C₁₋₄ alkoxy or SO₃, each of these moieties may optionally be substituted with one or more groups selected from halogen, OH, C₁₋₃ alkoxy (which may be substituted with one or more halogen), CONH₂, CN, NCH₃CH₃, NHCOCH₃, methylhydroxybutyl, and methylhydroxybutynyl

In an alternative embodiment of the process of the invention, a compound is prepared having Formula I or Formula II:

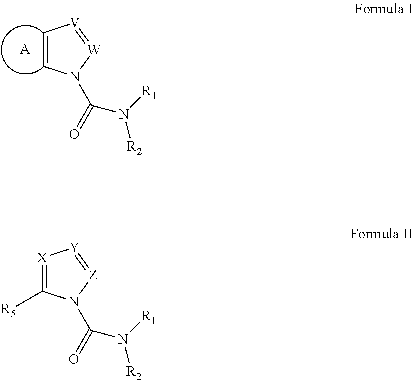

wherein R1, R2, R5, ring A, V, W, X, Y and Z are as defined above;
or a pharmaceutically acceptable salt or ester thereof;
provided that Ring A in compounds having Formula I does not form pyridine, pyrimidine, substituted pyridine or substituted pyrimidine, when R1 and R2, together with the N to which they are attached, form piperidinyl, piperazinyl, substituted piperidinyl or substituted piperazinyl,
provided that Ring A is not unsubstituted benzo, hydroxybenzo, phenoxybenzo, fluorochlorobenzo, chlorobenzo, bromobenzo, nitrobenzo, aminobenzo, cyanobenzo, methylbenzo, trifluoromethylbenzo, trifluoromethylchlorobenzo, phenylketobenzo, phenylhydroxymethylbenzo, cyclohexylthiobenzo, methoxycarbonylbenzo or methoxybenzo,
provided that when R1 or R2 is methyl, the other of R1 or R2 is not 4-chlorobutyl, 4-azidobutyl, or 4-isothiocyanatobutyl, and/or
provided that the compound is not (4-phenyl-1H-imidazol-1-yl)(4-(quinolin-2-ylmethyl)piperazin-1-yl)methanone.

In a particularly preferred embodiment, the compound has the Formula IIa, and the intermediate of Formula II′ has a corresponding structure in which the —CONR1R2 group of Formula Ira is replaced by the H of Formula II′.

In such an embodiment, the compound may, for example, be of Formula IIa, wherein:

R1 is selected from H and C₁₋₄ alkyl,
R2 is selected from C₁₋₆ alkyl, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may optionally be substituted with one or more groups selected from R2a, halogen, OH, OR2a, OCOR2a, SH, SR2a, SCOR2a, NH₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, SO₂R2a, SO₃H, SO₂NH₂, CONR2aR2b, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of R2 is C₁₋₆ alkyl, substituted C₁₋₆ alkyl, aryl, heteroaryl, C₃₋₈₃ cycloalkyl, heterocyclyl or a group containing one or more of these moieties, each of these moieties may optionally be substituted with one or more groups selected from R2c, halogen, OH, OR2c, OCOR2c, SH, SR2c, SCOR2c, NH₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, SO₂R2c, SO₃H, SO₂NH₂, CONR2cR2d, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
R5 is selected from H, R5a, halogen, OH, OR5a, OCOR5a, SH, SR5a, SCOR5a, NH₂, NHR5a, NHSO₂NH₂, NHSO₂R5a, NR5aCOR5b, NHC(NH)NH₂, NHCOR5a, NR5aR5b, COR5a, CSR5a, CN, COOH, COOR5a, CONH₂, CONHOH, CONHR5a, CONHOR5a, C(NOH)NH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl,
R6 is selected from aryl, heteroaryl, heterocyclyl, C₃₋₁₀ cycloalkyl, each of which may optionally be substituted with one or more groups selected from R6a, halogen, OH, OR6a, OCOR6a, SH, SR6a, SCOR6a, NO₂, NH₂, NHR6a, NHSO₂NH₂, NHSO₂R6a, NR6aCOR6b, NHC(NH)NH₂, NHCOR6a, NR6aR6b, COR6a, CSR6a, CN, COOH, COOR6a, CONH₂, CONHOH, CONHR6a, CONHOR6a, C(NOH)NH₂, SO₂R6a, SO₃H, SO₂NH₂, CONR6aR6b, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl, and wherein, when R6 is heteroaryl or heterocyclyl, each of these moieties may optionally be substituted with one or more oxygen atoms,
wherein, when the substituent of R6 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R6c, halogen, OH, OR6c, OCOR6c, SH, SR6c, SCOR6c, NH₂, NHR6c, NHSO₂NH₂, NHSO₂R6c, NR6cCOR6d, NHC(NH)NH₂, NHCOR6c, NR6cR6d, COR6c, CSR6c, CN, COOH, COOR6c, CONH₂, CONHOH, CONHR6e, CONHOR6c, C(NOH)NH₂, SO₂R6c, SO₃H, SO₂NH₂, CONR6cR6d, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl, and wherein, when the substituent of R6 is heteroaryl or heterocyclyl, each of these moieties may optionally be substituted with one or more oxygen atoms, and
R8 is selected from H, R8a, halogen, OH, OR8a, OCOR8a, SH, SR8a, SCOR8a, NH₂, NHR8a, NHSO₂NH₂, NHSO₂R8a, NR8aCOR8b, NHC(NH)NH₂, NHCOR8a, NR8aR8b, COR8a, CSR8a, CN, COOH, COOR8a, CONH₂, CONHOH, CONHR8a, CONHOR8a, C(NOH)NH₂, SO₂R8a, SO₃H, SO₂NH₂, CONR8aR8b, SO₂NR8aR8b, wherein R8a and R8b are independently selected from C₁₋₆ alkyl, substituted C₁₋₆ alkyl, aryl, heteroaryl, C₃₋₈ cycloalkyl and heterocyclyl, or R8a and R8b, together with the heteroatom to which they are joined, can form heterocyclyl.

In particular instances of this preferred embodiment, R1 may be selected from H, methyl and ethyl, and R2 may be selected from aryl, heteroaryl, heterocyclyl, and C₃₋₁₀ cycloalkyl, each of which may be substituted or unsubstituted. R2 may, for example, be selected from fully saturated heterocyclyl, and C₅₋₈ cycloalkyl, each of which are monocyclic and may be substituted or unsubstituted. By way of further example, R2 may be an unsubstituted cyclopentyl or unsubstituted cyclohexyl. As an alternative example, R2 may be a fully saturated heterocyclyl, wherein the heterocyclyl ring contains a single heteroatom, such as nitrogen or oxygen. In such embodiments, the heterocyclyl R2 may be six membered and the heteroatom in the said heterocyclyl group may be at the 4-position relative to the position of attachment of the heterocyclyl group R2 to the urea nitrogen. In particular embodiments, the heteroatom in heterocyclyl R2 may be a nitrogen heteroatom, which may be substituted with a group selected from CN, CONH₂, C(NOH)NH₂, SO₂—C₁₋₄ alkyl, SO₂-aryl, CO-heteroaryl, CO—C₁₋₄ alkyl, COO—C₁₋₄ alkyl, COO-aryl, C₁₋₄ alkyl, aryl C₁₋₃ alkyl, heteroaryl C₁₋₃ alkyl, heterocyclyl C₁₋₃ alkyl, aryl, heteroaryl, and heterocyclyl, wherein the C₁₋₄ alkyl may optionally be substituted with OH, CN, COOH, the SO₂-aryl may optionally be substituted with a C₁₋₄ alkyl or C₁₋₄ haloalkyl, the CO-heteroaryl may optionally be substituted with a heteroaryl or halogen, the heteroaryl C₁₋₃ alkyl may optionally be substituted with COO—C₁₋₃ alkyl, and the heteroaryl may optionally be substituted with one or more halogens. For example, the nitrogen heteroatom in heterocyclyl R2 may be substituted with phenyl C₁₋₃ alkyl.

In particular embodiments of the process of the invention, R6 may be selected from monocyclic aryl, monocyclic heteroaryl, and heterocyclyl, each of which may be substituted or unsubstituted. For example, R6 may be a substituted aryl, wherein said aryl may be substituted with one or more groups selected from halogen, R6a, OH, OR6a, NH₂, NO₂, NHC(NH)NH₂, NHR6a, NR6aR6b, C(NOH)NH₂, COR6a, COOH, COOR6a, CONH₂, CONHOH, SO₂R6a, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl,

wherein, when the substituent of R6 is C₁₋₆ alkyl, substituted C₁₋₆ alkyl, aryl, heteroaryl, C₃₋₈ cycloalkyl, heterocyclyl or is a group containing one or more of these moieties, each of these moieties may optionally be substituted with one or more groups selected from OR6c, OH, and CONH₂, wherein R6c is selected from C₁₋₆ alkyl, substituted C₁₋₆ alkyl, aryl, heteroaryl, C₃₋₈ cycloalkyl and heterocyclyl, and wherein, when the substituent of R6 is heteroaryl or heterocyclyl, each of these moieties may optionally be substituted with one or more oxygen atoms.

In certain embodiments wherein R6 is a substituted aryl, R6 may be substituted with one or more groups selected from halogen, OH, NO₂, C₁₋₄ alkoxy, CONH₂, C(NOH)NH₂, CONHOH, SO₂—C₁₋₄ alkyl, heterocyclyl, and aryl, wherein the heterocyclyl substituent on R6 may optionally be substituted with an oxygen atom and the aryl substituent on R6 may optionally be substituted with CONH₂.

In certain embodiments wherein R6 is a heterocyclyl, R6 is optionally substituted with an oxygen atom. Similarly, in certain embodiments wherein R6 is a monocyclic heteroaryl, R6 is optionally substituted with an oxygen atom.

In particular embodiments of the process of the invention, R8 is H. In certain embodiments, R5 is H. In certain examples of the process of the invention, R5 and R8 are both H.

In a particular group of embodiments, the present invention provides a process for preparing a substituted urea of Formula IIa, or a pharmaceutically acceptable salt or ester thereof, as described above, the process comprising the reaction of an imidazolyl intermediate of Formula II′ having a structure corresponding with Formula IIa in which the —CONR1R2 group of Formula IIa is replaced by the H of Formula II′,

with a carbamoyl halide of the formula: R1R2NC(═O)Hal,
wherein R8 is H;
R1 and R2 can each be independently selected from H, C₁₋₂₀ alkyl, C₁₋₆ alkoxy, aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl-C₁₋₆ alkyl, each of which may be optionally substituted, or R1 and R2, together with the N to which they are attached, can form a heteroaryl or heterocyclyl group, each of which may optionally be substituted, or R1 and R2 can each be independently selected from R1a, halogen, OH, OR1a, OCOR1a, SH, SR1a, SCOR1a, NH₂, NHR1a, NHSO₂NH₂, NHSO₂R1a, NR1aCOR1b, NHCOR1a, NR1 aR1b, COR1a, CSR1a, CN, COOH, COOR1a, CONH₂, CONHOH, CONHR1a, CONHOR1a, SO₂R1a, SO₃H, SO₂NH₂, CONR1aR1b, SO₂NR1aR1b, wherein R1a and R1b are independently selected from optionally substituted C₁₋₆ alkyl, aryl, heteroaryl, C₃₋₈ cycloalkyl and heterocyclyl, or R1a and R1b, together with the heteroatom to which they are joined, can form heterocyclyl,
with the exception that R1 and R2 are not both H;
R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NH₂, NO₂, NHR5a, NHSO₂NH₂, NHSO₂R5a, NR5aCOR5b, NHCOR5a, NHC(NH)NH₂, NR5aR5b, COR5a, CSR5a, CN, COOH, COOR5a, CONH₂, CONHOH, CONHR5a, CONHOR5a, C(NOH)NH₂, CONR5aR5b, SO₂R5a, SO₃H, SO₂NH₂, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl,
wherein, when R5 is C₁₋₆ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R5c, C₁₋₆ alkyl, OH, OR5c, OCOR5c, SH, SR5c, SCOR5c, NH₂, NO₂, NHR5c, NHSO₂NH₂, NHSO₂R5c, NR5cCOR5d, NHCOR5c, NHC(NH)NH₂, NR5cR5d, COR5c, CSR5c, CN, COOH, COOR5c, CONH₂, CONHOH, CONHR5c, CONHOR5c, C(NOH)NH₂, CONR5cR5d, SO₂R5c, SO₃H, SO₂NH₂, SO₂NR5cR5d, wherein R5c and R5d are independently selected from C₁₋₆ alkyl, substituted C₁₋₆ alkyl, aryl, heteroaryl, C₃₋₈ cycloalkyl and heterocyclyl, or R5c and R5d, together with the heteroatom to which they are joined, can form heterocyclyl,
wherein, when the substituent of R5 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R5e, C₁₋₆ alkyl, OH, OR5e, OCOR5e, SH, SR5e, SCOR5e, NH₂, NO₂, NHR5e, NHSO₂NH₂, NHSO₂R5e, NR5eCOR5f, NHCOR5e, NHC(NH)NH₂, NR5eR5f, COR5e, CSR5e, CN, COOH, COOR5e, CONH₂, CONHOH, CONHR5e, CONHOR5e, C(NOH)NH₂, CONR5eR5f, SO₂R5e, SO₃H, SO₂NH₂, SO₂NR5eR5f, wherein R5e and R5f are independently selected from C₁₋₆ alkyl, substituted C₁₋₆ alkyl, aryl, heteroaryl, C₃₋₈ cycloalkyl and heterocyclyl, or R5e and R5f, together with the heteroatom to which they are joined, can form heterocyclyl;
R6 is selected from C₁₋₆ alkyl, aryl, heteroaryl, heterocyclyl, C₁₋₆ alkoxy, aryloxy, heteroaryloxy, heterocyclyloxy, R6a, halogen, OH, OR6a, SH, SR6a, OCOR6a, SCOR6a, NH₂, NO₂, NHR6a, NHSO₂NH₂, NHSO₂R6a, NR6aCOR6b, NHCOR6a, NHC(NH)NH₂, NR6aR6b, COR6a, CSR6a, CN, COOH, COOR6a, CONH₂, CONHOH, CONHR6a, CONHOR6a, C(NOH)NH₂, CONR6aR6b, SO₂R6a, SO₃H, SO₂NH₂, SO₂NR6aR6b, wherein R6a and R6b are independently selected from C₁₋₆ alkyl, substituted C₁₋₆ alkyl, aryl, heteroaryl, cycloalkyl and heterocyclyl, or R6a and R6b, together with the heteroatom to which they are joined, can form heterocyclyl,
wherein, when R6 is heteroaryl or heterocyclyl, each of these moieties may optionally be substituted with one or more oxygen atoms, and when R6 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R6c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR6c, OCOR6c, SH, SR6c, SCOR6c, NH₂, NO₂, NHR6c, NHSO₂NH₂, NHC(NH)NH₂, NHSO₂R6c, NR6cCOR6d, NHCOR6c, NR6cR6d, COR6c, CSR6c, CN, COOH, COOR6c, CONH₂, CONHR6c, CONHOR6c, CONHOH, C(NOH)NH₂, CONR6cR6d, SO₂R6c, SO₃H, SO₂NH₂, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R6c and R6d, together with the heteroatom to which they are joined, can form heterocyclyl,
wherein, when the substituent of R6 is heteroaryl or heterocyclyl, each of these moieties may optionally be substituted with one or more oxygen atoms, or when the substituent of R6 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R6e, C₁₋₆ alkyl, C₁₋₄ alkoxy, OH, OR6e, OCOR6e, SH, SR6e, SCOR6e, NH₂, NO₂, NHR6e, NHSO₂NH₂, NHC(NH)NH₂, NHSO₂R6e, NR6eCOR6f, NHCOR6e, NR6eR6f, COR6e, CSR6e, CN, COOH, COOR6e, CONH₂, CONHOH, CONHR6e, CONHOR6e, C(NOH)NH₂, CONR6eR6f, SO₂R6e, SO₃H, SO₂NH₂, SO₂NR6eR6f, wherein R6e and R6f are independently selected from C₁₋₆ alkyl, substituted C₁₋₆ alkyl, aryl, heteroaryl, C₃₋₈ cycloalkyl and heterocyclyl, or R6e and R6f, together with the heteroatom to which they are joined, can form heterocyclyl.

In certain embodiments of the process of the invention, for example in the particular group of embodiments mentioned immediately above for the preparation of compounds of Formula IIa, R1 and R2 are not both methyl. In particular embodiments, when R1 or R2 is methyl, the other of R1 or R2 is not 4-chlorobutyl, 4-azidobutyl, or 4-isothiocyanatobutyl. In an embodiment, the substituted urea is not (4-phenyl-1H-imidazol-1-yl)(4-(quinolin-2-ylmethyl)piperazin-1-yl)methanone.

R1 and R2 may, especially in the particular group of embodiments mentioned immediately above for the preparation of compounds of Formula IIa, optionally be substituted in the manner set out in claim 1 of WO 2010074588 A2. In particular, in preferred embodiments, when R1 or R2 is C₁₋₂₀ alkyl, alkoxy, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, C₁₋₆ alkyl, C₃₋₈ cycloalkyl or is a group containing one or more of these moieties, each of these moieties may optionally be substituted with one or more groups selected from R1c, halogen, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₁₀ alkyl, OH, OR1c, OCOR1c, SH, SR1c, SCOR1c, NH₂, NO₂, NHR1c, NHSO₂NH₂, NHSO₂R1c, NR1cCOR1d, NHC(NH)NH₂, NHCOR1c, NR1cR1d, COR1c, CSR1c, CN, COOH, COOR1c, CONH₂, CONHOH, CONHR1c, CONHOR1c, C(NOH)NH₂, CONR1cR1d, SO₂R1c, SO₃H, SO₂NH₂, SO₂NR1cR1d, wherein R1c and R1d are independently selected from C₁₋₆ alkyl, substituted C₁₋₆ alkyl, aryl, heteroaryl, c, cycloalkyl and heterocyclyl, or R1c and R1d, together with the heteroatom to which they are joined, can form heterocyclyl,

wherein, when the substituent of R1 or R2 is C₁₋₁₀ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₆ alkyl, C₃₋₈ cycloalkyl or is a group containing one or more of these moieties, each of these moieties may optionally be substituted with one or more groups selected from R1e, halogen, C₁₋₁₀ alkyl, OH, OR1e, OCOR1e, SH, SR1e, SCOR1e, NH₂, NO₂, NHR1e, NHSO₂NH₂, NHSO₂R1e, NR1eCOR1f, NHC(NH)NH₂, NHCOR1e, NR1eR1f, COR1e, CSR1e, CN, COOH, COOR1e, CONH₂, CONHOH, CONHR1e, CONHOR1e, C(NOH)NH₂, CONR1eR1f, SO₂R1e, SO₃H, SO₂NH₂, SO₂NR1eR1f, wherein R1e and R1 f are independently selected from C₁₋₆ alkyl, substituted C₁₋₆ alkyl, aryl, heteroaryl, C₃₋₈ cycloalkyl and heterocyclyl, or R1e and R1f, together with the heteroatom to which they are joined, can form heterocyclyl,
or R1 and R2, together with the N to which they are attached, can form a heteroaryl or heterocyclyl group, each of which may optionally be substituted with one or more oxygen atoms or one or more groups selected from aryl, heteroaryl, partially or fully saturated heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, R2a, halogen, OH, OR2a, OCOR2a, SH, SR2a, SCOR2a, NH₂, NO₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, CONR2aR2b, SO₂R2a, SO₃H, SO₂NH₂, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of the heteroaryl or heterocyclyl formed by R1 and R2 together is aryl, heteroaryl, heterocyclyl, C₃₋₅ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, or a group containing one or more of these moieties, each of these moieties may optionally be substituted with one or more groups selected from halogen, hydroxyl, C₁₋₆ alkyl, aryl, heteroaryl, heterocyclyl, C₃₋₈ cycloalkyl, C₁₋₄ alkoxy, aryloxy, heteroaryloxy, heterocyclyloxy, C₃₋₅ cycloalkyloxy, aryl C₁₋₄ alkoxy, heteroaryl C₁₋₆ alkoxy, heterocyclyl C₁₋₄ alkoxy, C₃₋₈ cycloalkyl C₁₋₄ alkoxy, R2c, OR2c, OCOR2c, SH, SR2c, SCOR2c, NH₂, NO₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, CONR2cR2d, SO₂R2c, SO₃H, SO₂NH₂, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
wherein, when the substituent of the substituent of the heteroaryl or heterocyclyl formed by R1 and R2 together is C₁₋₆ alkyl, aryl, heteroaryl, heterocyclyl, C₃₋₅ cycloalkyl, C₁₋₆ alkoxy, aryloxy, heteroaryloxy, heterocyclyloxy, C₃₋₈ cycloalkyloxy, aryl C₁₋₄ alkoxy, heteroaryl C₁₋₄ alkoxy, heterocyclyl C₁₋₄ alkoxy, C₃₋₅ cycloalkyl C₁₋₄ alkoxy, or is a group containing one or more of these moieties, each of these moieties may optionally be substituted with one or more groups selected from C₁₋₄ alkoxy, R2e, halogen, OH, OR2e, OCOR2e, SH, SR2e, SCOR2e, NH₂, NO₂, NHR2e, NHSO₂NH₂, NHSO₂R2e, NR2eCOR2f, NHC(NH)NH₂, NR2eR2f, NHCOR2e, COR2e, CSR2e, CN, COOH, COOR2e, CONH₂, CONHOH, CONHR2e, CONHOR2e, C(NOH)NH₂, CONR2eR2f, SO₂R2e, SO₃H, SO₂NH₂, SO₂NR2eR2f, wherein R2e and R2f are independently selected from C₁₋₆ alkyl, substituted C₁₋₆ alkyl, aryl, heteroaryl, C₃₋₈ cycloalkyl and heterocyclyl, or R2e and R2f, together with the heteroatom to which they are joined, can form heterocyclyl.

In certain embodiments of the process of the invention for the preparation of compounds of Formula II, and especially in the particular group of embodiments mentioned immediately above for the preparation of compounds of Formula IIa, the urea compound of Formula II has the following features:

R1 is selected from H and C₁₋₄ alkyl,
R2 is selected from aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may optionally be substituted with one or more groups selected from R2a, halogen, OH, OR2a, OCOR2a, SH, SR2a, SCOR2a, NH₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, SO₂R2a, SO₃H, SO₂NH₂, CONR2aR2b, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of R2 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R2c, halogen, OH, OR2c, OCOR2c, SH, SR2c, SCOR2c, NH₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, SO₂R2c, SO₃H, SO₂NH₂, CONR2cR2d, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
R5 is selected from H, R5a, halogen, OH, OR5a, OCOR5a, SH, SR5a, SCOR5a, NH₂, NHR5a, NHSO₂NH₂, NHSO₂R5a, NR5aCOR5b, NHC(NH)NH₂, NHCOR5a, NR5aR5b, COR5a, CSR5a, CN, COOH, COOR5a, CONH₂, CONHOH, CONHR5a, CONHOR5a, C(NOH)NH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl,
R6 is selected from aryl, heteroaryl, heterocyclyl, C₃₋₁₀ cycloalkyl, each of which may optionally be substituted with one or more groups selected from R6a, halogen, OH, OR6a, OCOR6a, SH, SR6a, SCOR6a, NH₂, NHR6a, NHSO₂NH₂, NHSO₂R6a, NR6aCOR6b, NHC(NH)NH₂, NHCOR6a, NR6aR6b, COR6a, CSR6a, CN, COOH, COOR6a, CONH₂, CONHOH, CONHR6a, CONHOR6a, C(NOH)NH₂, SO₂R6a, SO₃H, SO₂NH₂, CONR6aR6b, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl, and wherein, when R6 is heteroaryl or heterocyclyl, each of these moieties may optionally be substituted with one or more oxygen atoms, and
wherein, when the substituent of R6 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R6c, halogen, OH, OR6c, OCOR6c, SH, SR6c, SCOR6c, NH₂, NHR6c, NHSO₂NH₂, NHSO₂R6c, NR6cCOR6d, NHC(NH)NH₂, NHCOR6c, NR6cR6d, COR6c, CSR6c, CN, COOH, COOR6c, CONH₂, CONHOH, CONHR6c, CONHOR6c, C(NOH)NH₂, SO₂R6c, SO₃H, SO₂NH₂, CONR6cR6d; SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl, and wherein, when the substituent of R6 is heteroaryl or heterocyclyl, each of these moieties may optionally be substituted with one or more oxygen atoms.

In such embodiments, R1 may be selected from H, methyl and ethyl, with R2 selected from aryl, heteroaryl, heterocyclyl, and C₃₋₁₀ cycloalkyl, each of which may be substituted or unsubstituted.

In particular, R2 may be selected from fully saturated heterocyclyl and Cₘ cycloalkyl, each of which are monocyclic and may be substituted or unsubstituted. Preferably, R2 is an unsubstituted cyclopentyl or unsubstituted cyclohexyl.

Alternatively in such embodiments, R2 may be a fully saturated heterocyclyl, wherein the heterocyclyl ring contains a single heteroatom, such as nitrogen or oxygen. Such heterocyclyl may be six membered, the heteroatom in the said heterocyclyl group preferably being at the 4-position relative to the position of attachment of the heterocyclyl group R2 to the urea nitrogen. The said heteroatom at the 4-position may be a nitrogen heteroatom which is substituted with a group selected from CN, CONH₂, C(NOH)NH₂, SO₂—C₁₋₄ alkyl, SO₂-aryl, CO-heteroaryl, CO—C₁₋₄ alkyl, COO—C₁₋₄ alkyl, C₁₋₄ alkyl, aryl C₁₋₃ alkyl, heteroaryl C₁₋₃ alkyl, heterocyclyl C₁₋₃ alkyl, aryl, heteroaryl, and heterocyclyl, wherein the C₁₋₄ alkyl may optionally be substituted with OH, CN, COOH, the SO₂-aryl may optionally be substituted with a C₁₋₄ alkyl or C₁₋₄ haloalkyl, the CO-heteroaryl may optionally be substituted with a heteroaryl or halogen, the heteroaryl C₁₋₃ alkyl may optionally be substituted with COO—C₁₋₃ alkyl, and the heteroaryl may optionally be substituted with one or more halogens. In certain such embodiments, the said nitrogen heteroatom is substituted with phenyl C₁₋₃ alkyl.

In particular embodiments, and especially in the particular group of embodiments mentioned immediately above for the preparation of compounds of Formula IIa, R6 is selected from monocyclic aryl, monocyclic heteroaryl, and heterocyclyl, each of which may be substituted or unsubstituted. In such embodiments, R6 may be a substituted aryl, wherein said aryl is substituted with one or more groups selected from halogen, R6a, OH, OR6a, NH₂, NO₂, NHC(NH)NH₂, NHR6a, NR6aR6b, C(NOH)NH₂, COR6a, COOH, COOR6a, CONH₂, CONHOH, SO₂R6a, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl,

wherein, when the substituent of R6 is C₁₋₆ alkyl, substituted C₁₋₆ alkyl, aryl, heteroaryl, C₃₋₈ cycloalkyl, heterocyclyl or is a group containing one or more of these moieties, each of these moieties may optionally be substituted with one or more groups selected from OR6c, OH, and CONH₂, wherein R6c is selected from C₁₋₆ alkyl, substituted C₁₋₆ alkyl, aryl, heteroaryl, C₃₋₈ cycloalkyl and heterocyclyl, and wherein, when the substituent of R6 is heteroaryl or heterocyclyl, each of these moieties may optionally be substituted with one or more oxygen atoms. In particular, R6 may be a substituted aryl which is substituted with one or more groups selected from halogen, OH, C₁₋₄ alkoxy, CONH₂, C(NOH)NH₂, CONHOH, SO₂—C₁₋₄ alkyl, heterocyclyl, and aryl, wherein the heterocyclyl may optionally be substituted with an oxygen atom and the aryl may optionally be substituted with CONH₂.

In alternative embodiments, R6 is a heterocyclyl which is substituted with an oxygen atom. In yet further embodiments, R6 is a monocyclic heteroaryl (such as pyridyl) which is substituted with an oxygen atom (i.e. N-oxidopyridyl).

In preferred embodiments, Hal in the carbamoyl halide used in the process of the invention represents Cl.

In preferred embodiments of the process of the invention, both R1 and R2 in the carbamoyl halide are other than H.

In particular embodiments, including in the particular group of embodiments mentioned immediately above for the preparation of compounds of Formula IIa, R1 is C₁₋₂₀ alkyl, preferably C₁₋₁₀ alkyl, more preferably C₁₋₆ alkyl, such as methyl. In particular embodiments, the said alkyl is unsubstituted.

In certain embodiments, including in the particular group of embodiments mentioned immediately above for the preparation of compounds of Formula IIa, R2 is C₃₋₁₀ cycloalkyl, preferably C₃₋₈ cycloalkyl, such as cyclohexyl. In particular embodiments, the said cycloalkyl is unsubstituted.

In particular embodiments, including in the particular group of embodiments mentioned immediately above for the preparation of compounds of Formula IIa, R5 is H. In certain embodiments, including in the particular group of embodiments mentioned immediately above for the preparation of compounds of Formula IIa, R6 is heteroaryl. Said heteroaryl R6 may be six-membered. For example, R6 may be pyridyl, such as 2-pyridyl, 3-pyridyl or 4-pyridyl (and particularly 3-pyridyl). In such embodiments, the urea of Formula II may be subjected to a further step of N-oxidation of the pyridine (or other heteroaryl) R6. In particular, the N-oxidation may be conducted using a peroxyacid, such as peracetic acid.

In a preferred embodiment of the present invention, the process of the invention is used for the preparation of 3-(1-(cyclohexyl(methyl)carbamoyl-1H-imidazol-4-yl)pyridine 1-oxide (compound A). In another embodiment, the process of the invention is used for the preparation of N-cyclohexyl-N-methyl-4-(pyridin-3yl)-1H-imidazole-1-carboxamide.

In particular embodiments of the process of the invention, the carbamoyl halide is a carbamoyl chloride, prepared by subjecting an amine R1R2NH to carbamoylation using a phosgene reagent, such as triphosgene.

Such a carbamoylation step may be conducted in dichloromethane, in the presence of a base, such as a carbonate salt (e.g. Na).

In certain embodiments, the carbamoyl chloride is not isolated before addition to the intermediate of Formula II′ or Formula I′. It will be appreciated that the intermediate of Formula II′ or Formula I′ is preferably presented in solution in pyridine in these embodiments. In such embodiments, a ‘telescoped’ or one-pot process may be achieved, which can lead to further enhancements in overall urea product yield.

In particular embodiments of the process of the invention, the intermediate of Formula II′ has a structure according to Formula i:

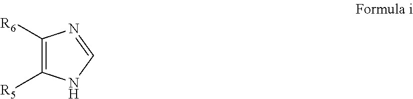

wherein R5 and R6 are as defined above.

In such embodiments, the intermediate of Formula i may in particular be prepared from a mercaptoimidazole having the structure:

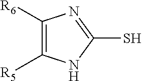

wherein R5 and R6 are as defined above, or an imidazolethione tautomer thereof, using Raney nickel or a nitrate oxidation step (e.g. using a sodium nitrite/nitric acid mixture). An analagous desulphurisation step is described, for example, in Ganellin et al. ((1995), J. Med. Chem. 38, 17) and La Mattina ((1983) J. Heterocyclic Chem. 20, 533). This step may, for example, be conducted in water.

The intermediate of Formula i, especially when produced as described above may, in preferred embodiments, be presented in solution in a solvent, in particular an organic solvent. The solvent may then be chosen so as to enhance the downstream transformation of the intermediate. Thus, in a preferred embodiment, the intermediate of Formula i is transferred to a solution in pyridine, such that it may more readily be used in the process described above. An aspect of the present invention therefore provides an intermediate of Formula i in solution in an organic solvent, wherein Formula i is as defined above. Appropriate solvents include pyridine, isopropyl alcohol, 2-methyltetrahydrofuran, dichloromethane, propionitrile or trifluorotoluene (or mixtures of these solvents, optionally in combination with other common organic solvents used in chemical synthesis).

In turn, where the mercaptoimidazole or imidazolethione tautomer thereof has R5 as H, it may be prepared by treatment of an aminoketone of Formula ii:

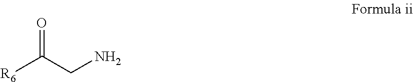

wherein R6 is as defined above, or a salt thereof, with thiocyanate. The thiocyanate may, for example, be an isothiocyanate, such as potassium isothiocyanate. This step may, for example, be conducted in water.

In an alternative embodiment, the intermediate of Formula i, wherein R5 is H, may be prepared by formylation of an aminoketone of Formula ii:

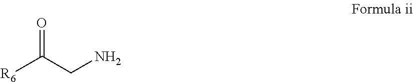

wherein R6 is as defined above, or a salt thereof, followed by reaction of the —NHCHO derivative so formed with an ammonium salt. The formylation may be conducted using an appropriate formyl anhydride, such as aceticformic anhydride, and may for example be conducted in a non-polar solvent such as dichloromethane. The ammonium salt may be organic, such as ammonium acetate, and this reaction may be conducted, for example, in a non-polar solvent such as toluene. This reaction may be aided by addition of para-toluenesulphonic acid, such that a tosylate salt of the intermediate of Formula i is obtained.

In embodiments, the aminoketone or salt of Formula ii is prepared by acid hydrolysis of an azirine derivative of formula iii

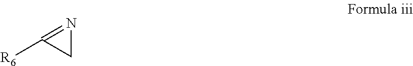

wherein R6 is as defined in claim 33. The acid hydrolysis may, for example, be conducted using concentrated HCl, for example in an alcohol/water solvent (such as ethanol/water). The azirine derivative may have reduced stability, and should only be presented in solution, preferably an acidic solution.

The azirine derivative of formula iii may be prepared by subjecting a ketoxime tosylate derivative of formula iv:

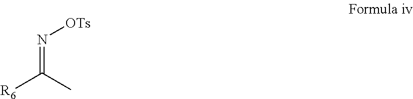

wherein R6 is as defined above and OTs represents toluenesulphonate, to treatment with a base. The base may be organic or inorganic. The organic base may, for example, be an alkoxide salt, such as potassium or sodium t-butoxide, ethoxide or methoxide. Suitable inorganic bases include potassium phosphate and potassium carbonate. The treatment with base may for instance be conducted in an alcoholic solvent, such as t-butanol or methanol, or in an ether solvent such as methyl-t-butyl ether. The inorganic bases may, for example, be presented in dichloromethane.

It will be appreciated by one skilled in the art that the sequence of steps from the ketoxime tosylate to the aminoketone is a form of the Neber rearrangement. Such a reaction sequence is known, for example from Ganellin et al. (1995) and La Mattina (1983) referred to above.

In certain embodiments, the ketoxime tosylate of Formula iv is prepared from the corresponding ketoxime: R6C(═N—OH)CH₃, wherein R6 is as defined above, by reaction with tosyl chloride. Such a reaction may be conducted, for example, using pyridine as solvent.

In embodiments of the process of the invention which include the steps of preparation of the ketoxime tosylate (Formula iv) from the corresponding ketoxime, preparation of the azirine derivative (Formula iii) therefrom, and preparation of the aminoketone (Formula ii) from the azirine derivative, it is preferred that R6 represents an aryl or heteroaryl group, as defined herein.

In certain embodiments, the ketoxime R6C(═N—OH)CH₃ is prepared from the corresponding acetyl derivative of R6: R6-C(═O)CH₃, wherein R6 is as defined above, by reaction thereof with hydroxylamine. This reaction may take place, for example, in an alcoholic solvent such as methanol (optionally with water). An acetate salt, such as sodium acetate, is preferably also used.

In an alternative embodiment, the intermediate of Formula i may be prepared from the acetyl derivative of R6 (R6-C(═O)CH₃) by bromination (for example using HBr, optionally in acetic acid) to R6-C(═O)CH₂Br, followed by treatment with diformalylamide (or its sodium salt) to yield the di-formyl derivative of the aminoketone of Formula ii (i.e. the —N(CHO)(CHO) derivative). This may be readily converted to the formyl derivative, which may then be converted to the intermediate of Formula i by reaction with an ammonium salt, as described above. As a further alternative, the bromoacetyl derivative R6-C(═O)CH₂Br may be treated with an aminating reagent (such as hexamethylenetetramine) to produce the aminoketone of Formula ii.

The present invention also provides a process for preparing an intermediate of Formula i, the process comprising the reaction of an aminoketone of Formula ii, as defined above, or a salt thereof, with thiocyanate, to produce the mercaptoimidazole or imidazolethione tautomer thereof defined above, then the use of Raney nickel or a nitrate oxidation step (e.g. using a sodium nitrite/nitric acid mixture), so as to yield the intermediate of Formula i in solution in a solvent, such as an organic solvent. Preferred solvents include pyridine, IPA (isopropyl alcohol), 2-methyltetrahydrofuran, dichloromethane, propionitrile or trifluorotoluene (or mixtures thereof, optionally in combination with other organic solvents commonly used in chemical synthesis). If the intermediate of Formula i is produced in an organic solvent other than pyridine, it is preferred that a step of solvent exchange is then carried out, such that a pyridine solution is produced.

In a second aspect, the present invention provides a process for the preparation of an aminoketone of Formula ii:

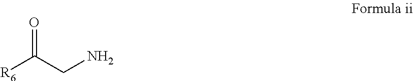

or a salt thereof, wherein R6 is as defined above, the process comprising the tosylation of the corresponding ketoxime: R6C(═N—OH)CH₃, using tosyl chloride in the presence of a first base and in a solvent comprising a C₁₋₆ alcohol, followed by treatment of the resulting ketoxime tosylate, without isolation, with a second base in a solvent comprising a C₁₋₆ alcohol to yield the corresponding azirine derivative of Formula iii:

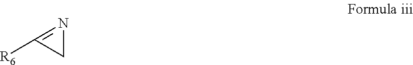

followed by acid hydrolysis of the azirine derivative to yield the aminoketone or salt of Formula ii.

According to the second aspect, the first base, employed during the tosylation step, is preferably a butoxide salt, such as sodium t-butoxide. The solvent used in the tosylation step preferably comprises butanol, such as t-butanol, optionally together with methyl-t-butyl ether. In a preferred embodiment, the base and alcoholic solvent are added to the ketoxime, followed by addition of the tosyl chloride in portions. This approach reduces the potentially disadvantageous exothermicity of the tosylation step. The second base, employed during the production of the azirine derivative, may in particular be a methoxide salt, such as sodium methoxide. this weaker base is more appropriate for the azirine formation. Advantageously, the solvent used during the production of the azirine derivative may be methanol.

The process according to the second aspect is suitable for a ‘telescoped’ or ‘one-pot’ synthesis of the aminoketone of Formula ii from the ketoxime. In such a process, there is no need to isolate the ketoxime tosylate before subjecting it to a Neber rearrangement. Such an approach can lead to an improvement in yield of the aminoketone, and a reduction in the overall reaction time and utilisation of reactor capacity. A yield of aminoketone of 90% has been obtained via this process. The non-telescoped process might typically be expected to yield aminoketone at around 70-85%.

According to the second aspect, the resulting aminoketone of Formula ii may be used to prepare an intermediate of Formula i as defined above, by means of the steps described above.

In a third aspect, there is provided a process for preparing an aminoketone of Formula ii:

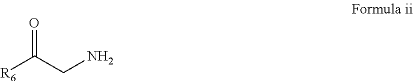

or a salt thereof, wherein R6 is as defined above, the process comprising the reaction of the corresponding acetyl derivative of R6: R6-C(═O)CH₃, with hydroxylamine in a solvent consisting essentially of pyridine, followed by tosylation of the resulting ketoxime, without isolation thereof, using tosyl chloride, followed by treatment of the resulting ketoxime tosylate with a base in a solvent comprising a C₁₋₆ alcohol, to produce the corresponding azirine derivative of Formula iii:

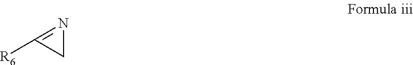

followed by acid hydrolysis of the azirine derivative to yield the aminoketone or salt of Formula ii,

In the third aspect, the reaction between the acetyl derivative and hydroxylamine is conducted in a solvent consisting essentially of pyridine (the meaning of which is the same as defined above in connection with the first aspect). By employing pyridine as solvent (e.g. instead of an alcohol), the resulting ketoxime is obtained in a pyridine solution which can be used directly in the subsequent step (tosylation). This avoids the need for an isolation step (filtration and drying etc.), thereby allowing a telescoped synthesis of the aminoketone and decreasing process time and cost.

Pyridinium salts (e.g. pyridinium HCl when hydroxylamine HCl is used) present in the mixture obtained from the ketoxime preparation step have no detrimental effect on the next steps.

The preferred features of the first aspect, particularly in terms of the definition of R6, are equally preferred in the third aspect. Thus, R6 is in particular embodiments is an optionally substituted aryl or heteroaryl group.

In certain embodiments of the first, second and third aspects, the base used in the conversion of the ketoxime tosylate (Formula iv) to the azirine (Formula iii) comprises 1,8-diazabicyclo[5.4.0]undec-7-ene (hereinafter referred to as DBU).

In certain embodiments of the processes of the invention, when the ketoxime tosylate is converted, via the azirine, to the aminoketone, inorganic salt formation is encountered. Such inorganic salts may, for example, arise from the alkali metal alkoxide used for the azirine formation and the HCl used for hydroysis of the azirine. These inorganic salts can pose problems when trying to isolate the aminoketone by precipitation from an organic solvent such as methanol or ethanol. The inorganic salts have low solubility in the said organic solvent, and hence can be retained on the filter with the aminoketone. Surprisingly, by using DBU, efficient conversion of the ketoxime tosylate can be achieved, yet the salts of DBU which are produced are soluble in e.g. methanol or ethanol and hence can be washed from the aminoketone product. DBU thus leads to a process which yields a high purity aminoketone product, but without the need for a precipitation/filtration step to remove inorganic salt impurities (e.g. by employing MTBE). Other organic bases were tested and were found not to be capable of conversion of the ketoxime tosylate to a useful degree.

In a fourth aspect, there is provided a process for preparing an azirine derivative of Formula iii

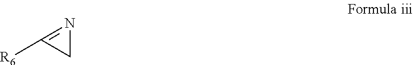

wherein R6 is as defined above, the process comprising subjecting a ketoxime tosylate of Formula iv:

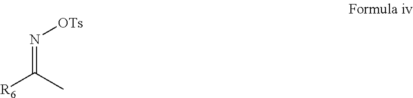

to treatment with a base, wherein the base comprises DBU.

The advantages of using DBU for the conversion of the ketoxime tosylate to the azirine are discussed above in connection with the preceding aspects. In particular, the use of DBU avoids the production of inorganic salts as by-products which have to be removed from downstream products derived from the azirine.

In an embodiment of the fourth aspect, there is provided a process for preparing an aminoketone of Formula ii

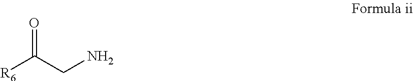

wherein an azirine derivative of Formula iii prepared according to the fourth aspect is subjected to acid hydrolysis.

In another aspect the present invention provides a substituted urea of Formula II or Formula I as defined above, or a pharmaceutically acceptable salt or ester thereof, obtained or obtainable by the processes of the invention as defined above.

Based on the processes of the invention, a number of novel intermediates may be formed which are of use in the synthesis of substituted ureas. Such novel intermediates are also an aspect of the present invention.

In another aspect of the present invention, there is provided a substituted urea compound of Formula II or Formula I as defined above, obtained or obtainable by the process of the first aspect of the invention or by a process in which the process of any of the second, third or fourth aspects is comprised.

In a particular embodiment of this aspect, the substituted urea compound which is obtained or obtainable is 3-(1-(cyclohexyl(methyl)carbamoyl-1H-imidazol-4-yl)pyridine 1-oxide (compound A). In another embodiment, the substituted urea compound which is obtained or obtainable is N-cyclohexyl-N-methyl-4-(pyridin-3yl)-1H-imidazole-1-carboxamide.

The present invention will now be described in more detail by way of example only, with reference to the appended Figures, as follows:

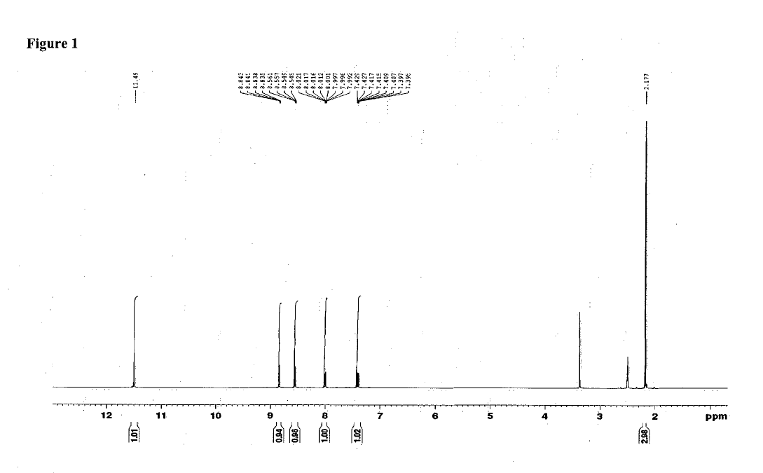

FIG. 1, which shows a ¹H NMR spectrum of a ketoxime R6C(═N—OH)CH₃ used in the process of the invention;

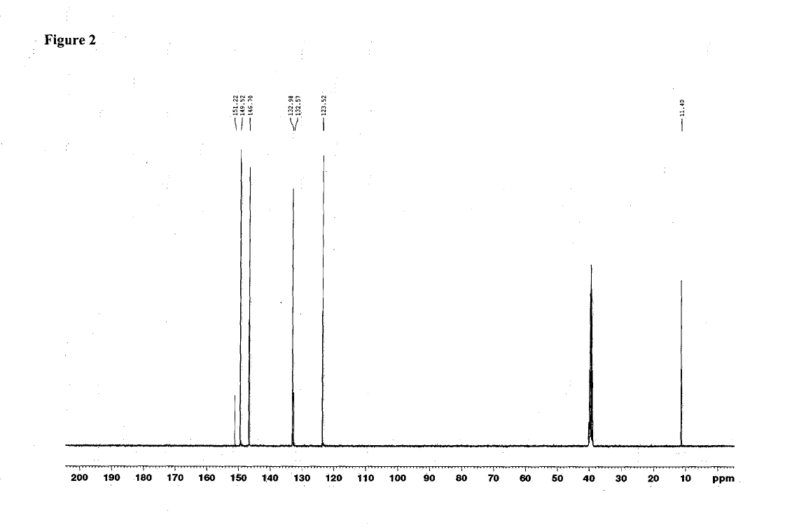

FIG. 2, which shows a ¹³C NMR spectrum of a ketoxime R6C(═N—OH)CH₃ used in the process of the invention (peaks at 151.2, 149.5, 146.7, 133, 132.6, 123.5, 11.4 ppm);

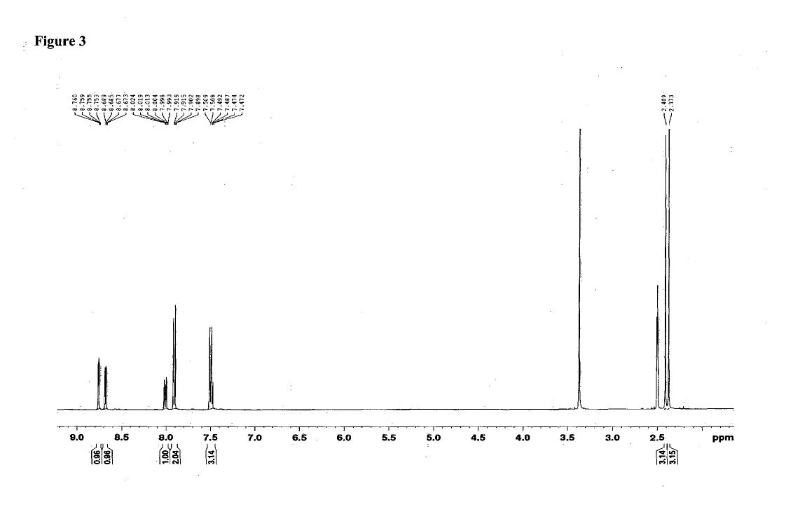

FIG. 3, which shows a ¹H NMR spectrum of a ketoxime tosylate corresponding to the ketoxime of FIGS. 1 and 2;

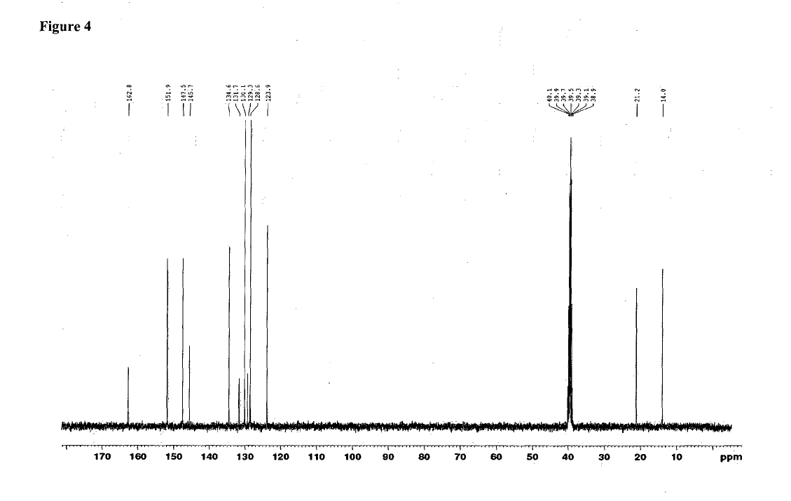

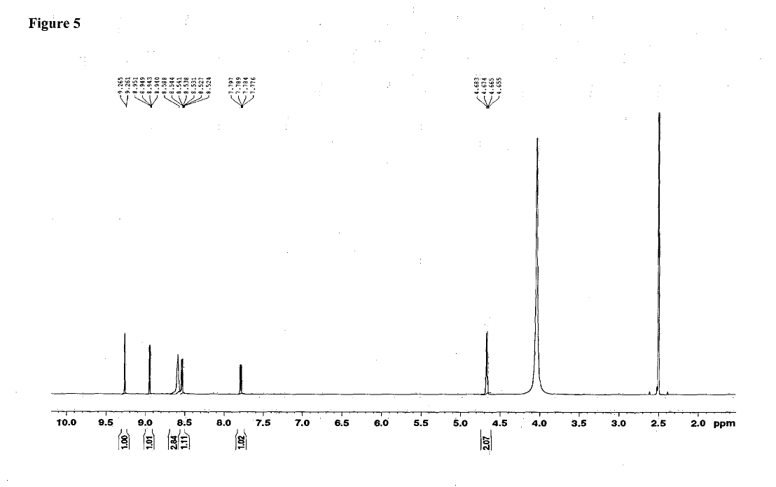

FIG. 4, which shows a ¹³C NMR spectrum of a ketoxime tosylate corresponding to the ketoxime of FIGS. 1 and 2 (peaks at 162.8, 151.9, 147.5, 145.7, 134.6, 131.7, 130.1, 129.3, 128.6, 123.9, 21.2, 14 ppm):

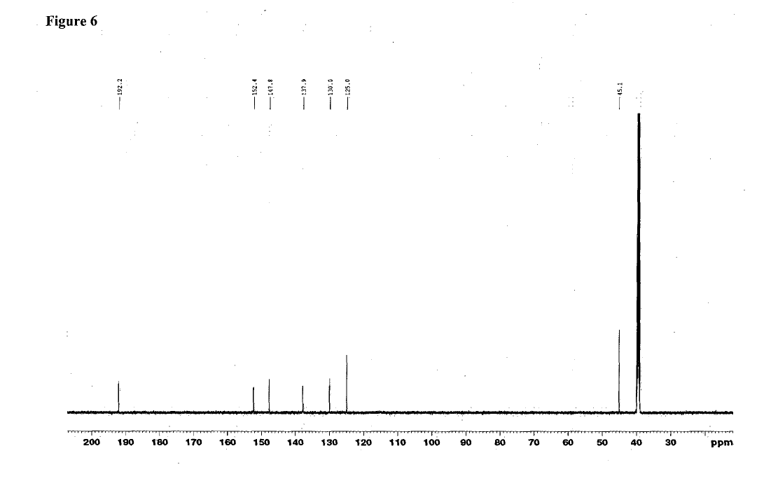

FIG. 5, which shows a ¹H NMR spectrum of an aminoketone of Formula ii, produced from the ketoxime tosylate of FIGS. 3 and 4;

FIG. 6, which shows a ¹³C NMR spectrum of an aminoketone of Formula ii, produced from the ketoxime tosylate of FIGS. 3 and 4 (peaks at 192.2, 152.4, 147.8, 137.9, 130, 125, 45.1 ppm):

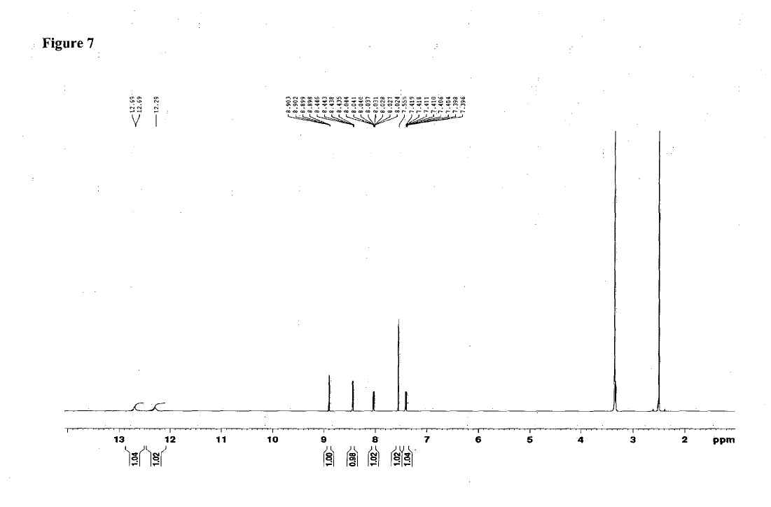

FIG. 7, which shows a ¹H NMR spectrum of a mercaptoimidazole produced from the aminoketone of FIGS. 5 and 6;

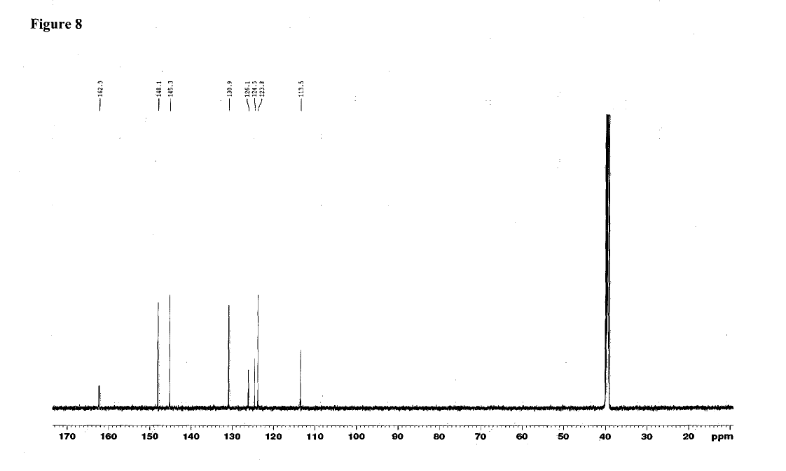

FIG. 8, which shows a ¹³C NMR spectrum of a mercaptoimidazole produced from the aminoketone of FIGS. 5 and 6 (peaks at 162.3, 148.1, 145.3, 130.9, 126.1, 124.5, 123.8, 113.5 ppm);

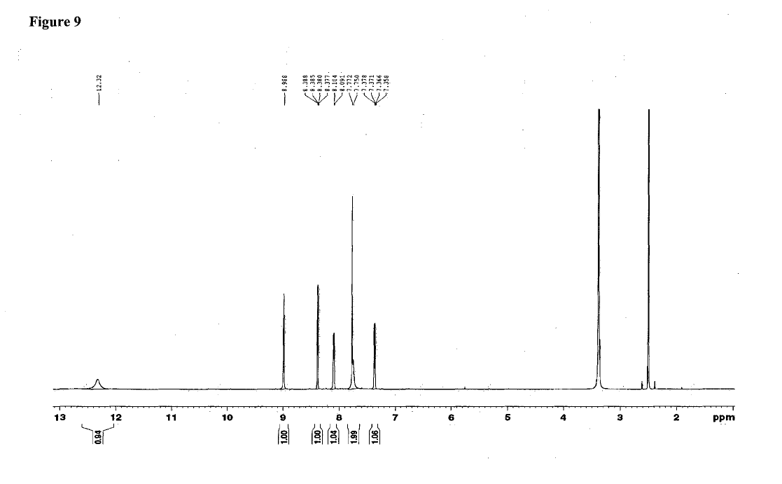

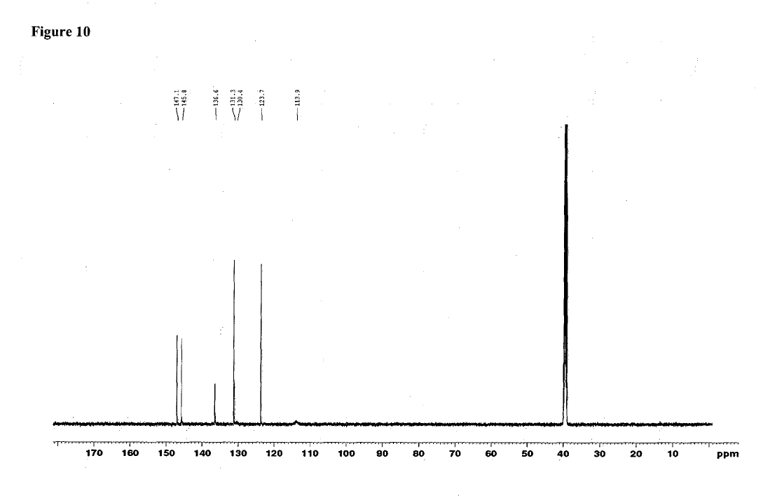

FIG. 9, which shows a NMR spectrum of an intermediate of Formula i produced from the mercaptoimidazole of FIGS. 7 and 8;

FIG. 10, which shows a ¹³C NMR spectrum of an intermediate of Formula i produced from the mercaptoimidazole of FIGS. 7 and 8 (peaks at 147.1, 145.8, 136.6, 131.3, 130.4, 123.7, 113.9 ppm);

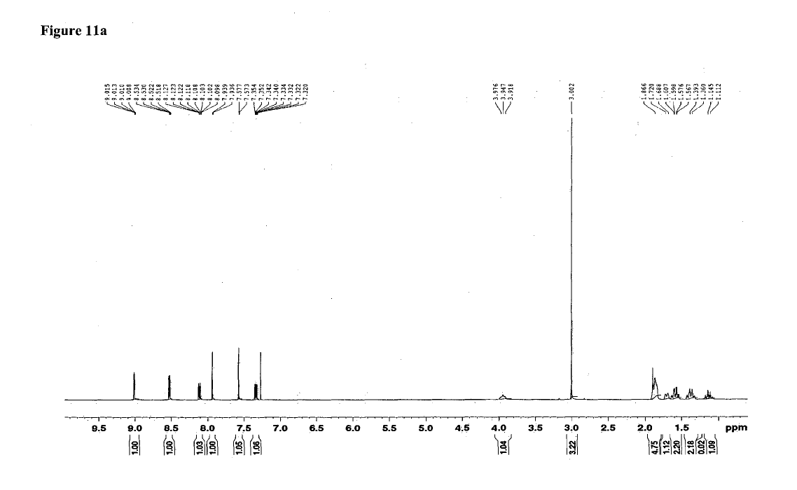

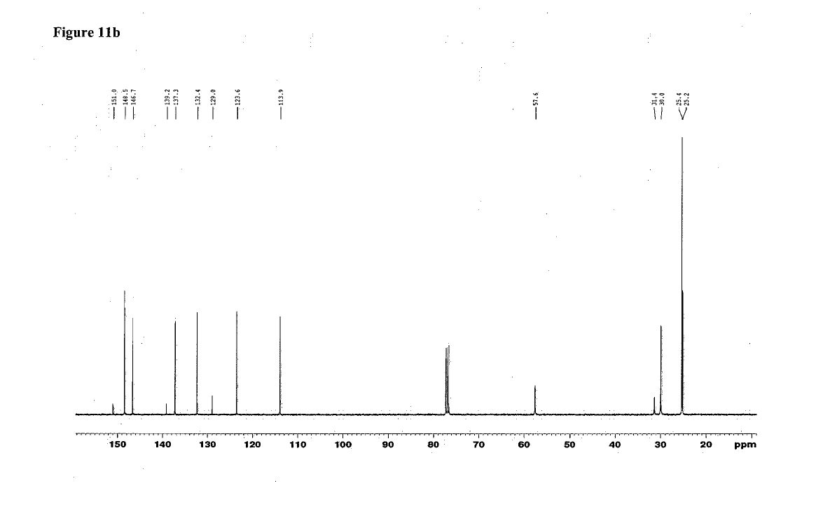

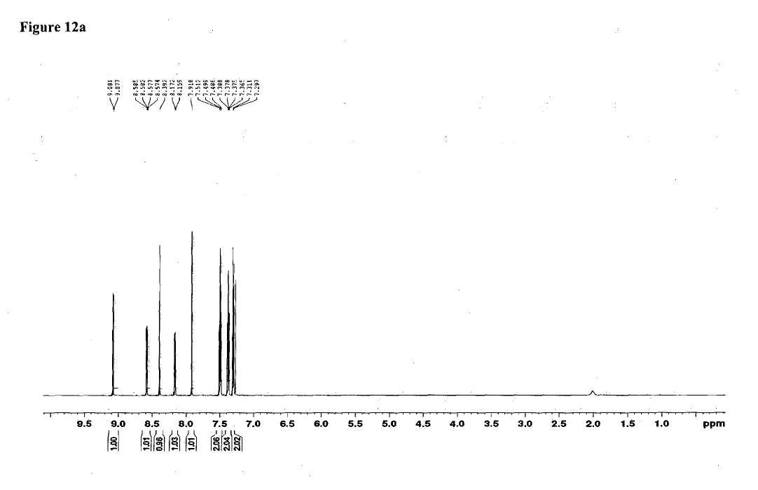

FIG. 11, which shows ¹H (a) and ¹³C (b) NMR spectra of a compound of Formula II (N-cyclohexyl-N-methyl-4-(pyridin-3yl)-1H-imidazole-1-carboxamide) (peaks at 151.0, 148.5, 146.7, 139.2, 137.3, 132.4, 129.0, 123.6, 113.9, 57.6, 31.4, 30.0, 25.4, 25.2) prepared by means of the process of the invention; and

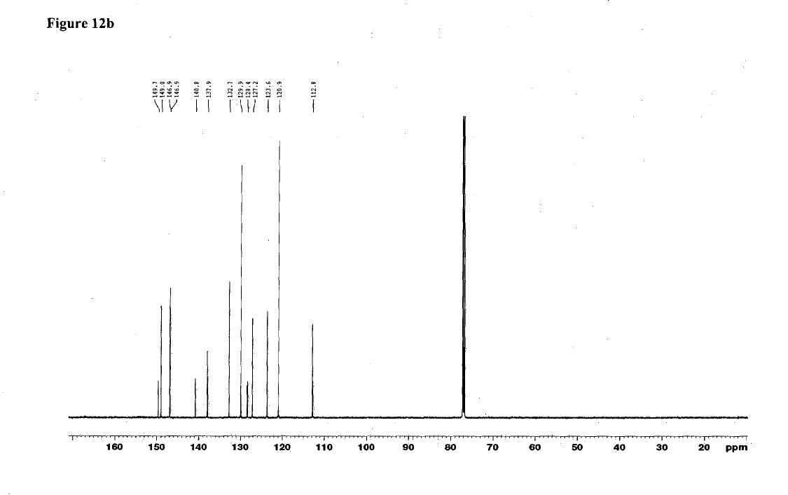

FIG. 12, which shows ¹H (a) and ¹³C (b) NMR spectra of an imidazolylpyridine phenyl carbamate derivative (peaks at 149.7, 149.0, 146.9, 146.9, 140.8, 137.9, 132.7, 129.9, 128.4, 127.2, 123.6, 120.9, 112.8) which can be used to prepare a compound of Formula II.

The Examples which follow illustrate the processes of the present invention by reference to synthesis of the compound N-cyclohexyl-N-methyl-4-(pyridin-3yl)-1H-imidazole-1-carboxamide and its intermediates. NMR spectra of the various intermediates and products were recorded at 20° C., on a Bruker 400 MHz DPX spectrometer with solvent (DMSO) used as internal standard.

### Example 1

### Preparation of 2-amino-1-pyridin-3-yl-ethanone.2HCl

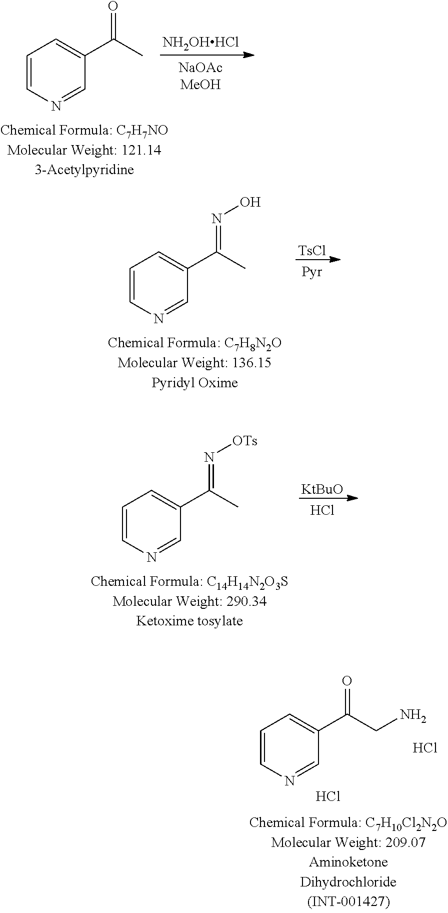

### 1.1 Preparation of 1-pyridin-3-yl-ethanone oxime

3-Acetylpyridine (1.0 wt, 1.00 eq) is charged into the reactor followed by MeOH (6.0 vol). Hydroxylamine hydrochloride (0.69 wt, 1.20 eq) is charged into the reactor. Heat the reaction mixture to reflux and stir for not less than one hour. Charge Sodium Acetate (1.09 wt, 1.61 eq) and stir at reflux for not less than one hour. Cool the mixture to 10° C. in approximately 3 hours and stir at that temperature for not less than one hour. The suspension is filtered and the reactor/cake washed with cold MeOH (1.0 vol). The resultant filtrate is distilled under vacuum at not more than 60° C. to ˜1.5 vol. Water (6.0 vol) is added and the temperature adjusted to 10° C. After stirring the slurry at 10° C. for not less than two hours, the suspension is filtered and the cake washed with cold water (2.0 vol). The cake, comprising the pyridyl oxime, is dried under vacuum.

The purity of the product was ascertained by HPLC, with identity confirmable by NMR (see FIGS. 1 and 2). The yield was consistently around 88-95% in several production runs.

### 1.2 Preparation of 1-pyridin-3-yl-ethanone oxime O-tosylate

Pyridyl Oxime (1.0 wt, 1.00 eq) is charged into the reactor followed by Pyridine (3.7 vol). Cool the reaction mixture to 5° C. Add slowly tosyl chloride (1.54 wt, 1.10 eq). Stir at 25° C. until reaction complete. Charge the reaction mixture, maintaining the temperature below 10° C., into distilled water (23.0 vol) at 0° C. Stir the slurry at 10° C. for not less than two hours. The suspension is filtered and the reactor/cake washed with cold water (5.0 vol). The cake, comprising the ketoxime tosylate, is dried under vacuum at 40° C.

The purity of the product was ascertained by HPLC, with identity confirmable by NMR (see FIGS. 3 and 4). The yield was consistently around 87-95% in several production runs.

### 1.3 Preparation of 2-amino-1-pyridin-3-yl-ethanone.2HCl

To a solution of Potassium tert-Butoxide (0.448 wt, 1.10 eq) in Methanol (4.5 vol) was charged slowly a solution of Ketoxime Tosylate (1.0 wt, 1.00 eq) in Methanol (4.5 vol) maintaining the temperature below 10° C. Heat the reaction mixture to 25° C. Stir at 25° C. for not less than two hours. Charge MTBE (3.0 vol) to the reaction mixture. Cool the mixture to 10° C., stir for 1 hour and filter the suspension while transferring the solution to a different reactor. Wash the cake and reactor with MTBE (0.5 vol) and combine with the filtrate. Charge slowly to the organic layer a solution of 4N HCl (2.58 vol) maintaining the temperature below 10° C. Concentrate the solution under vacuum until ˜1.5 vol.

For conversion of the resulting azirine derivative, charge conc HCl to the slurry and stir at 80° C. for 3 hours. Concentrate under vacuum until ˜1 vol. Charge into the reactor distilled water (1.0 vol) and heat to 50° C. Filter through activated charcoal and wash with distilled water (1.0 vol). Concentrate the aqueous layer under vacuum until ˜1.0 vol. Charge Ethanol (5.0 vol) and continue concentration until ˜1.0 vol. Charge Ethanol (10.0 vol) and heat to reflux. Stir at reflux for 0.5 hour and cool to 5° C. Stir the slurry at 5° C. for not less than two hours. The suspension is filtered and the reactor/cake washed with cold Ethanol (1.0 vol). The cake, comprising the aminoketone, is dried under vacuum.

The purity of the product was ascertained by HPLC, with identity confirmable by NMR (see FIGS. 5 and 6). The yield was consistently around 77-85% in several production runs.

### Example 2

### Preparation of 2-amino-1-pyridin-3-yl-ethanone.2HCl from ketoxime via telescoped process of the invention

This process demonstrates that tosylation of the ketoxime may be performed in alcohol, thereby avoiding the isolation of the tosylate before driving the reaction towards the Neber rearrangement.

A run of the reaction was performed in methanol using 2.1 equiv of t.BuOK to advance not only the completion of the tosylation but, due to the excess, also to take part in the azirine formation. The tosylate formed and it reacted toward the azirine.

In another run, t.BuOH was used as solvent. Tosylation was driven to completion and the following Neber rearrangement was successful to give the expected aminoketone.

At a larger scale replication of this process, the sequence of addition of the reactant and the nature of the base becomes more important. It was determined that t.BuONa/MTBE in t.BuOH is efficient for tosylation of the oxime but is less favourable for the Neber rearrangement. Therefore the Neber rearrangement is preferably conducted in a MeOH/MeONa system.

A preferable approach for a one-pot tosylation and Neber rearrangement according to the present invention is to conduct the tosylation in t.BuOH using t.BuONa/MTBE so that the oxime sodium salt is formed initially, to which the tosyl chloride is added in portions to maintain the temperature around 20-22 degC. The Neber reaction then preferably uses NaOMe/MeOH as base. Upon subsequent hydrolysis of the azirine, an isolated yield of 90% of aminoketone has been achieved without the isolation of the intermediate ketoxime tosylate.

### Example 3

### Preparation of 3-(1H-imidazol-4-yl)-pyridine, an intermediate of Formula i

### 3.1 Preparation of mercaptoimidazole/imidazolethione intermediate

The aminoketone 2-amino-1-pyridin-3-yl-ethanone.2HCl (1.0 wt; 1.00 eq) is charged into the reactor followed by deionized water (3.0 vol). Potassium Thiocyanate (0.535 wt; 1.15 eq) is charged into the reactor. Heat the reaction mixture to 90° C. and stir for not less than 30 minutes. Cool the mixture to 15° C. and stir at that temperature for not less than 30 minutes. The suspension is filtered and the reactor/cake washed with cold deionized water (1.0 vol). The wet cake is added portion wise to a solution of sodium bicarbonate (0.563 wt; 1.40 eq) in deionized water (7.0 vol) at 30° C. The suspension is stirred at 30° C. until no gas evolution is observed and the slurry is cooled to 15° C. After stirring at 15° C. for 1 hour, the suspension is filtered and the reactor/cake washed with deionized water (2.0 vol). The cake, comprising the mercaptoimidazole 4-(pyridin3-yl)-1H-imidazole-2(3H)-thione, is dried under vacuum.

The purity of the product was ascertained by HPLC, with identity confirmable by NMR (see FIGS. 7 and 8). The yield was consistently around 71-79% in several production runs.

### 3.2 Preparation of 3-(1H-imidazol-4-yl)-pyridine

4-(Pyridin-3-yl)-1H-imidazole-2(3H)-thione from 3.1 above (1.0 wt; 1.00 eq) is charged into the reactor followed by deionized water (8 vol). Sodium nitrite (0.58 wt; 1.5 eq) is charged into the reactor. Cool the reaction mixture to 5° C. Add slowly 65% Nitric Acid (1.97 vol; 5 eq). The lines and reactor are rinsed with deionized water (2 vol). Heat the reaction mixture to 35° C. during one hour and stir for not less than 6 hours maintaining the temperature. In some embodiments, the reaction mixture may be heated to 85° C. (e.g. over 3 hours, with stirring for a further 2 hours). Cool the mixture to 15° C. and charge slowly Sodium Carbonate (2.0 wt) (an alternative base is, for example, NaOH). The solution is then heated to 30° C. and saturated with Sodium Chloride (2 wt). To the aqueous layer is charged Isopropanol (4 vol). After stirring for not less than 30 minutes (during which, in some embodiments, the temperature may be increased, for example to 5560° C.), phases are separated, to the aqueous layer Sodium Chloride (2 wt) is charged and the extraction of the aqueous layer is repeated 1 time with IPA (4 vol) and 1 time with IPA (2 vol) (an alternative solvent is, for example, 2-methyl tetrahydrofuran). The mixture is concentrated under vacuum to 2 vol.

The purity of the product was ascertained by HPLC, with identity confirmable by NMR (see FIGS. 9 and 10). The yield was consistently around 84-92% in several production runs.

An important feature of this part of the process is that it allows the production of an intermediate of Formula i in solution in a chosen solvent. Thus, it is possible to isolate the intermediate of Formula i in pyridine so that it may be readily be used in the process of the first aspect of the invention, or in an alternative solvent (IPA in the present example) which may readily be exchanged with pyridine, as described below, or mixed with sufficient pyridine to provide the required solvent ‘consisting essentially of pyridine’, as defined in accordance with the present invention. Particular alternative solvents which may be mixed with pyridine in this manner include 2-methyltetrahydrofuran, dichloromethane, propionitrile and trifluorotoluene.

### Example 4

### Preparation of (N-cyclohexyl-N-methyl-4-(pyridin-3yl)-1H-imidazole-1-carboxamide), a compound of Formula II

### 4.1 Carbamoyl chloride formation

To a solution of Triphosgene (0.80 wt; 0.48 eq) in DCM (6.0 vol) at 10° C. was slowly added a solution of N-Methylcyclohexylamine (0.83 wt; 1.3 eq) in DCM (3.2 vol). Sodium carbonate (1.55 wt; 2.6 eq) was charged and the reaction mixture heated to 25° C. After 3 hours the suspension is filtered and the reactor/cake washed with DCM (1 vol) to produce a solution of N-cyclohexyl-N-methyl carbamoyl chloride.

### 4.2 Urea formation

The IPA solution of imidazolylpyridine from 3.2 is concentrated under vacuum to 2 vol. Pyridine is charged (4 vol) and concentration continued until 2 vol. The solution is filtered and the concentration is repeated two times more until 3 vol. To the resulting pyridine solution of the imidazolylpyridine (3.0 vol; 1.00 eq) at 25° C. is charged the DCM solution of the carbamoyl chloride from 4.1 above. The mixture is heated to 50° C. while distilling. After 30 minutes at 50° C., the reaction mixture is heated to 90° C. in 1 hour continuing the distillation. The mixture is stirred at 90° C. for not less than 1 hour. Cool the mixture to 45° C. in 3 hours. To the suspension is then added Isopropanol (5.2 vol) and after 30 minutes stirring at 45° C. the mixture is cooled to 0° C. in 2 hours. After stirring at 0° C. for not less than 2 hours the suspension is filtered and the reactor/cake washed with cold Isopropanol (1.5 vol), deionized water (10.0 vol) and cold Isopropanol (1.5 vol). The cake, comprising the compound of Formula II, is dried under vacuum.

The purity of the product was ascertained by HPLC, with identity confirmable by NMR (see FIG. 11). The yield was consistently around 86-92% in several production runs.

The urea described in this Example has been produced by the process of the invention in batches of more than 12 kg, with purity of 99.8% (by HPLC). At kg production levels, the overall yield of urea (based on starting from the aminoketone and the R1R2NH amine) is up to approximately 40-60%, and may be improved further. In terms of process efficiency, the use of the process of the invention has the potential to significantly reduce the cost of production of the ureas of Formulae I and II, for example by around 75%.

### Example 5

### 3-(1-(cyclohexyl(methyl)carbamoyl-1H-imidazol-4-yl)pyridine 1-oxide (compound A)

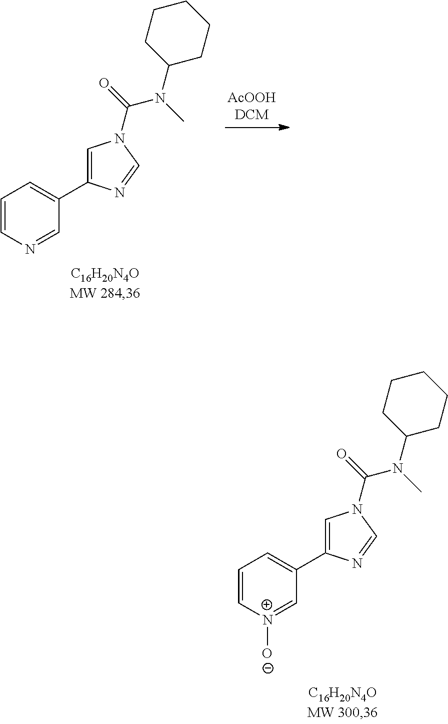 [SMILES: CN(C(=O)n1cnc(-c2cccnc2)c1)C1CCCCC1 ; CN(C(=O)n1cnc(-c2ccc[n+]([O-])c2)c1)C1CCCCC1]

To a solution of N-cyclohexyl-N-methyl-4-(pyridin-3-yl)-1H-imidazole-1-carboxamide in dichloromethane at 25° C. was added peracetic acid (38%; the concentration is not critical, and may be varied) in a single portion. The reaction mixture was then maintained at 25° C. for at least 20 h, whereupon the reaction was washed four times with water (in some embodiments, the water for the extraction step may be supplemented with a small amount (e.g. 1%) of acetic acid, which helps to promote product solubility in the DCM). The dichloromethane solution was then filtered prior to diluting with 2-propanol. Dichloromethane (50%) was then distilled off under atmospheric pressure, whereupon, 2-propanol was charged at the same rate as the distillate was collected. The distillation was continued until >90% of the dichloromethane was collected. The resulting suspension was then cooled to 20° C. and aged for at least 30 min. prior to cooling to 0° C. and aging for a further 60 min. The reaction mixture was then filtered and the product washed with additional 2-propanol, before drying at 50° C. under vacuum to afford the title compound as an off-white crystalline solid.

The purity of the product was ascertained by HPLC, with identity confirmable by NMR. The yield was consistently >80% in several production runs.

### Example 6

### Preparation of (N-cyclohexyl-N-methyl-4-(pyridin-3yl)-1H-imidazole-1-carboxamide) via phenyl carbamate intermediate (Reference Example)

### 6.1 Preparation of phenyl carbamate

3-(1H-Imidazol-4-yl)-pyridine (1) was reacted with phenyl chloroformate (7) in hexane (0.1 mmol), in DCM (0.1 mmol), or preferably in saturated NaHCO₃ (0.1 mmol). Upscale to 10 mmol revealed that phenyl chloroformate can hydrolyse in aqueous NaHCO₃ and 1.5 equiv excess was required to reach improved yield. In toluene (0.689 mmol) the product was isolated in reasonable yield. The structure was confirmed by NMR (FIG. 12) and LCMS (96% purity). Solid NaHCO₃ in THF improved the yield to 99.1% (10 mmol).

Using 2-propanol without any additional base (0.68 mmol) surprisingly resulted in complete conversion of 3-(1H-imidazol-4-yl)-pyridine (1) to the phenyl carbamate HCl salt (8) in 93.2%. Scale up to 10 mmol gave similar results (93.6% yield; 25 mmol 94.9%).

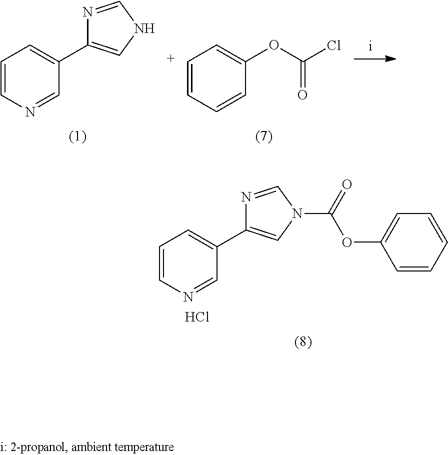 [SMILES: c1cncc(-c2c[nH]cn2)c1 ; O=C(Cl)Oc1ccccc1 ; O=C(Oc1ccccc1)n1cnc(-c2cccnc2)c1 ; Cl]

In order to check the base's melting point the phenyl carbamate base was synthesised from the 3-(1H-imidazol-4-yl)-pyridine (1) and diphenyl carbonate (9) in refluxing 2-Me THE (melting point: 153-155° C.). Similar results were obtained when using toluene (1 mmol), xylene (1 mmol).

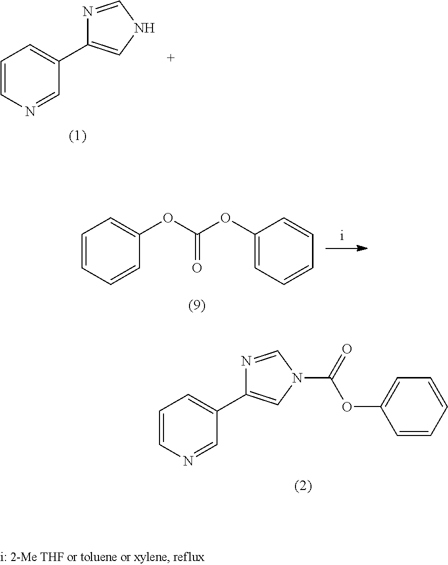 [SMILES: c1cncc(-c2c[nH]cn2)c1 ; O=C(Oc1ccccc1)Oc1ccccc1 ; O=C(Oc1ccccc1)n1cnc(-c2cccnc2)c1]

### 6.2 Transformation of phenyl carbamate to (N-cyclohexyl-N-methyl-4-(pyridin-3yl)-1H-imidazole-1-carboxamide)

A reaction path is shown below. In the reaction of phenyl carbamate as HCl (8) or base (2) conditions were sought wherein the formation of 3-(1H-imidazol-4-yl)-pyridine (1; route a) is significantly lessened or suppressed.

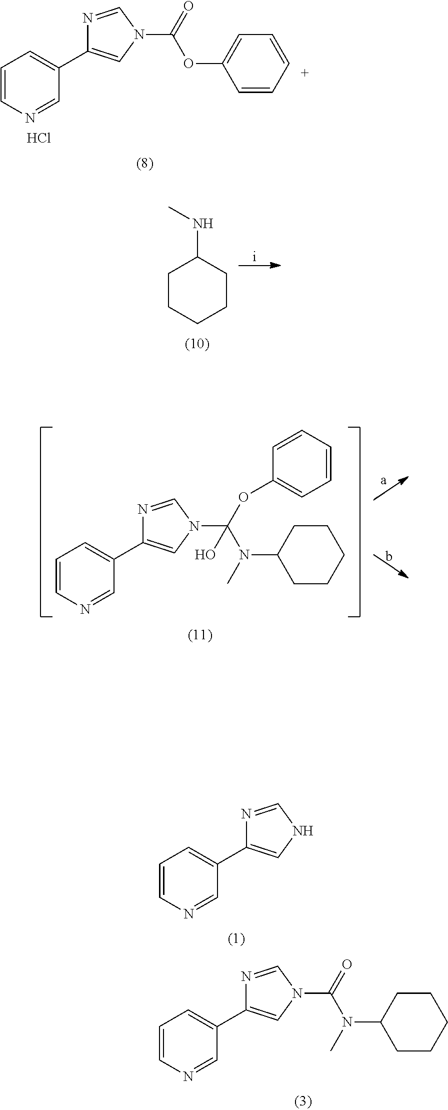

Compound 8 was reacted with 10 in the presence of triethylamine in THF at 25° C. (7.763 mmol; 38.9%). The same reaction can be carried out using DCM as solvent, in THF:water 1:1, in THF:sat aq. NaHCO3, in AcOH/10 creating a buffered environment; in THF using KI as catalyst, in THF and activated charcoal, in 2-propanol, in THF/MgCl₂ system, in MeCN/MgCl₂ system, in MeCN/ZnCl₂ system, in DCM/THF/ZnCl₂ system, in DCM/ZnCl₂ system, in toluene/TEA, in THF/Cu²⁺ system, in trimethyl orthoformate as solvent, in THF/KH₂PO₄ system, in toluene/sat aq. NaHCO₃ system, in THF/DBU system, in THF/EtMgCl system.

### Example 7

### Preparation of Aminoketone Via Alternative Telescoped Route and Using DBU

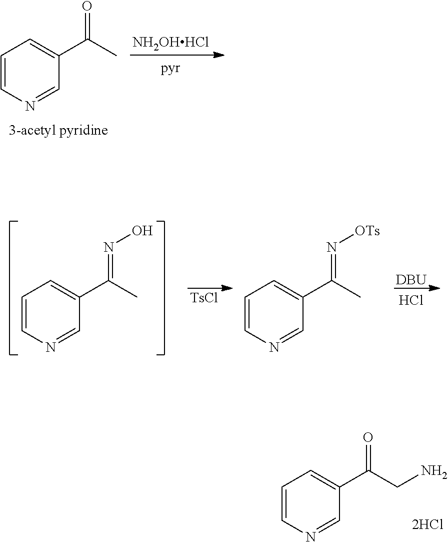

The use of solvents other than pyridine (e.g. methanol) for the first step means that the ketoxime may need to be isolated before tosylation can take place (the latter reaction being particularly favourable in pyridine as solvent). The use of pyridine as solvent for the first step works well, with pyridine acting as a scavenger of HCl. Since the oxime formation generates 1 eq. of water, this should be removed (azeotropic distillation) prior to adding TsCl.

In a typical example, 10 g of acetylpyridine is mixed with 60 ml of pyridine and the mixture os cooled to 5° C. Hydroxylamine HCl (6.02)g is added and the mixture heated to 65° C. After distillation under vacuum, the mixture is cooled to 0° C. Tosyl Cl (18.9 g) is added and the mixture is stirred overnight. The mixture is added to ice/water and stirred. The solid ketoxime tosylate product is filtered and washed with water, then dried under vacuum to obtain a light pink solid (19.6 g, molar yield 82%). Identity was confirmed by NMR.

For the next step, Neber rearrangement and production of aminoketone, a typical example is as follows. The pyridine ketoxime tosylate (18.8 g) in MeOH (150 ml) are charged. DBU (11.6 ml) is added, maintaining the temperature below 20° C. The mixture is stirred at 25° C. until the reaction is complete (orange solution). The reaction is cooled to 0-5° C. and quenched with 4N HCl (48.6 ml), maintaining temperature below 20° C. The mixture is concentrated under vacuum and concentrated HCl is added (44.7 g). The mixture is stirred at 85° C. for 2 hours. The mixture is concentrated under vacuum and water (37.6 ml) is added. After decoloriation (charcoal), and filtration, the solution is concentrated and ethanol is charged, with stirring at 65° C. for 1 hour. After cooling to room temperature, the solid aminoketone product is filtered and washed with ethanol, then dried under vacuum. A light yellow solid (76% molar yield) was obtained. Identity was confirmed by NMR.

### Example 8

### Larger-Scale Production of Aminoketone

The primary objective of this Example is to manufacture and demonstrate a cost-effective pilot scale process for 100 kg Aminoketone Dihydrochloride.

### Step 1:

Batch size: ˜50 kg of 3-acetylpyridine

Expected quantity range: 89 kg to 98 kg of Ketoxime Tosylate

Expected molar yield: 75-82%

Expected quality range: NLT (Not Lower Than) 92% by NMR

### 8.1 Process Outline

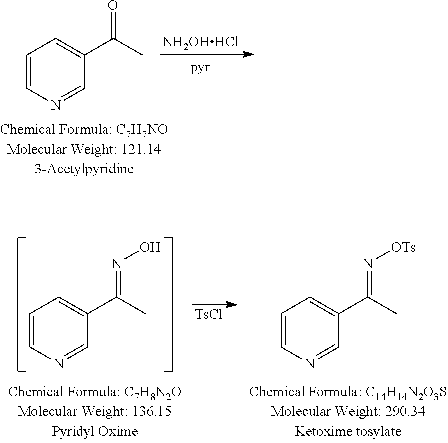

3-Acetylpyridine (1.0 wt, 1.00 eq) and pyridine (6 vol) are mixed together and cooled to 5° C. Hydroxylamine hydrochloride (0.60 wt, 1.05 eq) is slowly added and the mixture heated to 65° C. After 1.5 hour at 65° C. the mixture is concentrated under vacuum until 2 vol of distillates are collected. The mixture is cooled to 0° C. and tosyl chloride (1.89 wt, 1.20 eq) is added in portions. After stirring 12 hours at room temperature the reaction mixture is slowly added to deionized water (18 vol) maintaining the temperature between 15° C. and 25° C. After stirring for 2 hour at 10° C. the suspension is filtered and washed with deionized water (10 vol). The material is dried under vacuum at NMT (Not More Than) 35° C. under nitrogen bleed.

### 8.1.1 Process Detail

1. In reactor A charge 3-Acetylpyridine (1.0 kg) to a reactor
2. Charge Pyridine (6.0 L)
3. Cool the reaction mixture to a temperature between 0° C. and 5° C.
4. Charge Hydroxylamine Hydrochloride (0.60 kg) maintaining the temperature below 10° C. (addition is slightly exothermic)
5. Heat the reaction mixture to a temperature between 65° C. and 70° C.
6. Stir at a temperature between 65° C. and 70° C. for NLT 1.5 hour
7. Concentrate under vacuum until the volume of distillates is 2 L
8. Cool the reactor contents to a temperature between 0° C. and 5° C.
9. Add slowly Tosyl Chloride (1.89 kg) maintaining the temperature below 10° C. (addition is slightly exothermic)
10. Heat to a temperature between 20° C. and 25° C. in NLT 1.5 hr
11. Stir at a temperature between 20° C. and 25° C. for NLT 12 hours
12. In reactor B charge Deionized water (18 L)
13. Cool the contents of reactor B to a temperature between 10° C. and 15° C.
14. Transfer the content of reactor A to reactor B at a rate that the temperature in reactor B is between 15° C. and 25° C. (the addition is exothermic and good temperature control is important to achieve good product precipitation)
15. Rinse reactor A and lines with Pyridine (0.5 L) while transferring to reactor B
16. Adjust temperature between 10° C. and 15° C. and stir for NLT 2 hour
17. Filter and wash reactor and cake with Deionized water (10 L)
18. Dry under vacuum at a temperature between 30° C. and 35° C., under nitrogen sweep until content of deionized water by KF (Karl Fischer) is NMT 1.0%

### 8.1.2 Results

Appearance: Light beige/pinkish crystalline solid

KF: 0.14%

The molar yield is up to 82%, with purity of >92% confirmed by NMR.

### Step 2 Objectives

Batch size: ˜93 kg of Step 1 intermediate

Expected quantity range: 49 kg to 54 kg of Aminoketone Dihydrochloride

Expected molar yield: 72-80%

Expected quality range: NLT 98% by HPLC

### 8.2 Process Outline

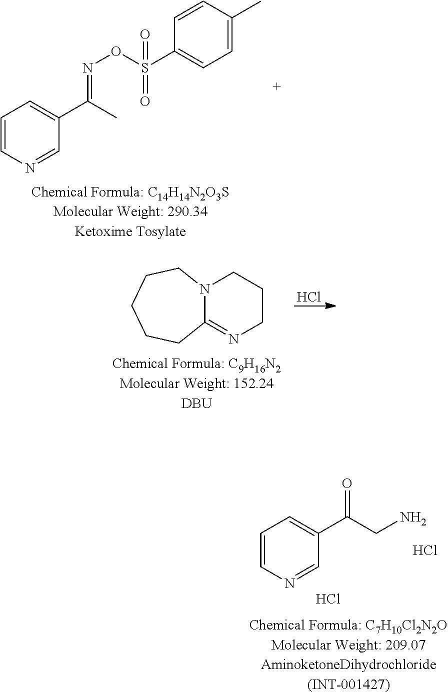 [SMILES: C/C(=N\OS(=O)(=O)c1ccc(C)cc1)c1cccnc1 ; C1CCC2=NCCCN2CC1 ; Cl ; NCC(=O)c1cccnc1 ; Cl ; Cl]

To a mixture of Ketoxime Tosylate (1.0 wt, 1.00 eq) and Methanol (8 vol) is slowly added DBU (0.62 vol, 1.2 eq) maintaining the temperature below 20° C. The mixture is stirred at r.t. until reaction complete. The mixture is cooled to 0/5° C. and quenched with 4N HCl solution (2.58 vol, 3.0 eq) maintaining the temperature below 20° C.

The reaction mixture is concentrated to 1.5 vol under vacuum followed by conc. HCl (2 vol, 7.0 eq) addition. The mixture is heated up to 8590° C. and stirred for 2 hours. The mixture is then concentrated under vacuum to 1.5 vol followed by deionized water (1 vol) addition. The mixture temperature is adjusted to 50° C. and filtered through charcoal cartridge to remove color. The reactor and filter are washed with deionized water (1 vol). and the mixture concentrated under vacuum to 1.5 vol. Ethanol (5 vol) is charged and the mixture concentrated again to 1.5 vol. Ethanol (10 vol) is charged and the slurry stirred at 65° C. for 1 hour. After cooling to r.t. the suspension is filtered and washed with EtOH (1 vol). The material is dried under vacuum at NMT 45° C. until LOD<1.0%.

### 8.2.1 Process Detail

1. Charge Methanol (8 L)
2. Charge Ketoxime Tosylate (1.0 kg)
3. Add slowly DBU (0.62 vol) maintaining the temperature below 20° C. (addition is slightly exothermic, with time the suspension becomes an orange solution)
4. Adjust reaction mixture temperature between 20° C. and 25° C. and stir for NLT 2.5 hours
5. Cool the reaction mixture to a temperature between 0° C. and 5° C.
6. Add slowly a solution of 4N HCl (2.58 L) maintaining the temperature below 20° C. (addition is exothermic. The solution color goes darker. Process maximum volume ˜12 vol)
7. Concentrate under vacuum until ˜1.5 vol at a temperature NMT 60° C. (at the end of distillation dark brown slurry should be observed. Process minimum volume ˜1.5 vol)
8. Charge 37% HCl (2.0 L) to the slurry
9. Heat the mixture to a temperature between 85° C. and 90° C.
10. Stir at a temperature between 85° C. and 90° C. for approximately 2 hours
11. Concentrate under vacuum until ˜1.5 vol at a temperature NMT 60° C. (Note 4)
12. Charge deionized water (1.0 L)
13. Adjust temperature between 55° C. and 50° C.
14. Filter the solution, maintaining the temperature NLT 40° C., through an activated charcoal cartridge while transferring the solution to a different reactor
15. Rinse reactor, filter and lines with distilled deionized water (1.0 L) maintaining the temperature NLT 40° C.
16. Concentrate under vacuum until ˜1.5 vol at a temperature NMT 60° C.
17. Charge Ethanol (5.0 L) and continue distillation until ˜1.5 vol (ethanol used was 95% grade)
18. Charge Ethanol (10.0 L)
19. Heat the slurry to a temperature between 77° C. and 83° C. (reflux should be observed)
20. Stir at a temperature between 77° C. and 83° C. ˜30 minutes
21. Cool to a temperature between 20° C. and 25° C.
22. Stir at a temperature between 20° C. and 25° C. for NLT 2 hours
23. Filter and wash reactor and cake with Ethanol (1.0 L) (wet product density is 0.36)
24. Dry under vacuum at a temperature between 40° C. to 45° C., under nitrogen sweep until LOD NMT (Loss on drying not more than) 1.0% (usually the product is dried in 20 hours)

### 8.2.3 Results

The yield is up to 76%, with purity of up to 99.7% confirmed by NMR.

### Example 9

### Preparation of Additional Ureas of Formula I and II Using Process of the Invention

### 9.1: Alternative imidazole derivatives used for producing urea

### N-cyclohexyl-4-(4-methoxyphenyl)-N-methyl-1H-imidazole-1-carboxamide

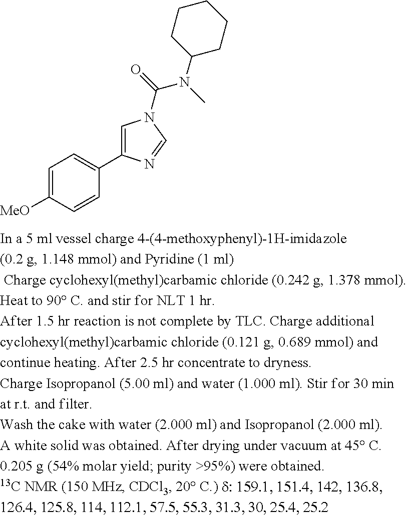 [SMILES: COc1ccc(-c2cn(C(=O)N(C)C3CCCCC3)cn2)cc1]

### N-cyclohexyl-4-(3,5-dinitrophenyl)-N-methyl-1H-imidazole-1-carboxamide

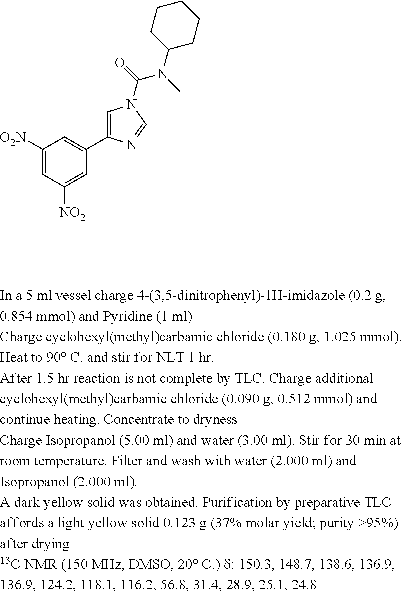 [SMILES: CN(C(=O)n1cnc(-c2cc([N+](=O)[O-])cc([N+](=O)[O-])c2)c1)C1CCCCC1]

### 9.2 Non-Imidazole Structures

### Reference Example

This illustrates that the processes of the invention are not limited to the production of ureas based on imidazole scaffolds.

### 1-benzyl-3-cyclohexyl-1,3-dimethylurea

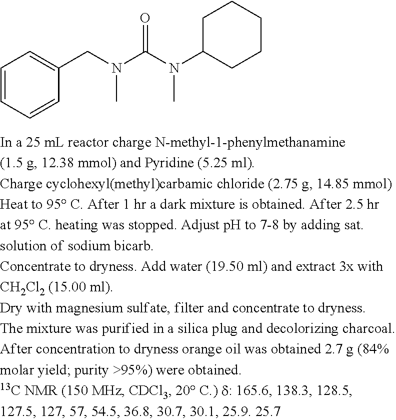 [SMILES: CN(Cc1ccccc1)C(=O)N(C)C1CCCCC1]

### N-cyclohexyl-N-methylpiperidine-1-carboxamide

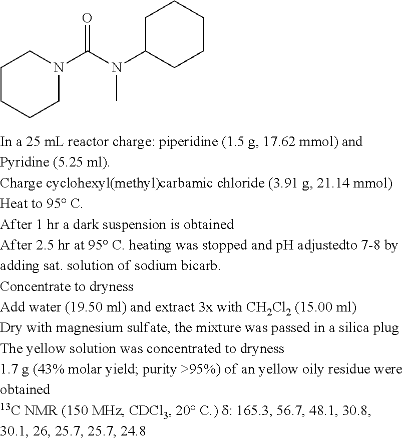 [SMILES: CN(C(=O)N1CCCCC1)C1CCCCC1]

### 9.3 Alternative carbamoyl chlorides used to produce ureas

### N-benzyl-N-methyl-4-(pyridin-3-yl)-1H-imidazole-1-carboxamide

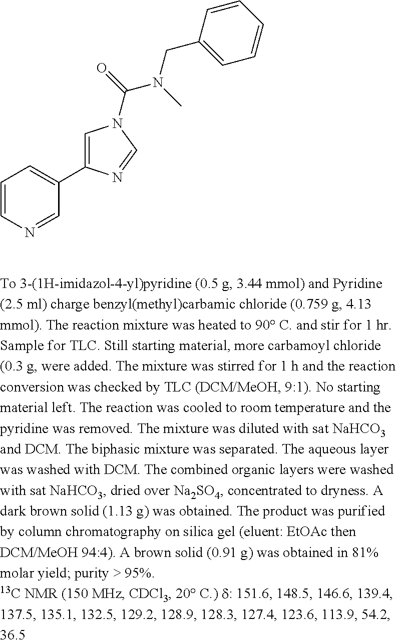 [SMILES: CN(Cc1ccccc1)C(=O)n1cnc(-c2cccnc2)c1]

### Piperidin-1-yl(4-(pyridin-3-yl)-1H-Imidazol-1-yl)methanone

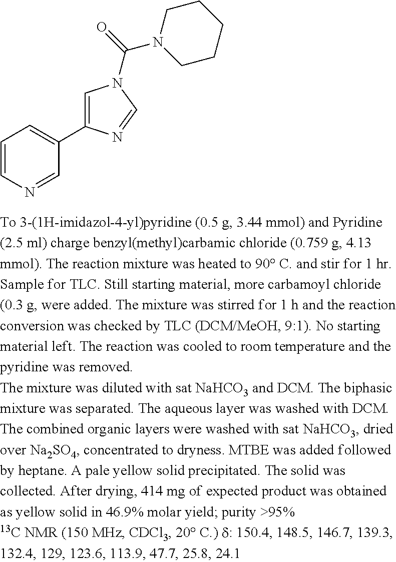 [SMILES: O=C(N1CCCCC1)n1cnc(-c2cccnc2)c1]

### N,N-diethyl-4-(pyridin-3-yl)-1H-imidazole-1-carboxamide

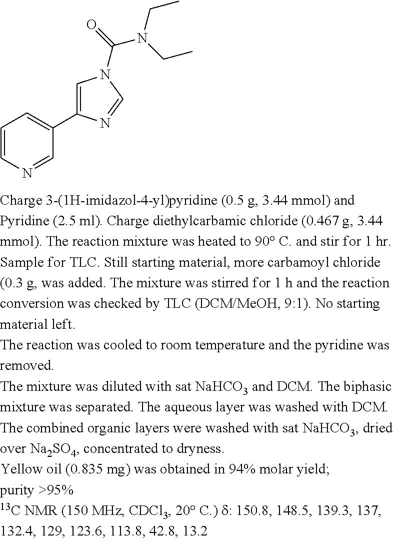 [SMILES: CCN(CC)C(=O)n1cnc(-c2cccnc2)c1]

### (2-methylpyrrolidin-1-yl)(4-(pyridin-3-yl)-1H-imidazol-1-yl)methanone

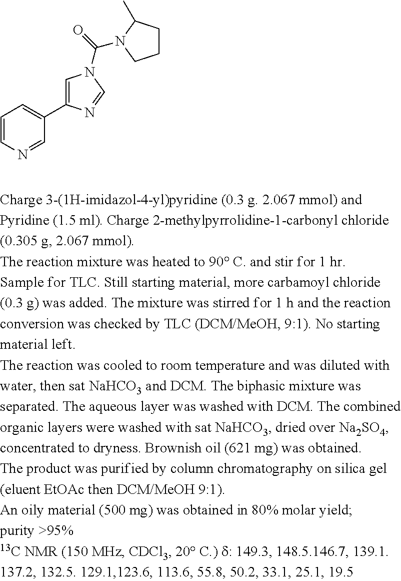 [SMILES: CC1CCCN1C(=O)n1cnc(-c2cccnc2)c1]

### 9.4 Examination of Pyridine Level Used in Solvent for Urea Formation

The goal of this Example was to identify if the method of the invention for the preparation of N-cyclohexyl-N-methyl-4-(pyridin-3yl)-1H-imidazole-1-carboxamide (compound of Formula II) could be carried out in different proportions of solvent/pyridine. Thus a matrix was developed where 4 solvents were going to be tested at different ratios (25; 50 and 75% of pyridine). Below are presented the results under the different conditions (yields are molar yield).

| % | | | | |
|---|---|---|---|---|
| Pyri- | 2-Methyltetra- | Trifluoro- | Dichloro- | |
| dine | hydrofuran | toluene | methane | Propionitrile |
| 75 | Purity: 100% | Purity: 100% | Purity: 100% | Purity: 100% |
| | Yield: 86% | Yield: 86% | Yield: 85% | Yield: 82% |
| | Rx Time: 2 hr | Rx Time: 2 hr | Rx Time: <2 hr | Rx Time: 2 hr |
| 50 | Purity: 100% | Purity: 100% | Purity: 100% | Purity: 100% |
| | Yield: 80% | Yield: 83% | Yield: 77% | Yield: 71% |
| | Rx Time: >2 hr | Rx Time: >2 hr | Rx Time: >2 hr | Rx Time: >2 hr |
| 25 | Purity: 100% | Purity: 100% | Purity: 100% | Purity: 100% |
| | Yield: 56% | Yield: 51% | Yield: 63.3% | Yield: 59.2% |
| | Rx Time: >2 hr | Rx Time: >2 hr | Rx Time: >2 hr | Rx Time: >2 hr |

### Standard Procedure

In a tube reactor charge: 3-(1H-imidazol-4-yl)pyridine (1 g, 6.89 mmol), Pyridine (1 ml), DCM (3.00 ml) and cyclohexyl(methyl)carbamic chloride (1.529 g, 8.27 mmol). Heat to 85° C. until reaction is complete.

Charge Isopropanol (8.00 ml) and stir at room temperature during NLT 3 hr. Filter and wash with water (8.00 ml) and Isopropanol (4.00 ml). Dry under vacuum

As a general conclusion from data analysis, reduced quantities of pyridine present in the reaction mixture (e.g. 25%) lead to the reaction time being longer and yields lower, sometimes due to lower conversion. However the quality is not affected, even at 25% pyridine. It was demonstrated as well that the reaction can be performed without impact on yield and time if ratio of non-pyridine solvent present in reaction mixture is not higher than 25%.

Within the scope of this Example it was also studied the effect of the base catalyst used in the urea formation. Alternatives to pyridine were selected (Triethylamine, Hunig's base and DBU) to be tested in a standard procedure. In all cases the reaction affords the expected product. In the 3 cases problems in the stirring of the reaction were found and in the triethylamine case the quality is affected. The table below summarizes the results:

| Triethylamine | Hunig's base | DBU |
|---|---|---|
| Purity: 94.6% | Purity: 100% | Purity: 100% |
| Yield: 72% | Yield: 56% | Yield: 77% |

The results reported in Examples 9.1 to 9.4 illustrate the general applicability of the processes of the invention in the preparation of ureas of Formula I and II, and the relevant intermediate compounds defined herein. They also illustrate the degree to which the processes described in the other Examples may be varied within the extent of the claims and yet still provide beneficial results.

All documents cited herein are hereby incorporated herein by way of reference in their entirety.

1. A process for preparing a substituted urea compound of Formula II or Formula I, or a pharmaceutically acceptable salt or ester thereof, 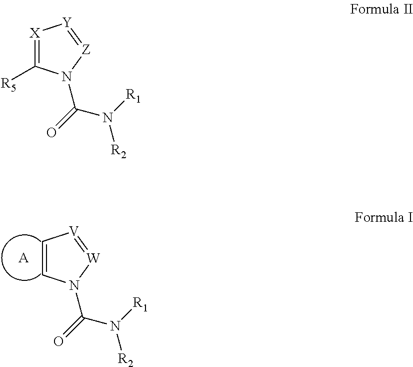
the process comprising the reaction of an intermediate of Formula II′ or Formula I′, 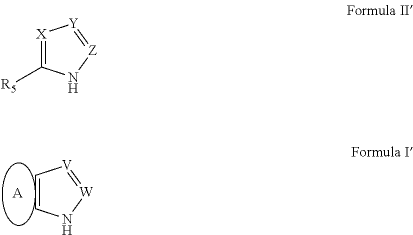
with a carbamoyl halide of the formula: R1R2NC(═O)Hal, in a solvent consisting essentially of pyridine,
wherein Hal represents Cl, F, I or Br,
wherein R1 and R2 can each be independently selected from H, C₁₋₂₀ alkyl, C₁₋₆ alkoxy, aryl, heteroaryl, partially or fully saturated heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, R1a, halogen, OH, OR1a, OCOR1a, SH, SR1a, SCOR1a, NH₂, NHR1a, NHSO₂NH₂, NHSO₂R1a, NR1aCOR1b, NHCOR1a, NR1aR1b, COR1a, CSR1a, CN, COOH, COOR1a, CONH₂, CONHOH, CONHR1a, CONHOR1a, SO₂R1a, SO₃H, SO₂NH₂, CONR1aR1b, SO₂NR1aR1b, wherein R1a and Rib are independently selected from C₁₋₆ alkyl, substituted C₁₋₅ alkyl, aryl, heteroaryl, C₃₋₈ cycloalkyl and heterocyclyl, or R1a and R1b, together with the heteroatom to which they are joined, can form heterocyclyl,
wherein, when R1 or R2 is C₁₋₂₀ alkyl, alkoxy, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl, C₃₋₁₀ cycloalkyl C₁₋₆ alkyl, or is a group containing one or more of these moieties, each of these moieties may optionally be substituted with one or more groups selected from R1c, halogen, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₆ alkyl, OH, OR1c, OCOR1c, SH, SR1c, SCOR1c, NH₂, NO₂, NHR1c, NHSO₂NH₂, NHSO₂R1c, NR1cCOR1d, NHC(NH)NH₂, NHCOR1c, NR1cR1d, COR1c, CSR1c, CN, COOH, COOR1c, CONH₂, CONHOH, CONHR1c, CONHOR1c, C(NOH)NH₂, CONR1cR1d, SO₂R1c, SO₃H, SO₂NH₂, SO₂NR1cR1d, wherein R1c and R1d are independently selected from C₁₋₆ alkyl, substituted C₁₋₆ alkyl, aryl, heteroaryl, C₃₋₈ cycloalkyl and heterocyclyl, or R1c and R1d, together with the heteroatom to which they are joined, can form heterocyclyl,
wherein, when the substituent of R1 or R2 is C₁₋₁₀ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₁₋₆ alkylamino, C₁₋₆ dialkylamino, C₁₋₆ alkyl, C₃₋₈ cycloalkyl or is a group containing one or more of these moieties, each of these moieties may optionally be substituted with one or more groups selected from R1e, halogen, C₁₋₁₀ alkyl, OH, OR1e, OCOR1e, SH, SR1e, SCOR1e, NH₂, NO₂, NHR1e, NHSO₂NH₂, NHSO₂R1e, NR1eCOR1f, NHC(NH)NH₂, NHCOR1e, NR1eR1f, COR1e, CSR1e, CN, COOH, COOR1e, CONH₂, CONHOH, CONHR1e, CONHOR1e, C(NOH)NH₂, CONR1eR1f, SO₂R1e, SO₃H, SO₂NH₂, SO₂NR1eR1f, wherein R1e and R1f are independently selected from C₁₋₆ alkyl, substituted C₁₋₆ alkyl, aryl, heteroaryl, C₃₋₈ cycloalkyl and heterocyclyl, or R1e and R1f, together with the heteroatom to which they are joined, can form heterocyclyl,
with the exception that R1 and R2 are not both H;
or
R1 and R2, together with the N to which they are attached, can form a heteroaryl or heterocyclyl group, each of which may optionally be substituted with one or more oxygen atoms or one or more groups selected from aryl, heteroaryl, partially or fully saturated heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, R2a, halogen, OH, OR2a, OCOR2a, SH, SR2a, SCOR2a, NH₂, NO₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, CONR2aR2b, SO₂R2a, SO₃H, SO₂NH₂, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of the heteroaryl or heterocyclyl formed by R1 and R2 together is aryl, heteroaryl, heterocyclyl, C₃₋₈ cycloalkyl, C₁₋₆ alkyl, aryl C₁₋₆ alkyl, heteroaryl C₁₋₆ alkyl, heterocyclyl C₁₋₆ alkyl, C₃₋₈ cycloalkyl C₁₋₆ alkyl, C₁₋₆ alkoxy, aryloxy, heteroaryloxy, heterocyclyloxy, or a group containing one or more of these moieties, each of these moieties may optionally be substituted with one or more groups selected from halogen, hydroxyl, C₁₋₆ alkyl, aryl, heteroaryl, heterocyclyl, C₃₋₈ cycloalkyl, C₁₋₄ alkoxy, aryloxy, heteroaryloxy, heterocyclyloxy, C₃₋₈ cycloalkyloxy, aryl C₁₋₄ alkoxy, heteroaryl C₁₋₆ alkoxy, heterocyclyl C₁₋₄ alkoxy, C₃₋₈ cycloalkyl C₁₋₄ alkoxy, R2c, OR2c, OCOR2c, SH, SR2c, SCOR2c, NH₂, NO₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, CONR2cR2d, SO₂R2c, SO₃H, SO₂NH₂, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
wherein, when the substituent of the substituent of the heteroaryl or heterocyclyl formed by R1 and R2 together is C₁₋₆ alkyl, aryl, heteroaryl, heterocyclyl, C₃₋₈ cycloalkyl, C₁₋₆ alkoxy, aryloxy, heteroaryloxy, heterocyclyloxy, C₃₋₈ cycloalkyloxy, aryl C₁₋₄ alkoxy, heteroaryl C₁₋₄ alkoxy, heterocyclyl C₁₋₄ alkoxy, C₃₋₈ cycloalkyl C₁₋₄ alkoxy, or is a group containing one or more of these moieties, each of these moieties may optionally be substituted with one or more groups selected from C₁₋₄ alkoxy, R2e, halogen, OH, OR2e, OCOR2e, SH, SR2e, SCOR2e, NH₂, NO₂, NHR2e, NHSO₂NH₂, NHSO₂R2e, NR2eCOR2f, NHC(NH)NH₂, NR2eR2f, NHCOR2e, COR2e, CSR2e, CN, COOH, COOR2e, CONH₂, CONHOH, CONHR2e, CONHOR2e, C(NOH)NH₂, CONR2eR2f, SO₂R2e, SO₃H, SO₂NH₂, SO₂NR2eR2f, wherein R2e and R2f are independently selected from C₁₋₆ alkyl, substituted C₁₋₆ alkyl, aryl, heteroaryl, C₃₋₈ cycloalkyl and heterocyclyl, or R2e and R2f, together with the heteroatom to which they are joined, can form heterocyclyl;
Ring A is selected from aryl, heteroaryl and heterocyclyl moieties, each of which may optionally be substituted with one or more groups selected from halogen, C₁₋₆ alkyl, aryl, heteroaryl, heterocyclyl, C₁₋₆ alkoxy, aryloxy, heteroaryloxy, heterocyclyloxy, Ra, C₁₋₁₀ alkyl, OH, ORa, OCORa, SH, SRa, SCORa, NH₂, NO₂, NHRa, NHSO₂NH₂, NHSO₂Ra, NRaCORb, NHCORa, NHC(NH)NH₂, NRaRb, CORa, CSRa, CN, COOH, COORa, CONH₂, CONHRa, CONHOH, CONHORa, C(NOH)NH₂, CONRaRb, SO₂Ra, SO₃H, SO₂NH₂, SO₂NRaRb, wherein Ra and Rb are independently selected from C₁₋₆ alkyl, substituted C₁₋₆ alkyl, aryl, heteroaryl, C₃₋₈ cycloalkyl and heterocyclyl, or Ra and Rb, together with the heteroatom to which they are joined, can form heterocyclyl,
wherein, when Ring A is substituted with C₁₋₆ alkyl, aryl, heteroaryl, heterocyclyl, C₁₋₆ alkoxy, aryloxy, heteroaryloxy, heterocyclyloxy, C₁₋₁₀ alkyl, C₃₋₈ cycloalkyl or is substituted with a group containing one or more of these moieties, each of these moieties may optionally be substituted with one or more groups selected from halogen, Rc, C₁₋₁₀ alkyl, aryl C₁₋₆ alkyl, heteroaryl C₁₋₆ alkyl, heterocyclyl C₁₋₆ alkyl, OH, ORc, OCORc, SH, SRc, SCORc, NH₂, NO₂, NHRc, NHSO₂NH₂, NHSO₂Rc, NRcCORd, NHCORc, NHC(NH)NH₂, NRcRd, CORc, CSRc, CN, COOH, COORc, CONH₂, CONHOH, CONHRc, CONHORc, C(NOH)NH₂, CONRcRd, SO₂Rc, SO₃H, SO₂NH₂, SO₂NRcRd, wherein Rc and Rd are independently selected from C₁₋₆ alkyl, substituted C₁₋₆ alkyl, aryl, heteroaryl, C₃₋₈ cycloalkyl and heterocyclyl, or Rc and Rd, together with the heteroatom to which they are joined, can form heterocyclyl;
V can be N, CH or C—R3, wherein R3 is halogen, C₁₋₁₀ alkyl, aryl, heteroaryl, heterocyclyl, C₃₋₈ cycloalkyl, C₁₋₆ alkoxy, aryloxy, heteroaryloxy, heterocyclyloxy, R3a, OH, OR3a, SH, SR3a, OCOR3a, SCOR3a, NH₂, NO₂, NHR3a, NHSO₂NH₂, NHSO₂R3a, NR3aCOR3b, NHCOR3a, NHC(NH)NH₂, NR3aR3b, COR3a, CSR3a, CN, COOH, COOR3a, CONH₂, CONHOH, CONHR3a, CONHOR3a, C(NOH)NH₂, CONR3aR3b, SO₂R3a, SO₃H, SO₂NH₂, SO₂NR3aR3b, wherein R3a and R3b are independently selected from C₁₋₆ alkyl, substituted C₁₋₆ alkyl, aryl, heteroaryl, C₃₋₃ cycloalkyl and heterocyclyl, or R3a and R3b, together with the heteroatom to which they are joined, can form heterocyclyl,
wherein, when R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R1c, C₁₋₁₀ alkyl, OH, OR3c, OCOR3c, SH, SR3c, SCOR3c, NH₂, NO₂, NHR3c, NHSO₂NH₂, NHSO₂R3c, NR3cCOR3d, NHCOR3c, NHC(NH)NH₂, NR3cR3d, COR3c, CSR3c, CN, COOH, COOR3c, CONH₂, CONHOH, CONHR3c, CONHOR3c, C(NOH)NH₂, CONR3cR3d, SO₂R3c, SO₃H, SO₂NH₂, SO₂NR3cR3d, wherein R3c and R3d are independently selected from C₁₋₆ alkyl, substituted C₁₋₆ alkyl, aryl, heteroaryl, C₃₋₈ cycloalkyl and heterocyclyl, or R3c and R3d, together with the heteroatom to which they are joined, can form heterocyclyl,
wherein, when the substituent of R3 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, R3e, C₁₋₁₀ alkyl, OH, OR3e, OCOR3e, SH, SR3e, SCOR3e, NH₂, NO₂, NHR3e, NHSO₂NH₂, NHSO₂R3e, NR3eCOR3f, NHCOR3e, NHC(NH)NH₂, NR3eR3f, COR3e, CSR3e, CN, COOH, COOR3e, CONH₂, CONHOH, CONHR3e, CONHOR3e, C(NOH)NH₂, CONR3eR3f, SO₂R3e, SO₃H, SO₂NH₂, SO₂NR3eR3f, wherein R3e and R3f are independently selected from C₁₋₆ alkyl, substituted C₁₋₆ alkyl, aryl, heteroaryl, C₃₋₈ cycloalkyl and heterocyclyl, or R3e and R3f, together with the heteroatom to which they are joined, can form heterocyclyl;
W can be N, CH or C—R4, wherein R4 is halogen, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, R4a, OH, OR4a, SH, SR4a, OCOR4a, SCOR4a, NH₂, NO₂, NHR4a, NHSO₂NH₂, NHSO₂R4a, NR4aCOR4b, NHCOR4a, NHC(NH)NH₂, NR4aR4b, COR4a, CSR4a, CN, COOH, COOR4a, CONH₂, CONHOH, CONHR4a, CONHOR4a, C(NOH)NH₂, CONR4aR4b, SO₂R4a, SO₃H, SO₂NH₂, SO₂NR4aR4b, wherein R4a and R4b are independently selected from C₁₋₆ alkyl, substituted C₁₋₆ alkyl, aryl, heteroaryl, C₃₋₈ cycloalkyl and heterocyclyl, or R4a and R4b, together with the heteroatom to which they are joined, can form heterocyclyl,
wherein, when R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R4c, C₁₋₁₀ alkyl, OH, OR4c, OCOR4c, SH, SR4c, SCOR4c, NH₂, NO₂, NHR4c, NHSO₂NH₂, NHSO₂R4c, NR4cCOR4d, NHCOR4c, NHC(NH)NH₂, NR4cR4d, COR4c, CSR4c, CN, COOH, COOR4c, CONH₂, CONHOH, CONHR4c, CONHOR4c, C(NOH)NH₂, CONR4cR4d, SO₂R4c, SO₃H, SO₂NH₂, SO₂NR4cR4d, wherein R4c and R4d are independently selected from C₁₋₆ alkyl, substituted C₁₋₆ alkyl, aryl, heteroaryl, C₃₋₈ cycloalkyl and heterocyclyl, or R4c and R4d, together with the heteroatom to which they are joined, can form heterocyclyl,
wherein, when the substituent of R4 is C₁₋₁₀ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, R4e, C₁₋₁₀ alkyl, OH, OR4e, OCOR4e, SH, SR4e, SCOR4e, NH₂, NO₂, NHR4e, NHSO₂NH₂, NHSO₂R4e, NR4eCOR4f, NHCOR4e, NHC(NH)NH₂, NR4eR4f, COR4e, CSR4e, CN, COOH, COOR4e, CONH₂, CONHOH, CONHR4e, CONHOR4e, C(NOH)NH₂, CONR4eR4f, SO₂R4e, SO₃H, SO₂NH₂, SO₂NR4eR4f, wherein R4e and R4f are independently selected from C₁₋₆ alkyl, substituted C₁₋₆ alkyl, aryl, heteroaryl, C₃₋₈ cycloalkyl and heterocyclyl, or R4e and R4f, together with the heteroatom to which they are joined, can form heterocyclyl;
R5 together with the C to which it is attached, can form a carbonyl group with the double bonds in Formula II rearranged accordingly, or R5 is selected from H, C₁₋₆ alkyl, aryl, heteroaryl, heterocyclyl, C₃₋₈ cycloalkyl, C₁₋₆ alkoxy, aryloxy, heteroaryloxy, heterocyclyloxy, R5a, halogen, OH, OR5a, SH, SR5a, OCOR5a, SCOR5a, NO₂, NHR5a, NHSO₂NH₂, NHSO₂R5a, NR5aCOR5b, NHCOR5a, NHC(NH)NH₂, NR5aR5b, COR5a, CSR5a, CN, COOH, COOR5a, CONH₂, CONHOH, CONHR5a, CONHOR5a, C(NOH)NH₂, CONR5aR5b, SO₂R5a, SO₃H, SO₂NH₂, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl,
wherein, when R5 is C₁₋₆ alkyl, aryl, heteroaryl, heterocyclyl, C₁₋₆ alkoxy, aryloxy, heteroaryloxy, heterocyclyloxy, C₁₋₆ alkyl, C₃₋₈ cycloalkyl, or is a group containing one or more of these moieties, each of these moieties may optionally be substituted with one or more groups selected from halogen, aryl, heteroaryl, heterocyclyl, C₁₋₆ alkoxy, aryloxy, heteroaryloxy, heterocyclyloxy, R5c, C₁₋₆ alkyl, OH, OR5c, OCOR5c, SH, SR5c, SCOR5c, NH₂, NO₂, NHR5c, NHSO₂NH₂, NHSO₂R5c, NR5cCOR5d, NHCOR5c, NHC(NH)NH₂, NR5cR5d, COR5c, CSR5c, CN, COOH, COOR5c, CONH₂, CONHOH, CONHR5c, CONHOR5c, C(NOH)NH₂, CONR5cR5d, SO₂R5c, SO₃H, SO₂NH₂, SO₂NR5cR5d, wherein R5c and R5d are independently selected from C₁₋₆ alkyl, substituted C₁₋₆ alkyl, aryl, heteroaryl, C₃₋₈ cycloalkyl and heterocyclyl, or R5c and R5d, together with the heteroatom to which they are joined, can form heterocyclyl,
wherein, when the substituent of R5 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R5e, C₁₋₆ alkyl, OH, OR5e, OCOR5e, SH, SR5e, SCOR5e, NH₂, NO₂, NHR5e, NHSO₂NH₂, NHSO₂R5e, NR5eCOR5f, NHCOR5e, NHC(NH)NH₂, NR5eR5f, COR5e, CSR5e, CN, COOH, COOR5e, CONH₂, CONHOH, CONHR5e, CONHOR5e, C(NOH)NH₂, CONR5eR5f, SO₂R5e, SO₃H, SO₂NH₂, SO₂NR5eR5f, wherein R5e and R5f are independently selected from C₁₋₆ alkyl, substituted C₁₋₆ alkyl, aryl, heteroaryl, C₃₋₈ cycloalkyl and heterocyclyl, or R5e and R5f, together with the heteroatom to which they are joined, can form heterocyclyl;
X can be O (with the double bonds in Formula II rearranged accordingly), N, CH or C—R6, wherein R6 is selected from C₁₋₆ alkyl, aryl, heteroaryl, heterocyclyl, C₁₋₆ alkoxy, aryloxy, heteroaryloxy, heterocyclyloxy, R6a, halogen, OH, OR6a, SH, SR6a, OCOR6a, SCOR6a, NH₂, NO₂, NHR6a, NHSO₂NH₂, NHSO₂R6a, NR6aCOR6b, NHCOR6a, NHC(NH)NH₂, NR6aR6b, COR6a, CSR6a, CN, COOH, COOR6a, CONH₂, CONHOH, CONHR6a, CONHOR6a, C(NOH)NH₂, CONR6aR6b, SO₂R6a, SO₃H, SO₂NH₂, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl,
wherein, when R6 is heteroaryl or heterocyclyl, each of these moieties may optionally be substituted with one or more oxygen atoms, and when R6 is C₁₋₆ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R6c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR6c, OCOR6c, SH, SR6c, SCOR6c, NH₂, NO₂, NHR6c, NHSO₂NH₂, NHC(NH)NH₂, NHSO₂R6c, NR6cCOR6d, NHCOR6c, NR6cR6d, COR6c, CSR6c, CN, COOH, COOR6c, CONH₂, CONHR6c, CONHOR6c, CONHOH, C(NOH)NH₂, CONR6cR6d, SO₂R6c, SO₃H, SO₂NH₂, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R6c and R6d, together with the heteroatom to which they are joined, can form heterocyclyl,
wherein, when the substituent of R6 is heteroaryl or heterocyclyl, each of these moieties may optionally be substituted with one or more oxygen atoms, or when the substituent of R6 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R6e, C₁₋₆ alkyl, C₁₋₄ alkoxy, OH, OR6e, OCOR6e, SH, SR6e, SCOR6e, NH₂, NO₂, NHR6e, NHSO₂NH₂, NHC(NH)NH₂, NHSO₂R6e, NR6eCOR6f, NHCOR6e, NR6eR6f, COR6e, CSR6e, CN, COOH, COOR6e, CONH₂, CONHOH, CONHR6e, CONHOR6e, C(NOH)NH₂, CONR6eR6f, SO₂R6e, SO₃H, SO₂NH₂, SO₂NR6eR6f, wherein R6e and R6f are independently selected from C₁₋₆ alkyl, substituted C₁₋₆ alkyl, aryl, heteroaryl, C₃₋₈ cycloalkyl and heterocyclyl, or R6e and R6f, together with the heteroatom to which they are joined, can form heterocyclyl;
Y can be N, CH or C—R7, wherein R7 is selected from C₁₋₆ alkyl, aryl, heteroaryl, heterocyclyl, C₁₋₆ alkoxy, aryloxy, heteroaryloxy, heterocyclyloxy, R7a, halogen, OH, OR7a, SH, SR7a, OCOR7a, SCOR7a, NH₂, NO₂, NHR7a, NHSO₂NH₂, NHSO₂R7a, NR7aCOR7b, NHCOR7a, NHC(NH)NH₂, NR7aR7b, COR7a, CSR7a, CN, COOH, COOR7a, CONH₂, CONHOH, CONHR7a, CONHOR7a, C(NOH)NH₂, CONR7aR7b, SO₂R7a, SO₃H, SO₂NH₂, SO₂NR7aR7b, wherein R7a and R7b are independently selected from C₁₋₆ alkyl, substituted C₁₋₆ alkyl, aryl, heteroaryl, C₃₋₈ cycloalkyl and heterocyclyl, or R7a and R7b, together with the heteroatom to which they are joined, can form heterocyclyl,
wherein, when R7 is heteroaryl or heterocyclyl, each of these moieties may optionally be substituted with one or more oxygen atoms, and when R7 is C₁₋₆ alkyl, aryl, heteroaryl, heterocyclyl, C₁₋₆ alkoxy, aryloxy, heteroaryloxy, heterocyclyloxy, C₃₋₈ cycloalkyl or is a group containing one or more of these moieties, each of these moieties may optionally be substituted with one or more groups selected from halogen, R7c, C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, O7c, OCOR7c, SH, SR7c, SCOR7c, NH₂, NO₂, NHR7c, NHSO₂NH₂, NHC(NH)NH₂, NHSO₂R7c, NR7cCOR7d, NHCOR7c, NR7cR7d, COR7c, CSR7c, CN, COOH, COOR7c, CONH₂, CONHR7c, CONHOR7c, CONHOH, C(NOH)NH₂, CONR7cR7d, SO₂R7c, SO₃H, SO₂NH₂, SO₂NR7cR7d, wherein R7c and R7d are independently selected from C₁₋₆ alkyl, substituted C₁₋₆ alkyl, aryl, heteroaryl, C₃₋₈ cycloalkyl and heterocyclyl, or R7c and R7d, together with the heteroatom to which they are joined, can form heterocyclyl,
wherein, when the substituent of R7 is heteroaryl or heterocyclyl, each of these moieties may optionally be substituted with one or more oxygen atoms, or when the substituent of R7 is C₁₋₆ alkyl, C₁₋₆ alkynyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, aryl, heteroaryl, heterocyclyl, aryl C₁₋₆ alkyl, heteroaryl C₁₋₆ alkyl, heterocyclyl C₁₋₆ alkyl, C₁₋₄ alkoxy, R7e, C₁₋₆ alkyl, OH, OR7e, OCOR7e, SH, SR7e, SCOR7e, NH₂, NO₂, NHR7e, NHSO₂NH₂, NHSO₂R7e, NHC(NH)NH₂, NR7eCOR7f, NHCOR7e, NR7eR7f, COR7e, CSR7e, CN, COOH, COOR7e, CONH₂, CONHOH, CONHR7e, CONHOR7e, C(NOH)NH₂, CONR7eR7f, SO₂R7e, SO₃H, SO₂NH₂, SO₂NR7eR7f, wherein R7e and R7f are independently selected from C₁₋₆ alkyl, substituted C₁₋₆ alkyl, aryl, heteroaryl, cycloalkyl and heterocyclyl, or R7e and R7f, together with the heteroatom to which they are joined, can form heterocyclyl;
Z can be N, CH or C—R8, wherein R8 is selected from C₁₋₁₀ alkyl, aryl, heteroaryl, heterocyclyl, C₁₋₆ alkoxy, aryloxy, heteroaryloxy, heterocyclyloxy, R8a, halogen, OH, OR8a, SH, SR8a, OCOR8a, SCOR8a, NH₂, NO₂, NHR8a, NHSO₂NH₂, NHSO₂R8a, NR8aCOR8b, NHCOR8a, NHC(NH)NH₂, NR8aR8b, COR8a, CSR8a, CN, COOH, COOR8a, CONH₂, CONHOH, CONHR8a, CONHOR8a, C(NOH)NH₂, CONR8aR8b, SO₂R8a, SO₃H, SO₂NH₂, SO₂NR8aR8b, wherein R8a and R8b are independently selected from C₁₋₆ alkyl, substituted C₁₋₆ alkyl, aryl, heteroaryl, C₃₋₈ cycloalkyl and heterocyclyl, or R8a and R8b, together with the heteroatom to which they are joined, can form heterocyclyl,
wherein, when R8 is C₁₋₆ alkyl, C₁₋₁₀ alkyl, aryl, heteroaryl, heterocyclyl, C₁₋₆ alkoxy, aryloxy, heteroaryloxy, heterocyclyloxy, C₃₋₈ cycloalkyl, or is a group containing one or more of these moieties, each of these moieties may optionally be substituted with one or more groups selected from halogen, R8c, C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, OH, OR8c, OCOR8c, SH, SR8c, SCOR8c, NH₂, NO₂, NHR5c, NHSO₂NH₂, NHSO₂R8c, NR8cCOR8d, NHCOR5c, NHC(NH)NH₂, NR8cR8d, COR8c, CSR8c, CN, COOH, COOR8c, CONH₂, CONHOH, CONHR8c, CONHOR5c, C(NOH)NH₂, CONR8cR8d, SO₂R8c, SO₃H, SO₂NH₂, SO₂NR8cR8d, wherein R8c and R8d are independently selected from C₁₋₆ alkyl, substituted C₁₋₆ alkyl, aryl, heteroaryl, C₃₋₈ cycloalkyl and heterocyclyl, or R8c and R8d, together with the heteroatom to which they are joined, can form heterocyclyl,
wherein, when the substituent of R8 is C₁₋₆ alkyl, aryl, heteroaryl, heterocyclyl, C₁₋₆ alkoxy, aryloxy, heteroaryloxy, heterocyclyloxy, aryl C₁₋₆ alkyl, heteroaryl C₁₋₆ alkyl, heterocyclyl C₁₋₆ alkyl, aryl C₁₋₆ alkoxy, heteroaryl C₁₋₆ alkoxy, heterocyclyl C₁₋₆ alkoxy, C₃₋₈ cycloalkyl, or is a group containing one or more of these moieties, each of these moieties may optionally be substituted with one or more groups selected from halogen, R8e, C₁₋₆ alkyl, OH, OR8e, OCOR8e, SH, SR8e, SCOR8e, NH₂, NO₂, NHR8e, NHSO₂NH₂, NHSO₂R8e, NR8eCOR8f, NHCOR8e, NHC(NH)NH₂, NR8eR8f, COR8e, CSR8e, CN, COOH, COOR8e, CONH₂, CONHOH, CONHR8e, CONHOR8e, C(NOH)NH₂, CONR8eR8f, SO₂R8e, SO₃H, SO₂NH₂, SO₂NR8eR8f, wherein R8e and R8f are independently selected from C₁₋₆ alkyl, substituted C₁₋₆ alkyl, aryl, heteroaryl, C₃₋₈ cycloalkyl and heterocyclyl, or R8e and R8f, together with the heteroatom to which they are joined, can form heterocyclyl;
wherein, at most, two of the atoms or groups denoted X, Y and Z can be N;
wherein, when W is N, the CONR1R2 group may be joined to W instead, with the double bonds in Formula I rearranged accordingly.

2. The process according to claim 1, wherein the compound of Formula II or Formula I has a formula selected from Formula IIa, Formula IIb, Formula IIc, Formula IId and Formula Ia 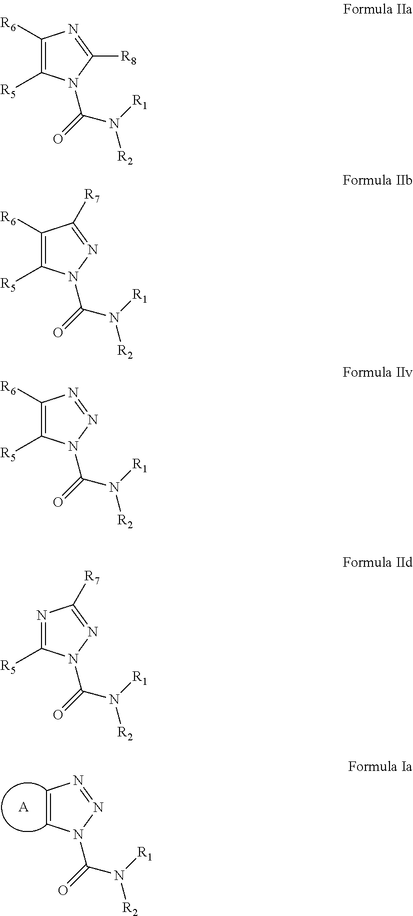
and wherein the intermediate of Formula II′ or Formula I′ has a corresponding structure in which the —CONR1R2 group of Formula IIa-d or Formula Ia is replaced by H.

3. A process according to claim 1 or claim 2, wherein the compound has the Formula IIa, and wherein the intermediate of Formula II′ has a corresponding structure in which the —CONR1R2 group of Formula IIa is replaced by H.

4. The process according to claim 3, wherein the compound is of Formula IIa, and wherein:
R1 is selected from H and C₁₋₄ alkyl,
R2 is selected from C₁₋₆ alkyl, aryl, heteroaryl, heterocyclyl, C₃₋₁₀ cycloalkyl, aryl C₁₋₆ alkyl, heteroaryl C₁₋₆ alkyl, heterocyclyl C₁₋₆ alkyl and C₃₋₁₀ cycloalkyl C₁₋₆ alkyl, each of which may optionally be substituted with one or more groups selected from R2a, halogen, OH, OR2a, OCOR2a, SH, SR2a, SCOR2a, NH₂, NHR2a, NHSO₂NH₂, NHSO₂R2a, NR2aCOR2b, NHC(NH)NH₂, NHCOR2a, NR2aR2b, COR2a, CSR2a, CN, COOH, COOR2a, CONH₂, CONHOH, CONHR2a, CONHOR2a, C(NOH)NH₂, SO₂R2a, SO₃H, SO₂NH₂, CONR2aR2b, SO₂NR2aR2b, wherein R2a and R2b are independently selected from C₁₋₆ alkyl, substituted C₁₋₆ alkyl, aryl, heteroaryl, C₃₋₈ cycloalkyl and heterocyclyl, or R2a and R2b, together with the heteroatom to which they are joined, can form heterocyclyl,
wherein, when the substituent of R2 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R2c, halogen, OH, OR2c, OCOR2c, SH, SR2c, SCOR2c, NH₂, NHR2c, NHSO₂NH₂, NHSO₂R2c, NR2cCOR2d, NHC(NH)NH₂, NHCOR2c, NR2cR2d, COR2c, CSR2c, CN, COOH, COOR2c, CONH₂, CONHOH, CONHR2c, CONHOR2c, C(NOH)NH₂, SO₂R2c, SO₃H, SO₂NH₂, CONR2cR2d, SO₂NR2cR2d, wherein R2c and R2d are independently selected from C₁₋₆ alkyl, substituted C₁₋₆ alkyl, aryl, heteroaryl, C₃₋₈ cycloalkyl and heterocyclyl, or R2c and R2d, together with the heteroatom to which they are joined, can form heterocyclyl,
R5 is selected from H, R5a, halogen, OH, OR5a, OCOR5a, SH, SR5a, SCOR5a, NH₂, NHR5a, NHSO₂NH₂, NHSO₂R5a, NR5aCOR5b, NHC(NH)NH₂, NHCOR5a, NR5aR5b, COR5a, CSR5a, CN, COOH, COOR5a, CONH₂, CONHOH, CONHR5a, CONHOR5a, C(NOH)NH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, aryl, heteroaryl, C₃₋₅ cycloalkyl and heterocyclyl, or R5a and R5b, together with the heteroatom to which they are joined, can form heterocyclyl,
R6 is selected from aryl, heteroaryl, heterocyclyl, C₃₋₁₀ cycloalkyl, each of which may optionally be substituted with one or more groups selected from R6a, halogen, OH, OR6a, OCOR6a, SH, SR6a, SCOR6a, NO₂, NH₂, NHR6a, NHSO₂NH₂, NHSO₂R6a, NR6aCOR6b, NHC(NH)NH₂, NHCOR6a, NR6aR6b, COR6a, CSR6a, CN, COOH, COOR6a, CONH₂, CONHOH, CONHR6a, CONHOR6a, C(NOH)NH₂, SO₂R6a, SO₃H, SO₂NH₂, CONR6aR6b, SO₂NR6aR6b, wherein R6a and R6b are independently selected from C₁₋₆ alkyl, substituted C₁₋₆ alkyl, aryl, heteroaryl, C₃₋₈ cycloalkyl and heterocyclyl, or R6a and R6b, together with the heteroatom to which they are joined, can form heterocyclyl, and wherein, when R6 is heteroaryl or heterocyclyl, each of these moieties may optionally be substituted with one or more oxygen atoms,
wherein, when the substituent of R6 is C₁₋₆ alkyl, substituted C₁₋₆ alkyl, aryl, heteroaryl, C₃₋₈ cycloalkyl, heterocyclyl or a group containing one or more of these moieties, each of these moieties may optionally be substituted with one or more groups selected from R6c, halogen, OH, OR6c, OCOR6c, SH, SR6c, SCOR6c, NH₂, NHR6c, NHSO₂NH₂, NHSO₂R6c, NR6cCOR6d, NHC(NH)NH₂, NHCOR6c, NR6cR6d, COR6c, CSR6c, CN, COOH, COOR6c, CONH₂, CONHOH, CONHR6c, CONHOR6c, C(NOH)NH₂, SO₂R6c, SO₃H, SO₂NH₂, CONR6cR6d, SO₂NR6cR6d, wherein R6c and R6d are independently selected from C₁₋₆ alkyl, substituted C₁₋₆ alkyl, aryl, heteroaryl, C₃₋₈ cycloalkyl and heterocyclyl, or R6c and R6d, together with the heteroatom to which they are joined, can form heterocyclyl, and wherein, when the substituent of R6 is heteroaryl or heterocyclyl, each of these moieties may optionally be substituted with one or more oxygen atoms, and
R8 is selected from H, R5a, halogen, OH, OR5a, OCOR5a, SH, SR5a, SCOR5a, NH₂, NHR5a, NHSO₂NH₂, NHSO₂R5a, NR5aCOR5b, NHC(NH)NH₂, NHCOR5a, NR5aR5b, COR5a, CSR5a, CN, COOH, COOR5a, CONH₂, CONHOH, CONHR5a, CONHOR5a, C(NOH)NH₂, SO₂R5a, SO₃H, SO₂NH₂, CONR5aR5b, SO₂NR5aR5b, wherein R5a and R5b are independently selected from C₁₋₆ alkyl, substituted C₁₋₆ alkyl, aryl, heteroaryl, C₃₋₈ cycloalkyl and heterocyclyl, or R5a and R5b, together with the heteroatom to which they are joined, can form heterocyclyl.

5. The process according to claim 4, wherein R1 is selected from H, methyl and ethyl, and R2 is selected from aryl, heteroaryl, heterocyclyl, and C₃₋₁₀ cycloalkyl each of which may be substituted or unsubstituted.

6. The process according to claim 4 or 5, wherein R2 is selected from fully saturated heterocyclyl and C₅₋₈ cycloalkyl, each of which are monocyclic and may be substituted or unsubstituted.

7. The process according to claim 6, wherein R2 is an unsubstituted cyclopentyl or unsubstituted cyclohexyl.

8. The process according to claim 6, wherein R2 is a fully saturated heterocyclyl, and wherein the heterocyclyl ring contains a single heteroatom, such as nitrogen or oxygen.

9. The process according to claim 8, wherein the heterocyclyl R2 is six membered and the heteroatom in the said heterocyclyl group is at the 4-position relative to the position of attachment of the heterocyclyl group R2 to the urea nitrogen.

10. The process according to claim 9, wherein the heteroatom in heterocyclyl R2 is a nitrogen heteroatom which is substituted with a group selected from CN, CONH₂, C(NOH)NH₂, SO₂—C₁₋₄ alkyl, SO₂-aryl, CO-heteroaryl, CO—C₁₋₄ alkyl, COO—C₁₋₄ alkyl, COO-aryl, C₁₋₄ alkyl, aryl C₁₋₃ alkyl, heteroaryl C₁₋₃ alkyl, heterocyclyl C₁₋₃ alkyl, aryl, heteroaryl, and heterocyclyl, wherein the C₁₋₄ alkyl may optionally be substituted with OH, CN, COOH, the SO₂-aryl may optionally be substituted with a C₁₋₄ alkyl or C₁₋₄ haloalkyl, the CO-heteroaryl may optionally be substituted with a heteroaryl or halogen, the heteroaryl C₁₋₃ alkyl may optionally be substituted with COO—C₁₋₃ alkyl, and the heteroaryl may optionally be substituted with one or more halogens.

11. The process according to claim 10, wherein the nitrogen heteroatom is substituted with phenyl C₁₋₃ alkyl.

12. The process according to any one of claims 4 to 11, wherein R6 is selected from monocyclic aryl, monocyclic heteroaryl, and heterocyclyl, each of which may be substituted or unsubstituted.

13. The process according to claim 12, wherein R6 is a substituted aryl, and wherein said aryl is substituted with one or more groups selected from halogen, R6a, OH, OR6a, NH₂, NO₂, NHC(NH)NH₂, NHR6a, NR6aR6b, C(NOH)NH₂, COR6a, COOH, COOR6a, CONH₂, CONHOH, SO₂R6a, SO₂NR6aR6b, wherein R6a and R6b are independently selected from C₁₋₆ alkyl, substituted C₁₋₆ alkyl, aryl, heteroaryl, C₃₋₈cycloalkyl and heterocyclyl,
wherein, when the substituent of R6 is C₁₋₆ alkyl, substituted C₁₋₆ alkyl, aryl, heteroaryl, C₃₋₈ cycloalkyl, heterocyclyl or is a group containing one or more of these moieties, each of these moieties: may optionally be substituted with one or more groups selected from OR6c, OH, and CONH₂, wherein R6c is selected from C₁₋₆ alkyl, substituted C₁₋₆ alkyl, aryl, heteroaryl, C₃₋₈ cycloalkyl and heterocyclyl, and wherein, when the substituent of R6 is heteroaryl or heterocyclyl, each of these moieties may optionally be substituted with one or more oxygen atoms.

14. The process according to claim 13, wherein R6 is a substituted aryl which is substituted with one or more groups selected from halogen, OH, NO₂, C₁₋₄ alkoxy, CONH₂, C(NOH)NH₂, CONHOH, SO₂—C₁₋₄ alkyl, heterocyclyl, and aryl, wherein the heterocyclyl may optionally be substituted with an oxygen atom and the aryl may optionally be substituted with CONH₂.

15. The process according to claim 12, wherein R6 is a heterocyclyl which is optionally substituted with an oxygen atom.

16. The process according to claim 12, wherein R6 is a monocyclic heteroaryl which is optionally substituted with an oxygen atom.

17. The process according to any of claims 3 to 16, wherein R8 is H.

18. The process according to any of claims 3 to 17, wherein R5 is H.

19. The process according to any preceding claim, wherein Hal in the carbamoyl halide having the formula R1R2NC(═O)Hal represents Cl.

20. A process according to any of claims 1 to 3, wherein in the carbamoyl halide having the formula R1R2NC(═O)Hal, both of R1 and R2 are other than H.

21. A process according to any of claims 1 to 3, wherein R1 is C₁₋₂₀ alkyl.

22. A process according to any of claims 1 to 3, wherein R2 is C₃₋₁₀ cycloalkyl.

23. A process according to any of claims 1 to 3, wherein R6 is heteroaryl.

24. A process according to claim 23, wherein R6 is pyridyl.

25. A process according to claim 24, wherein the urea of Formula IIa is subjected to a further step of N-oxidation of the pyridine R6.

26. A process according to claim 25, wherein the N-oxidation is conducted using a peroxyacid, such as peracetic acid.

27. A process according to claim 25 or claim 26, for the preparation of 3-(1-(cyclohexyl(methyl)carbamoyl-1H-imidazol-4-yl)pyridine 1-oxide.

28. A process according to any preceding claim, wherein the carbamoyl halide is a carbamoyl chloride, prepared by subjecting an amine R1R2NH to carbamoylation using a phosgene reagent, such as triphosgene.

29. A process according to claim 28, wherein the carbamoylation is conducted in dichloromethane, in the presence of a base.

30. A process according to claim 28 or claim 29, wherein the carbamoyl chloride is not isolated before addition of the intermediate of Formula II′ or Formula I′.

31. A process according to any preceding claim, wherein the intermediate of Formula II′ has a structure according to Formula i: 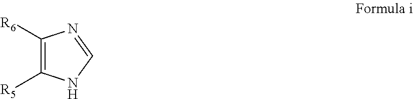
wherein R5 and R6 are as defined according to any preceding claim.

32. A process according to claim 31, wherein the intermediate of Formula i is prepared from a mercaptoimidazole having the structure: 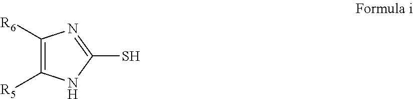
wherein R5 and R6 are as defined in claim 31, or an imidazolethione tautomer thereof, using Raney nickel or a sodium nitrite/nitric acid mixture.

33. A process according to claim 32, wherein the mercaptoimidazole or imidazolethione tautomer thereof has R5 as H, and is prepared by treatment of an aminoketone of Formula ii: 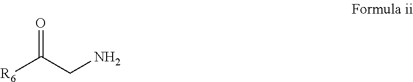
wherein R6 is as defined is claim 32, or a salt thereof, with thiocyanate.

34. A process according to claim 31, wherein the intermediate of Formula i, wherein R5 is H, is prepared by formylation of an aminoketone of Formula ii: 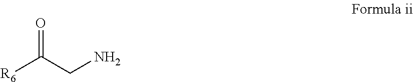
wherein R6 is as defined in claim 31, or a salt thereof, followed by reaction of the —NHCHO derivative so formed with an ammonium salt.

35. A process according to claim 33, wherein the aminoketone or salt of Formula ii is prepared by acid hydrolysis of an azirine derivative of formula iii 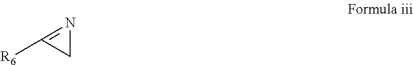
wherein R6 is as defined in claim 33.

36. A process according to claim 35, wherein the azirine derivative of formula iii is prepared by subjecting a ketoxime tosylate derivative of formula iv: 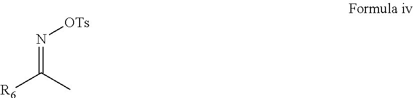
wherein R6 is as defined in claim 35 and OTs represents toluenesulphonate, to treatment with a base.

37. A process according to claim 36, wherein the ketoxime tosylate of Formula iv is prepared from the corresponding ketoxime: R6C(═N—OH)CH₃, wherein R6 is as defined in claim 36, by reaction with tosyl chloride.

38. A process according to claim 37, wherein the ketoxime R6C(═N—OH)CH₃ is prepared from the corresponding acetyl derivative of R6: R6-C(═O)CH₃, wherein R6 is as defined in claim 37, by reaction thereof with hydroxylamine.

39. A process for the preparation of an aminoketone of Formula ii: 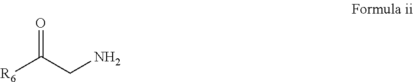
or a salt thereof, wherein R6 is as defined according to any preceding claim, the process comprising the tosylation of the corresponding ketoxime: R6C(═N—OH)CH₃, using tosyl chloride in the presence of a first base and in a solvent comprising a C₁₋₆ alcohol, followed by treatment of the resulting ketoxime tosylate, without isolation, with a second base in a solvent comprising a C₁₋₆ alcohol to yield the corresponding azirine derivative of Formula iii: 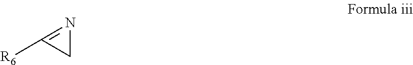
followed by acid hydrolysis of the azirine derivative to yield the aminoketone or salt of Formula ii.

40. A process according to claim 39, wherein the first base, employed during the tosylation step, is a butoxide salt, such as sodium t-butoxide.

41. A process according to claim 39 or 40, wherein the solvent used in the tosylation step comprises butanol, such as t-butanol, optionally together with methyl-t-butyl ether.

42. A process according to any of claims 39 to 41, wherein the base and alcoholic solvent are added to the ketoxime, followed by addition of the tosyl chloride in portions.

43. A process according to any of claims 39 to 42, wherein the second base, employed during the production of the azirine derivative, is a methoxide salt, such as sodium methoxide.

44. A process according to any of claims 39 to 43, wherein the solvent used during the production of the azirine derivative is methanol.

45. A process according to any of claims 39 to 44, wherein the resulting aminoketone of Formula ii is used to prepare an intermediate of Formula i according to claim 31, by means of the steps of claims 32 and 33, or claim 34.

46. A process for preparing an aminoketone of Formula ii: 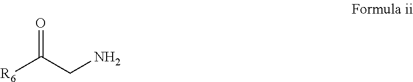
or a salt thereof, wherein R6 is as defined according to any preceding claim, the process comprising the reaction of the corresponding acetyl derivative of R6: R6-C(═O)CH₃, with hydroxylamine in a solvent consisting essentially of pyridine, followed by tosylation of the resulting ketoxime, without isolation thereof, using tosyl chloride, followed by treatment of the resulting ketoxime tosylate with a base in a solvent comprising a C₁₋₆ alcohol, to produce the corresponding azirine derivative of Formula iii: 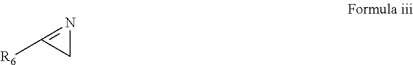
followed by acid hydrolysis of the azirine derivative to yield the aminoketone or salt of Formula ii.

47. A process according to claim 46, wherein the base used in the conversion of the ketoxime tosylate to the azirine is DBU.

48. A process according to claim 36 or claim 39, wherein the base used in the conversion of the ketoxime tosylate to the azirine is DBU.

49. A process for preparing an azirine derivative of Formula iii 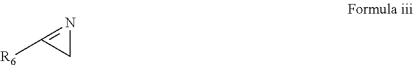
wherein R6 is as defined according to any preceding claim, the process comprising subjecting a ketoxime tosylate of Formula iv: 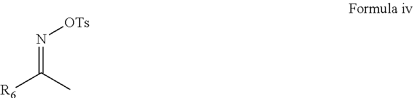
to treatment with a base, wherein the base comprises DBU.

50. A process for preparing an aminoketone of Formula ii 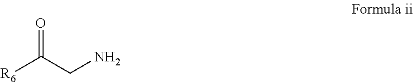
wherein an azirine derivative of Formula iii prepared according to claim 49 is subjected to acid hydrolysis.

51. A substituted urea compound of Formula II or Formula I as defined in claim 1, obtained or obtainable by the process of any of claims 1 to 38, or by a process in which a process according to any of claims 39 to 50 is comprised.
